(19) United States
(12) Patent Application Publication
(10) Pub. No.: US 2025/0153157 A1
(43) Pub. Date: May 15, 2025

(54) PALLADIUM PRECATALYST EMBODIMENTS FOR ENANTIOSELECTIVE CHEMICAL REACTIONS AND METHODS OF MAKING AND USING THE SAME

(71) Applicants: UVic Industry Partnerships Inc., Victoria (CA); Queen Mary University of London, London (GB)

(72) Inventors: David Leitch, Victoria (CA); Stellios Arseniyadis, London (GB); Jingjun Huang, Victoria (CA)

(21) Appl. No.: 18/838,903

(22) PCT Filed: Feb. 17, 2023
(86) PCT No.: PCT/IB2023/051474

§ 371 Date: Aug. 15, 2024

Related U.S. Application Data
(60) Provisional application No. 63/311,812, filed on Feb. 18, 2022.

(51) Int. Cl. B01J 31/22; B01J 31/18; B01J 31/24; B01J 35/77; B01J 37/04; C07C 67/31; C07C 67/347

(52) U.S. Cl. CPC B01J 31/2295; B01J 31/189; B01J 31/2414; B01J 31/2447; B01J 35/77; B01J 37/04; C07C 67/31; C07C 67/347; B01J 2231/44; B01J 2231/485; B01J 2531/004; B01J 2531/0288; B01J 2531/824; C07C 2601/16

(57) ABSTRACT

Disclosed herein are embodiments of a chiral Pd(0) precatalyst that exhibits bench-top and/or solution stability against degradation and/or oxidation. Also disclosed are method embodiments for making the Pd(0) precatalyst and methods for using the same in enantioselective chemical reactions, such as carbon-element bond formation.

### CROSS REFERENCE TO RELATED APPLICATIONS

This application claims the benefit of and priority to the earlier filing date of U.S. Provisional Patent Application No. 63/311,812, filed on Feb. 18, 2022; this prior application is incorporated herein by reference in its entirety.

### FIELD

The present disclosure concerns palladium precatalyst embodiments for use in enantioselective carbon-element bond formation and methods of making and using the same.

### BACKGROUND

Most cross-coupling requires the aid of a transition metal catalyst to proceed. Pd(0)-containing catalysts are the most widely used and studied. Early methods of generating Pd(0) catalysts employed Pd(II) salts, such as palladium dichloride and palladium acetate, as precatalysts; however, the reduction pathway involved with such catalysts can be unreliable and is often very condition-dependent. Another method is to directly use a Pd(0)Lx complex as a precatalyst, such as Pd(PPh₃)₄ or bis(dibenzylideneacetone)palladium(0) (or “Pd(dba)₂”); however, these compounds also come with drawbacks. For example, Pd(0)-dba complexes can produce inconsistent catalytic results depending on source, degrade rapidly in solution to produce palladium nanoparticles (palladium black), and the dba released after activation is “non-innocent” and can interfere with catalysis. Pd(II)-containing precatalysts exist; however, such precatalysts still require basic conditions to be activated, and the pre-installation of the phosphine ligand makes each precatalyst specific to the substrates being coupled. And, not every phosphine that could be used in cross-coupling is available as part of a precatalyst. There exists a need in the art for new chiral Pd(0) precatalysts that are stable and that have ligands already installed so as to allow the precatalysts to directly enter the cross-coupling catalytic cycle.

### SUMMARY

Disclosed herein are embodiments of a chiral precatalyst having a structure according to Formulas described herein. Also disclosed are method embodiments for use in making the chiral precatalyst, wherein the method comprises exposing a Pd(0) precursor complex having a structure according to Formula A to a donor atom-containing ligand compound having a structure according to Formula V, wherein Formulas A and V are described herein. Also disclosed herein are embodiments of a method, comprising using the chiral precatalyst according to the present disclosure as a catalyst in a palladium-mediated enantioselective chemical reaction, such as an asymmetric allylation reaction.

The foregoing and other objects and features of the present disclosure will become more apparent from the following detailed description, which proceeds with reference to the accompanying figures.

### BRIEF DESCRIPTION OF THE DRAWINGS

FIG. 1 is an image showing the solid-state molecular structure of a Pd(0) precatalyst embodiment (precatalyst 2), including the THF solvate.

FIG. 2 is an image showing the solid-state molecular structure of a Pd(0) precatalyst embodiment (precatalyst 4), including the THF solvate.

FIG. 3 is an image showing the solid-state molecular structure of oxidized precatalyst 4, including TBME solvate.

FIG. 4 is an image showing the solid-state molecular structure of a Pd(0) precatalyst embodiment (precatalyst 6).

FIG. 5 shows combined ³¹P nuclear magnetic resonance spectra of Pd(0) precatalyst 1 according to the present disclosure in d₂-DCM and d₈-THF, showing two conformers of the complex (1-exo and 1-endo).

FIG. 6 is a graph of mole fraction of the 1-endo conformer of Pd(0) precatalyst 1 as a function of volume fraction of THF.

FIG. 7 shows calculated structures of the 1-endo and 1-exo conformers of Pd(0) precatalyst 1 with relative free energies in the gas phase, DCM, and THF (implicit solvation models).

FIG. 8 is a graph of concentration versus time showing stability of Pd(0) precatalysts 1-6 in THF under N₂, showing no decomposition over at least 48 hours, as well as Pd(0) precatalyst 1 and L₁+Pd₂dba₃·CHCl₃ under air, wherein slow decomposition of Pd(0) precatalyst 1 (>80% intact after 48 hours) can be seen compared to rapid decomposition of [L₁]Pd(dba) (<50% remaining after 30 minutes).

FIG. 9 is a plot of normalized Pd(0) precatalyst concentration for six different Pd(0) precatalysts over 48 hours at room temperature.

FIG. 10 is a plot of normalized Pd(0) precatalyst 1 concentration and an oxidation by-product in THF over 48 hours at room temperature in air.

FIG. 11 is a plot of normalized ^{Ph}DACH-Pd-dba concentration and a Pd(II) oxidation by-product in THF over 48 hours at room temperature in air and N₂.

FIG. 12 is a plot of mole fraction for the minor conformer of Pd(0) precatalyst 1 in different THF/DCM ratios, wherein the mole fraction is indicated by the ratio between its integration and the total integration of both major and minor conformers.

FIG. 13 is a bar graph showing the conformer changes of Pd(0) precatalyst 1 in DCM mixed with five different solvents, separately.

FIG. 14 is a schematic summary showing a screening process to establish ligand selection and reagents (e.g., solvents and bases) for carrying out an enantioselective reaction using Pd(0) precatalyst embodiments described herein.

### DETAILED DESCRIPTION

### I. Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently. Additionally, the description sometimes uses terms like “produce” and “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and complexes and/or catalysts similar or equivalent to those described herein can be used in the practice or testing of the present disclosure, suitable methods and complexes and/or catalysts are described below. The complexes and/or catalysts, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

Compound embodiments disclosed herein may contain one or more asymmetric elements such as stereogenic centers, stereogenic axes and the like, e.g., asymmetric carbon atoms, so that the chemical conjugates can exist in different stereoisomeric forms. These compound embodiments can be, for example, racemates or optically active forms. For compound with two or more asymmetric elements, these compound can additionally be mixtures of diastereomers. For compound having asymmetric centers, all optical isomers in pure form and mixtures thereof are encompassed by corresponding generic formulas unless context clearly indicates otherwise or an express statement excluding an isomer is provided. In these situations, the single enantiomers, i.e., optically active forms, can be obtained by method known to a person of ordinary skill in the art, such as asymmetric synthesis, synthesis from optically pure precursors, or by resolution of the racemates. Resolution of the racemates can also be accomplished, for example, by conventional methods, such as crystallization in the presence of a resolving agent, or chromatography, using, for example a chiral HPLC column. All isomeric forms are contemplated herein regardless of the methods used to obtain them.

To facilitate review of the various embodiments of the disclosure, the following definitions and explanations of specific terms are provided. Certain functional group terms include a symbol “—” which is used to show how the defined functional group attaches to, or within, the disclosed compound to which it is bound. Also, a dashed bond (i.e., “ [symbol] ”), as used in certain formulas described herein, indicates an optional bond (that is, a bond that may or may not be present). In certain formulas described herein, an asterisk (*) indicates either a chiral substituent or the presence of an asymmetric carbon atom. A person of ordinary skill in the art would recognize that the definitions provided below and compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated on a carbon atom. For example, a phenyl ring that is drawn as

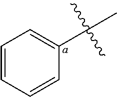 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

Alicyclic: A cyclic hydrocarbon group that can comprise one or more ring systems, including spirocyclic, bicyclic, and/or fused cyclic ring systems.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well.

Aliphatic-aromatic: An aromatic group that is or can be coupled to a compound disclosed herein, wherein the aromatic group is or becomes coupled through an aliphatic group.

Aliphatic-aryl: An aryl group that is or can be coupled to a compound disclosed herein, wherein the aryl group is or becomes coupled through an aliphatic group.

Aliphatic-heteroaryl: A heteroaryl group that is or can be coupled to a compound disclosed herein, wherein the heteroaryl group is or becomes coupled through an aliphatic group.

Alkenyl: An unsaturated monovalent hydrocarbon having at least two carbon atoms to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkoxy: —O-aliphatic, such as —O-alkyl, —O-alkenyl, —O-alkynyl; with exemplary embodiments including, but not limited to, methoxy, ethoxy, n-propoxy, isopropoxy, n-butoxy, t-butoxy, sec-butoxy, n-pentoxy (wherein any of the aliphatic components of such groups can comprise no double or triple bonds, or can comprise one or more double and/or triple bonds).

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atoms to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Amino: —NR^{a}R^{b}, wherein each of R^{a} and R^{b} independently is selected from hydrogen, aliphatic, heteroaliphatic, haloaliphatic, haloheteroaliphatic, aromatic, or an organic functional group.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the HOckel rule (4n+2). The point of attachment to the parent structure typically is through an aromatic portion of the condensed ring system. For example,

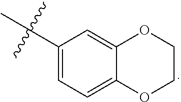 [SMILES: *c1ccc2c(c1)OCCO2]

However, in certain examples, context or express disclosure may indicate that the point of attachment is through a non-aromatic portion of a condensed ring system that is formed by an organic functional group comprising a combination of an aromatic ring and an aliphatic group. For example,

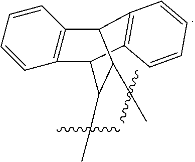 [SMILES: *C1C2c3ccccc3C(c3ccccc32)C1*]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the complexes and/or catalysts disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Catalyst: A substance, usually present in small amounts relative to reactants, which increases the rate of a chemical reaction without itself being consumed or undergoing a chemical change. A catalyst also may enable a reaction to proceed under different conditions (e.g., at a lower temperature) than otherwise possible.

Electron-Withdrawing Group: A functional group capable of accepting electron density from an aromatic ring or olefin moiety to which it is directly attached, such as by inductive electron withdrawal. Representative and non-limiting examples of electron-withdrawing groups can include certain groups, such as aldehyde, ketone, ester, carboxylic acid, acyl, a quaternary amine, acyl halide, cyano, sulfonate, nitro, nitroso, pyridinyl, pyrimidinyl, alkyl halide, halogen (e.g., chloro, bromo, fluoro, or iodo), haloaliphatic, ammonium, or amide.

Ether: -aliphatic-O-aliphatic, -aliphatic-O-aromatic, -aromatic-O-aliphatic, or -aromatic-O-aromatic.

Halo (or halide or halogen): Fluoro, chloro, bromo, or iodo.

Haloaliphatic: An aliphatic group wherein one or more hydrogen atoms, such as one to 10 hydrogen atoms, independently is replaced with a halogen atom, such as fluoro, bromo, chloro, or iodo.

Haloaliphatic-aryl: An aryl group that is or can be coupled to a compound disclosed herein, wherein the aryl group is or becomes coupled through a haloaliphatic group.

Haloaliphatic-heteroaryl: A heteroaryl group that is or can be coupled to a compound disclosed herein, wherein the heteroaryl group is or becomes coupled through a haloaliphatic group.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic. In some embodiments, a fluorophore can also be described herein as a heteroaliphatic group, such as when the heteroaliphatic group is a heterocyclic group.

Heteroaliphatic-aryl: An aryl group that is or can be coupled to a compound disclosed herein, wherein the aryl group is or becomes coupled through a heteroaliphatic group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group. In some embodiments, a fluorophore can also be described herein as a heteroaryl group.

Heteroatom: An atom other than carbon or hydrogen, such as (but not limited to) oxygen, nitrogen, sulfur, silicon, boron, selenium, or phosphorous. In particular disclosed embodiments, such as when valency constraints do not permit, a heteroatom does not include a halogen atom.

Heterocyclic: A heterocyclic group comprising one or more heteroatoms and that can comprise one or more ring systems, including spirocyclic, bicyclic, and/or fused cyclic ring systems.

Organic Functional Group: A functional group that may be provided by any combination of aliphatic, heteroaliphatic, aromatic, and/or haloaliphatic groups, or that may be selected from, but not limited to, aldehyde (i.e., —C(O)H); aroxy (i.e., —O-aromatic); acyl halide (i.e., —C(O)X, wherein X is a halogen, such as Br, F, I, or Cl); halogen; nitro (i.e., —NO₂); cyano (i.e., —CN); azide (i.e., —N₃); carboxyl (i.e., —C(O)OH); carboxylate (i.e., —C(O)O⁻ or salts thereof, wherein the negative charge of the carboxylate group may be balanced with an M⁺ counterion, wherein M⁺ may be an alkali ion, such as κ⁺, Na⁺, Li⁺; an ammonium ion, such as ⁺N(R^{b})₄ where R^{b} is H, aliphatic, heteroaliphatic, haloaliphatic, or aromatic; or an alkaline earth ion, such as [Ca²⁺]_{0.5}, [Mg²⁺]_{0.5}, or [Ba²⁺]_{0.5}); amide (i.e., —C(O)NR^{a}R^{b} or —NR^{a}C(O)R^{b} wherein each of R^{a} and R^{b} independently is selected from hydrogen, aliphatic, heteroaliphatic, haloaliphatic, aromatic, or an organic functional group); ketone (i.e., —C(O)R^{a}, wherein R^{a} is selected from aliphatic, heteroaliphatic, haloaliphatic, aromatic, or an organic functional group); carbonate (i.e., —OC(O)OR^{a}, wherein R^{a} is selected from aliphatic, heteroaliphatic, haloaliphatic, aromatic, or an organic functional group); imine (i.e., —C(═NR^{a})R^{b} or —N═CR^{a}R^{b}, wherein R^{a} and R^{b} independently is selected from hydrogen, aliphatic, heteroaliphatic, haloaliphatic, aromatic, or an organic functional group); azo (i.e., —N═NR^{a} wherein R^{a} is hydrogen, aliphatic, heteroaliphatic, haloaliphatic, aromatic, or an organic functional group); carbamate (i.e., —OC(O)NR^{a}R^{b}, wherein each of R^{a} and R^{b} independently is selected from hydrogen, aliphatic, heteroaliphatic, haloaliphatic, aromatic, or an organic functional group); hydroxyl (i.e., —OH); thiol (i.e., —SH); sulfonyl (i.e., —SO₂R^{a}, wherein R^{a} is selected from hydrogen, aliphatic, heteroaliphatic, haloaliphatic, aromatic, or an organic functional group); sulfonate (i.e., —SO₃⁻, wherein the negative charge of the sulfonate group may be balanced with an M⁺ counter ion, wherein M⁺ may be an alkali ion, such as κ⁺, Na⁺, Li⁺; an ammonium ion, such as ⁺N(R^{b})₄ where R^{b} is H, aliphatic, heteroaliphatic, haloaliphatic, or aromatic; or an alkaline earth ion, such as [Ca²⁺]_{0.5}, [Mg²⁺]_{0.5}, or [Ba²⁺]_{0.5}); oxime (i.e., —CR^{a}═NOH, wherein R^{a} is hydrogen, aliphatic, heteroaliphatic, haloaliphatic, aromatic, or an organic functional group); sulfonamide (i.e., —SO₂NR^{a}R^{b} or —N(R^{a})SO₂R^{b}, wherein each of R^{a} and R^{b} independently is selected from hydrogen, aliphatic, heteroaliphatic, haloaliphatic, aromatic, or an organic functional group); ester (i.e., —C(O)OR^{a} or —OC(O)R^{a}, wherein R^{a} is selected from aliphatic, heteroaliphatic, haloaliphatic, aromatic, or an organic functional group); thiocyanate (i.e., —S—CN or —N═C═S); thioketone (i.e., —C(S)R^{a} wherein R^{a} is selected from hydrogen, aliphatic, heteroaliphatic, haloaliphatic, aromatic, or an organic functional group); thiocarboxylic acid (i.e., —C(O)SH, or —C(S)OH); thioester (i.e., —C(O)SR^{a} or —C(S)OR^{a} wherein R^{a} is selected from hydrogen, aliphatic, heteroaliphatic, haloaliphatic, aromatic, or an organic functional group); dithiocarboxylic acid or ester (i.e., —C(S)SR^{a} wherein R^{a} is selected from hydrogen, aliphatic, heteroaliphatic, haloaliphatic, aromatic, or an organic functional group); phosphonate (i.e., —P(O)(OR^{a})₂, wherein each R^{a} independently is hydrogen, aliphatic, heteroaliphatic, haloaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as κ⁺, Na⁺, Li⁺; an ammonium ion, such as ⁺N(R^{b})₄ where R^{b} is H, aliphatic, heteroaliphatic, haloaliphatic, or aromatic; or an alkaline earth ion, such as [Ca²⁺]_{0.5}, [Mg²⁺]_{0.5}, or [Ba²⁺]_{0.5}); phosphate (i.e., —O—P(O)(OR^{a})₂, wherein each R^{a} independently is hydrogen, aliphatic, heteroaliphatic, haloaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as κ⁺, Na⁺, Li⁺; an ammonium ion, such as ⁺N(R^{b})₄ where R^{b} is H, aliphatic, heteroaliphatic, haloaliphatic, or aromatic; or an alkaline earth ion, such as [Ca²⁺]_{0.5}, [Mg²⁺]_{0.5}, or [Ba²⁺]_{0.5}); silyl ether (i.e., —OSiR^{a}R^{b}, wherein each of R^{a} and R^{b} independently is selected from hydrogen, aliphatic, heteroaliphatic, haloaliphatic, aromatic, or an organic functional group); sulfinyl (i.e., —S(O)R^{a}, wherein R^{a} is selected from hydrogen, aliphatic, heteroaliphatic, haloaliphatic, aromatic, or an organic functional group); thial (i.e., —C(S)H); or combinations thereof.

Peroxy: —O—OR^{a} wherein R^{a} is hydrogen, aliphatic, heteroaliphatic, haloaliphatic, haloheteroaliphatic, aromatic, or an organic functional group.

Precatalyst: A chiral chemical complex comprising a donor atom-based ligand that is coordinated and/or bound to a palladium atom through one or more donor atoms, wherein the palladium atom has an oxidation state of zero (0) and is further coordinated to a compound comprising at least one olefin moiety.

Thioether: —S-aliphatic or —S-aromatic, such as —S-alkyl, —S-alkenyl, —S-alkynyl, —S-aryl, or —S— heteroaryl; or -aliphatic-S-aliphatic, -aliphatic-S-aromatic, -aromatic-S-aliphatic, or -aromatic-S-aromatic.

### II. Introduction

Homogeneous catalysis by transition metal complexes is one of the most powerful technologies in synthetic chemistry. Many of the most efficient and selective methods for the construction of organic molecules and materials are based on metal-catalyzed reactions, with applications including bulk chemicals production, natural product synthesis, pharmaceutical manufacturing, and materials preparation. In synthetic organic chemistry, C—C bond forming reactions through organopalladium catalysis is without contest the most widely used strategy. Examples include the Stille, Suzuki-Miyaura, Sonagashira, Negishi and Mizoroki-Heck reactions, but also the Pd(0)-catalyzed allylic alkylation also referred to as the Tsuji-Trost reaction. This latter example is well represented in natural product synthesis, where it enables a stereoselective approach to Csp³-Csp³ coupling through asymmetric allylic alkylation.

One aspect of all catalytic reactions is the generation of an active catalyst from stable precursor compounds. In many synthetic applications involving homogeneous organometallic catalysis, this is achieved through in situ combinations of supporting ligands and a metal source that are expected to assemble into an active form. While operationally convenient, this approach often leads to inefficient catalyst generation, which can negatively impact activity, reproducibility, and/or selectivity in the desired chemistry. An alternative strategy is to create and employ single-component precatalysts. These compounds are designed to already contain the required supporting ligands, along with carefully chosen reactive sites that lead to rapid and complete activation under the reaction conditions.

Despite the success and wide-spread application of the Pd-catalyzed asymmetric allylic alkylation, single-component precatalysts are only rarely used for this chemistry. Instead, these reactions are nearly always performed by in situ generation of a chiral Pd complex, often using prolonged pre-treatment of an achiral Pd source (typically Pd₂dba₃ or [Pd(allyl)Cl]₂) with an excess of the chiral (and usually expensive) ligand to ensure complete metalation. In some cases, this metalation also requires heating for extended periods of time, which renders the process much less practical and potentially irreproducible. The stability of the achiral Pd sources, in particular Pd₂dba₃, is another aspect that raises a series of issues. Interestingly, several isolable complexes of phosphinooxazoline (PHOX) ligands to Pd(0) and Pd(II) have been reported in the literature; however, while these are often the subjects of structural and/or mechanistic studies, they have been rarely used as single-component catalysts.

In stark contrast to the number of PHOX complexes known, accessing isolable Pd complexes of the privileged chiral diamide bisphosphine ligand platform—known in the art as the Trost ligands—has proven particularly challenging. Early work from those in the art established that mixing (S,S)-1,2-diaminocyclohexane-N,N′bis(2-diphenylphosphinobenzoyl) (L1) with Pd₂dba₃ generates a species consistent with [L1]Pd(dba), exhibiting a κ²—(P,P) coordination mode; however, this species was never isolated. Furthermore, this compound rapidly oxidizes when exposed to air to generate the catalytically inactive bis(amidate) complex [PNNP]Pd, making it unsuitable as an isolated precatalyst. Others have reported a dipalladium(II) complex with the prototype Trost ligand (L1), where each Pd center is coordinated by one phosphine and one carbonyl oxygen ([L1]Pd₂(allyl)₂(OTf)₂). While this compound is catalytically active, it was shown to give essentially racemic product during an attempted kinetic resolution of (±)-cyclopent-2-en-1-yl pivalate with NaCH(CO₂Me)₂, likely due to the κ²—(P,O) rather than κ²—(P,P) coordination mode. A compound of the empirical formula [L1]Pd(allyl)(OTf) can be isolated when using a 1:1 stoichiometry of Trost ligand to [Pd(allyl)(MeCN)₂][OTf]; however, this material exhibits a complex concentration-dependent solution behavior, generating multiple Pd-containing species including [L1]Pd₂(allyl)₂(OTf)₂ and higher oligomers.

Disclosed herein are embodiments of a new and versatile donor atom-based Pd(0) precatalyst. These new precatalyst embodiments are stable and isolable chiral Pd(0) precatalysts that can be used for asymmetric/entantioselective reactions, including asymmetric allylation reactions, like asymmetric allylic alkylations. The disclosed catalyst embodiments are well defined and monomeric, are easily handled without the need for an inert atmosphere or glovebox, and are highly effective for a multitude of asymmetric allylic alkylation reactions as described herein.

### III. Precatalyst Embodiments

Disclosed herein are embodiments of a chiral Pd(0) precatalyst. In particular embodiments, the chiral Pd(0) precatalyst has a structure according to Formula I, illustrated below.

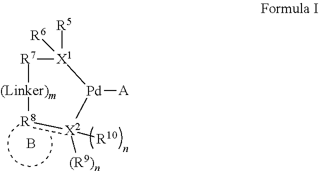

With reference to Formula I, the substituent recitations described below can apply.

The A group is a compound comprising at least one olefin moiety capable of coordinating with Pd(0). In an independent embodiment, the A group is not (1 E,4E)-1,5-diphenylpenta-1,4-dien-3-one or methyl (E)-2-acetoxy-3,5-diphenylpent-4-enoate.

Each of X¹ and X² independently are selected from phosphorus or nitrogen, provided that if one of X¹ or X² is nitrogen, then the other of X¹ or X² is phosphorus and is not nitrogen.

Each of R⁵ and R⁶ independently is selected from aliphatic, heteroaliphatic, haloaliphatic, haloheteroaliphatic, aromatic, or an organic functional group. In some embodiments, each R⁵ and R⁶ independently is selected from lower alkyl (e.g., methyl; ethyl; propyl; isopropyl; butyl; iso-butyl; tert-butyl; sec-butyl; pentyl and any isomers or cyclic versions thereof; hexyl and any isomers or cyclic versions thereof; heptyl and any isomers or cyclic versions thereof; octyl and any isomers or cyclic versions thereof; nonyl and any isomers or cyclic versions thereof; decyl and any isomers or cyclic versions thereof); aryl (e.g., phenyl, naphthyl, binaphthyl, 2,6-dimethoxy-1,1′-biphenyl, 2,6-diisopropoxy-1,1′-biphenyl, 2,4,6-triisopropyl-1,1′-biphenyl, 2,4,6-triisopropyl-2′,6′-dimethoxy-1,1′-biphenyl, 2′,4′,6′-triisopropyl-2,3,4,5-tetramethyl-1,1′-biphenyl, 3,5-(CF₃)-phenyl, 4-OMe-phenyl, 4-Cl-phenyl, oxydibenzene; and the like); heteroaryl (e.g., pyridinyl, pyrimidinyl, 1′,3′,5′-triphenyl-1′H-1,4′-bipyrazole, and the like); alkoxy, aroxy; amine; and cyclopentyldiene.

R⁷ is an aromatic group, an aliphatic group, a haloaliphatic group, a haloheteroaliphatic group, or a heteroaliphatic group. In some embodiments, R⁷ is aryl, heteroaryl, alkyl, haloalkyl, haloheteroalkyl, or heteroalkyl.

The linker, if present (such as when m is 1), comprises a chiral heteroaliphatic group having a formula

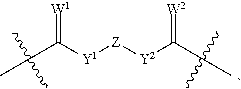

wherein Z is an aliphatic group, an aromatic group, or an organic functional group, wherein the aliphatic, aromatic, or organic functional group comprises a chiral substituent and/or an asymmetric center; and each of W¹, W², Y¹, and Y² independently are selected from oxygen, sulfur, or NR wherein R is hydrogen, aliphatic, or aromatic. In particular embodiments, Z is a cycloalkyl group (e.g., cyclopentyl, cyclohexyl, cycloheptyl, or the like), an alkyl group substituted with one or more aryl groups (e.g., a diarylethane group), or organic functional group that is a multicyclic group comprising aromatic and aliphatic groups (e.g., a 9,10-dihydro-9,10-ethanoanthracene group). In particular embodiments, each of W¹ and W² is oxygen. In particular embodiments, each of Y¹, and Y² independently is NR wherein R is hydrogen, alkyl, aryl, or heteroaryl.

If each n is 1, then R⁸ is an aromatic group, an aliphatic group, a haloaliphatic group, a haloheteroaliphatic group, or a heteroaliphatic group and is bound to X² via a single bond and no ring B is formed, which is illustrated in Formula I by way of the dashed curved line. In particular such embodiments, R⁸ is aryl, heteroaryl, alkyl, haloalkyl, haloheteroalkyl, or heteroalkyl. In other embodiments, if each n is 0, then R⁸ is a carbon atom that forms the ring B with X² and is bound to X² via a double bond, wherein the ring B can be an aromatic ring or a heterocyclic ring, wherein any such aromatic ring or heterocyclic ring comprises a chiral substituent or an asymmetric center. In such embodiments, the dashed curved line is present and represents the ring structure of ring B. In particular such embodiments, R⁸ forms a four-membered, five-membered, six-membered, or seven-membered aromatic or heterocyclic ring B with X².

Each of R⁹ and R¹⁰, if present (such as when n is 1), independently is selected from aliphatic, heteroaliphatic, haloaliphatic, haloheteroaliphatic, aromatic, or an organic functional group. In some such embodiments, each R⁹ and R¹⁰ independently is selected from lower alkyl (e.g., methyl; ethyl; propyl; isopropyl; butyl; iso-butyl; tert-butyl; sec-butyl; pentyl and any isomers or cyclic versions thereof; hexyl and any isomers or cyclic versions thereof; heptyl and any isomers or cyclic versions thereof; octyl and any isomers or cyclic versions thereof; nonyl and any isomers or cyclic versions thereof; decyl and any isomers or cyclic versions thereof); aryl (e.g., phenyl, naphthyl, binaphthyl, 2,6-dimethoxy-1,1′-biphenyl, 2,6-diisopropoxy-1,1′-biphenyl, 2,4,6-triisopropyl-1,1′-biphenyl, 2,4,6-triisopropyl-2′,6′-dimethoxy-1,1′-biphenyl, 2′,4′,6′-triisopropyl-2,3,4,5-tetramethyl-1,1′-biphenyl, 3,5-(CF₃)-phenyl, 4-OMe-phenyl, 4-Cl-phenyl, oxydibenzene; and the like); heteroaryl (e.g., pyridinyl, pyrimidinyl, 1′,3′,5′-triphenyl-1′H-1,4′-bipyrazole, and the like); alkoxy, aroxy; amine; and cyclopentyldiene.

Each n is 1 or 0; and m is 1 or 0.

In some embodiments, the A group is a cyclic or acyclic compound comprising at least one olefin moiety that coordinates with the Pd(0). In particular embodiments, the A group has a structure according to Formula II, illustrated below.

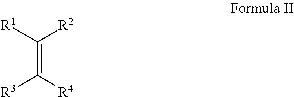

With reference to Formula II, each of R¹, R², R³, and R⁴ independently is (i) selected from hydrogen, aliphatic, aromatic, or an electron-withdrawing group provided that at least one of R¹, R², R³, or R⁴ is other than hydrogen; or (ii) R¹ and R³, or R² and R⁴ join together to provide a cyclic group, such as a 5- to 7-membered cyclic group, and the remaining R¹ and R³ groups, or R² and R⁴ groups are hydrogen. In particular embodiments, the A group is maleic anhydride (wherein, with reference to Formula II, R¹ and R³ are hydrogen and R² and R⁴ join together to provide the 5-membered anhydride) or a maleimide (wherein, with reference to Formula II, R¹ and R³ are hydrogen and R² and R⁴ join together to provide the 5-membered maleimide). In some embodiments, the maleimide can be a protected maleimide wherein the nitrogen atom of the maleimide group is bound to a protecting group, such as an aliphatic group or an aromatic group. In other embodiments, the A group has a structure wherein at least one of R¹, R², R³, and R⁴ (e.g., wherein one, two, three, or four of R¹, R², R³, and R⁴) are an electron-withdrawing group, such as an ester group, a carboxyl group, a cyano group, an aldehyde group, a ketone group, or a nitro group, and any remaining R¹, R², R³, or R⁴ groups are hydrogen or phenyl. In some embodiments, the A group has a structure wherein R¹ is an ester or cyano, and each of R², R³, and R⁴ is hydrogen. In yet additional embodiments, the A group has a structure wherein one of R¹, R², R³, or R⁴ is an alkenyl group so as to provide a diene-containing compound comprising at least two olefin moieties that can be conjugated or unconjugated. Such diene-containing compounds can be cyclic or acyclic. In representative embodiments, the A group can be selected from any of the compounds illustrated in Table A, below.

TABLE A

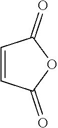

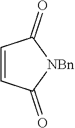 [SMILES: O=C1C=CC(=O)N1Cc1ccccc1]

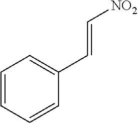 [SMILES: O=[N+]([O-])/C=C/c1ccccc1]

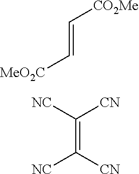

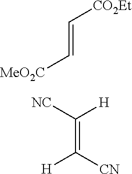

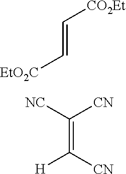

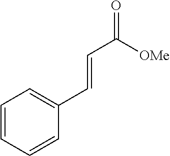 [SMILES: COC(=O)/C=C/c1ccccc1]

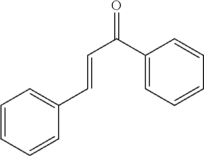 [SMILES: O=C(/C=C/c1ccccc1)c1ccccc1]

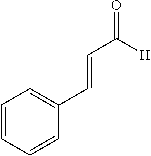

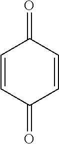

In some embodiments, the Pd(0) precatalyst can have a structure according to Formulas Ill or IV.

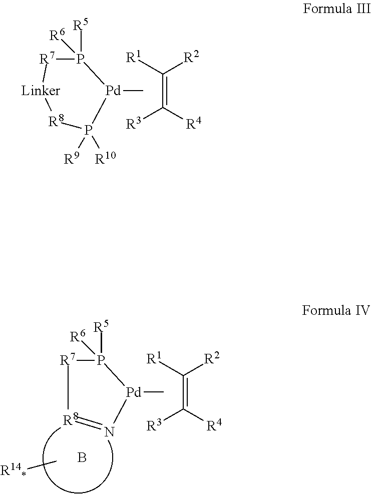

With reference to Formula III, the substituent recitations provided below can apply.

Each of R¹, R², R³, and R⁴ are as recited for Formula II.

Each of R⁵, R⁶, R⁹, and R¹⁰ independently is as recited for Formula I. In particular embodiments, each R⁵, R⁶, R⁹, and R¹⁰ is selected from aliphatic, heteroaliphatic, haloaliphatic, haloheteroaliphatic, aryl, heteroaryl, or an organic functional group. In particular embodiments, each of R⁵, R⁶, R⁹, and R¹⁰ independently is selected from aliphatic, heteroaliphatic, haloaliphatic, haloheteroaliphatic, aromatic, or an organic functional group. In some such embodiments, each R⁹ and R¹⁰ independently is selected from lower alkyl (e.g., methyl; ethyl; propyl; isopropyl; butyl; iso-butyl; tert-butyl; sec-butyl; pentyl and any isomers or cyclic versions thereof; hexyl and any isomers or cyclic versions thereof; heptyl and any isomers or cyclic versions thereof; octyl and any isomers or cyclic versions thereof; nonyl and any isomers or cyclic versions thereof; decyl and any isomers or cyclic versions thereof); aryl (e.g., phenyl, naphthyl, binaphthyl, 2,6-dimethoxy-1,1′-biphenyl, 2,6-diisopropoxy-1,1′-biphenyl, 2,4,6-triisopropyl-1,1′-biphenyl, 2,4,6-triisopropyl-2′,6′-dimethoxy-1,1′-biphenyl, 2′,4′,6′-triisopropyl-2,3,4,5-tetramethyl-1,1′-biphenyl, 3,5-(CF₃)-phenyl, 4-OMe-phenyl, 4-Cl-phenyl, oxydibenzene; and the like); heteroaryl (e.g., pyridinyl, pyrimidinyl, 1′,3′,5′-triphenyl-1′H-1,4′-bipyrazole, and the like); alkoxy, aroxy; amine; and cyclopentyldiene.

Each of R⁷ and R⁸ independently is as recited for Formula I. In particular embodiments, each of R⁷ and R⁸ independently is an aromatic group, an aliphatic group, a haloaliphatic group, a haloheteroaliphatic group, or a heteroaliphatic group. In particular embodiments, each of R⁷ and R⁸ independently is an aryl group, a heteroaryl group, an aliphatic group, a haloaliphatic group, a haloheteroaliphatic group, or a heteroaliphatic group. In particular embodiments, each of R⁷ and R⁸ independently is aryl, heteroaryl, alkyl, haloalkyl, haloheteroalkyl, or heteroalkyl.

The linker is as recited for Formula I. In particular embodiments, the linker has a structure according to a formula

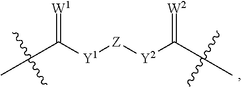

wherein Z is a cyclic aliphatic, cyclic heteroaliphatic group, an aryl group, or a heteroaryl group, wherein any such group comprises a chiral substituent or an asymmetric center; each of W¹ and W² is oxygen; and each of Y¹ and Y² is NH. In particular embodiments, Z is a cycloalkyl group (e.g., cyclopentyl, cyclohexyl, cycloheptyl, or the like), an alkyl group substituted with one or more aryl groups (e.g., a diarylethane group), or organic functional group that is a multicyclic group comprising aromatic and aliphatic groups (e.g., a 9,10-dihydro-9,10-ethanoanthracene group). In particular embodiments, each of W¹ and W² is oxygen. In particular embodiments, each of Y¹, and Y² independently is NR wherein R is hydrogen, alkyl, aryl, or heteroaryl.

With reference to Formula IV, the substituent recitations provided below can apply.

Each of R¹, R², R³, and R⁴ are as recited for Formula II.

Each of R⁵ and R⁶ independently is as recited for Formula I. In particular embodiments, each R⁵ and R⁶ is selected from aliphatic, heteroaliphatic, haloaliphatic, haloheteroaliphatic, aryl, heteroaryl, or an organic functional group. In particular embodiments, each of R⁵ and R⁶ independently is selected from aliphatic, heteroaliphatic, haloaliphatic, haloheteroaliphatic, aromatic, or an organic functional group. In some such embodiments, each R⁹ and R¹⁰ independently is selected from lower alkyl (e.g., methyl; ethyl; propyl; isopropyl; butyl; iso-butyl; tert-butyl; sec-butyl; pentyl and any isomers or cyclic versions thereof; hexyl and any isomers or cyclic versions thereof; heptyl and any isomers or cyclic versions thereof; octyl and any isomers or cyclic versions thereof; nonyl and any isomers or cyclic versions thereof; decyl and any isomers or cyclic versions thereof); aryl (e.g., phenyl, naphthyl, binaphthyl, 2,6-dimethoxy-1,1′-biphenyl, 2,6-diisopropoxy-1,1′-biphenyl, 2,4,6-triisopropyl-1,1′-biphenyl, 2,4,6-triisopropyl-2′,6′-dimethoxy-1,1′-biphenyl, 2′,4′,6′-triisopropyl-2,3,4,5-tetramethyl-1,1′-biphenyl, 3,5-(CF₃)-phenyl, 4-OMe-phenyl, 4-Cl-phenyl, oxydibenzene; and the like); heteroaryl (e.g., pyridinyl, pyrimidinyl, 1′,3′,5′-triphenyl-1′H-1,4′-bipyrazole, and the like); alkoxy, aroxy; amine; and cyclopentyldiene.

R⁷ is as recited for Formula I. In particular embodiments, R⁷ is an aromatic group, an aliphatic group, a haloaliphatic group, a haloheteroaliphatic group, or a heteroaliphatic group. In particular embodiments, R⁷ is an aryl group, a heteroaryl group, an aliphatic group, a haloaliphatic group, a haloheteroaliphatic group, or a heteroaliphatic group. In particular embodiments, R⁷ is aryl, heteroaryl, alkyl, haloalkyl, haloheteroalkyl, or heteroalkyl.

R⁸ is as recited for Formula I. In particular embodiments, R⁸ is a carbon atom and forms a four-, five-, or six-membered heterocyclic ring system, or a five- or 6-membered heteroaryl ring system together with the N atom to which it is bound, wherein the heterocyclic, aryl, or heteroaryl ring systems comprise a chiral substituent or an asymmetric center as represented by the * symbol, which indicates that either R¹⁴ is a chiral substituent or is attached to an asymmetric carbon atom and wherein R¹⁴ is selected from aliphatic, heteroaliphatic, aromatic, or an organic functional group.

Compounds having structures of Formula III can further have a structure according to any one of Formulas IIIA, IIIB, or IIIC, shown below.

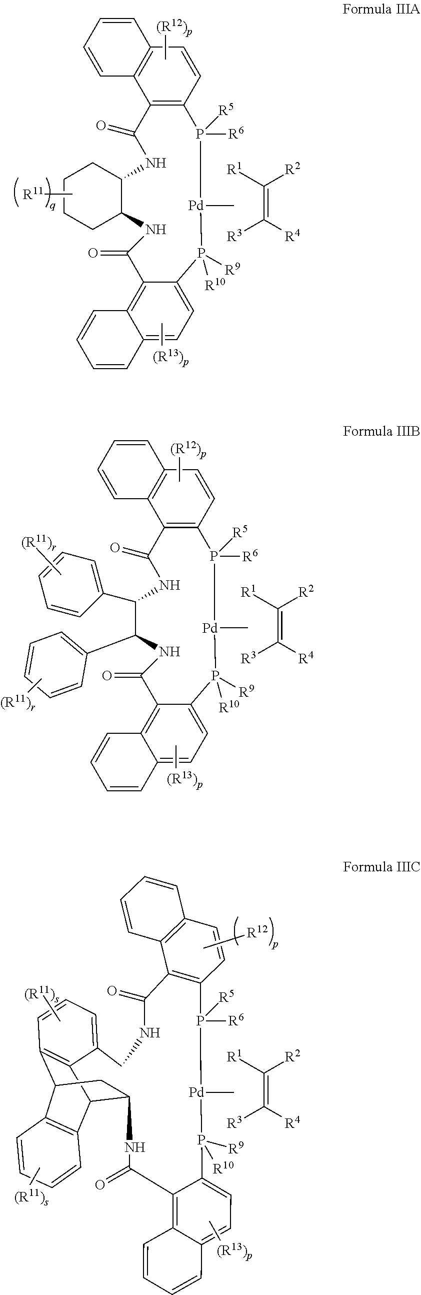

With reference to Formulas IIIA-IIIC, the substituent recitations provided below can apply and the dashed ( [symbol] ) bond is used to represent an optional bond.

Each of R¹, R², R³, and R⁴ are as recited for Formula II.

Each of R⁵, R⁶, R⁹, and R¹⁰ independently is as recited for Formulas I or Ill.

Each R¹¹ independently is selected from aliphatic, heteroaliphatic, haloaliphatic, aromatic, or an organic functional group. In particular embodiments, each R¹¹ independently is selected from alkyl, alkenyl, alkynyl, heteroalkyl, heteroalkenyl, heteroalkynyl, haloalkyl, aryl, heteroaryl, or a particular organic functional group as defined herein.

Each of R¹² and R¹³ independently, for each occurrence, is selected from aliphatic, heteroaliphatic, haloaliphatic, aromatic, or an organic functional group. In particular embodiments, each of R¹² and R¹³ independently, for each occurrence, is selected from alkyl, alkenyl, alkynyl, heteroalkyl, heteroalkenyl, heteroalkynyl, haloalkyl, aryl, heteroaryl, or a particular organic functional group as defined herein.

Each p independently is an integer selected from 0 to 6, such as 0, 1, 2, 3, 4, 5, or 6; q is an integer ranging from 0 to 10, such as 0, 1, 2, 3, 4, 5, 6, 7, 8, 9, or 10; each r independently is an integer selected from 0 to 5, such as 1, 2, 3, 4, or 5; and each s independently is an integer selected from 0 to 4, such as 1, 2, 3, or 4.

Compounds having structures of Formula IV can further have a structure according to Formula IVA or Formula IVB.

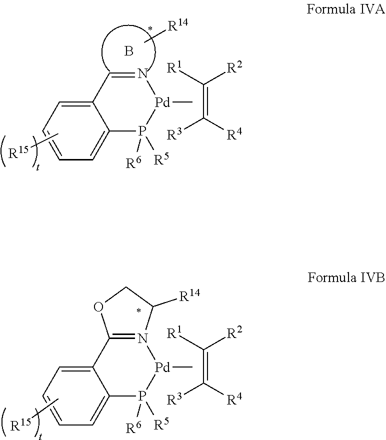

With reference to Formulas IVA and IVB, the substituent recitations provided below can apply and the * symbol represents that the R¹⁴ group is a chiral group or bound to an asymmetric carbon of Ring B.

Each of R¹, R², R³, and R⁴ are as recited for Formula II.

Each of R⁵ and R⁶ independently is as recited for any of Formulas I, II, IIIA, IIIB, or IIIC.

Ring B is as recited for Formula I. In particular embodiments, Ring B is a five- or six-membered heterocyclic or heteroaryl ring system and comprises an asymmetric carbon that bears the R¹⁴ substituent.

R¹⁴ is selected from aliphatic, heteroaliphatic, aromatic, or an organic functional group and is chiral or is bound to an asymmetric center of Ring B. In particular embodiments, R¹⁴ is selected from alkyl, alkenyl, alkynyl, heteroalkyl, heteroalkenyl, heteroalkynyl, aryl, heteroaryl, or a particular organic functional group as defined herein and R¹⁴ is bound to an asymmetric carbon atom of Ring B.

Each R¹⁵ independently is selected from aliphatic, heteroaliphatic, haloaliphatic, aromatic, or an organic functional group. In particular embodiments, each R¹⁵ independently is selected from alkyl, alkenyl, alkynyl, heteroalkyl, heteroalkenyl, heteroalkynyl, haloalkyl, aryl, heteroaryl, or a particular organic functional group as defined herein.

Variable t is an integer selected from 0 to 4, such as 1, 2, 3, or 4.

Representative precatalyst embodiments having structures according to one or more of Formulas I, Ill, IIIA-IIIC, IV, and/or IVA are illustrated below in Table B.

TABLE B

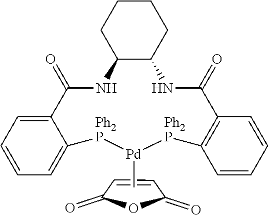

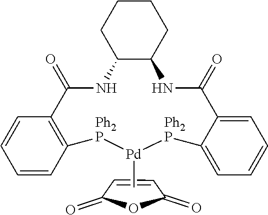

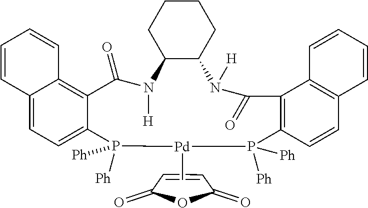

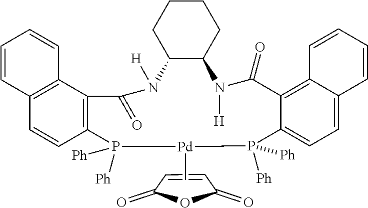

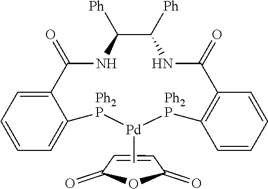

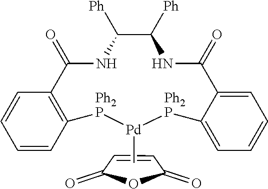

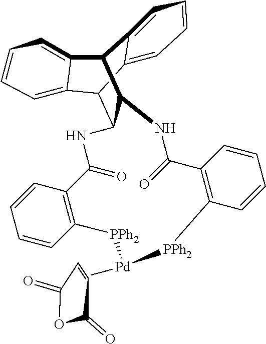

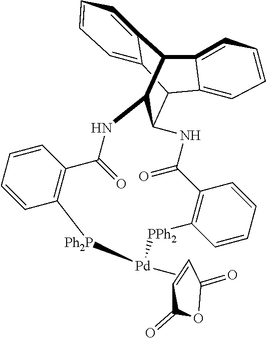

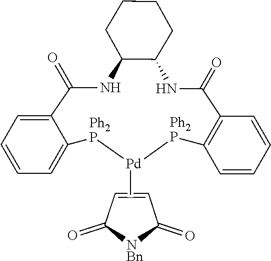

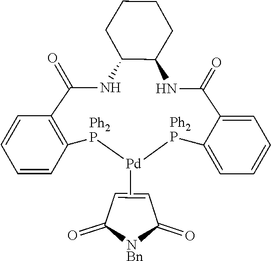

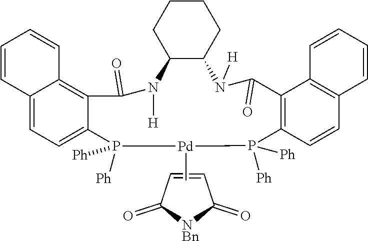

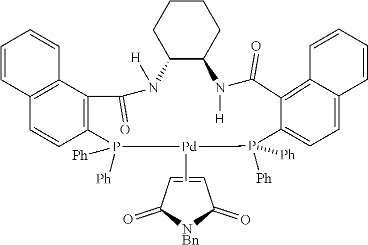

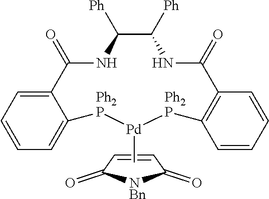

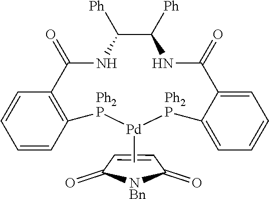

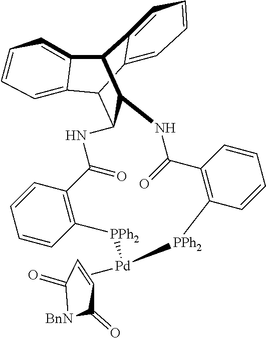

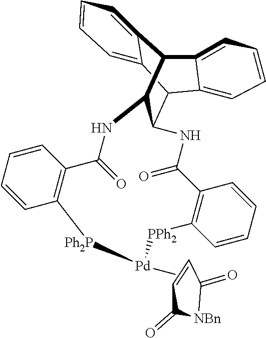

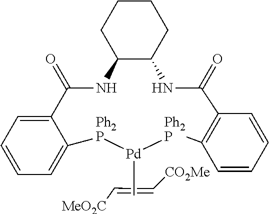

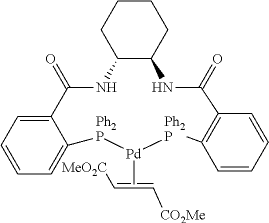

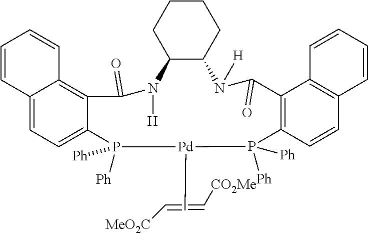

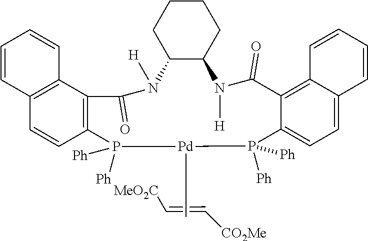

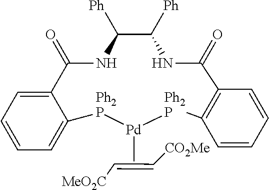

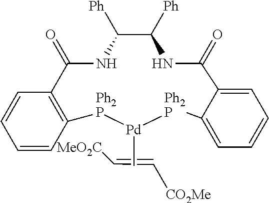

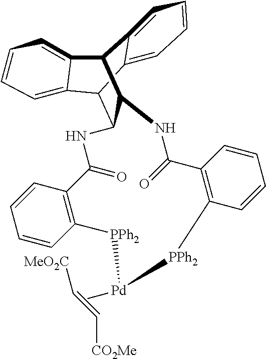

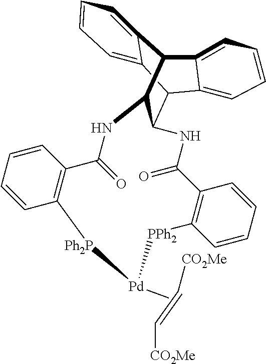

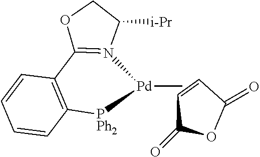

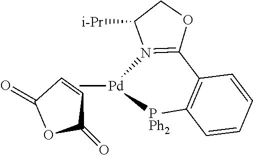

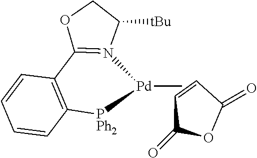

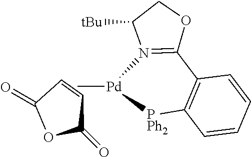

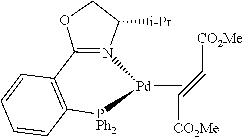

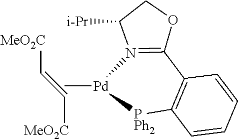

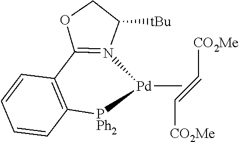

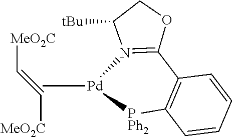

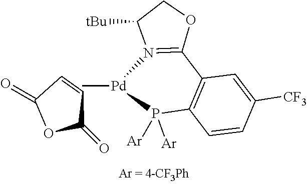

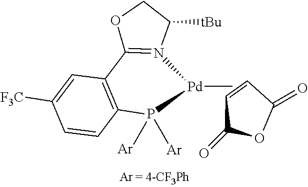

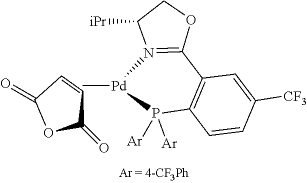

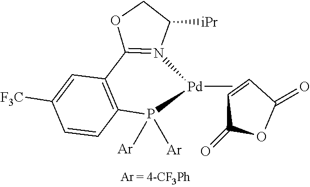

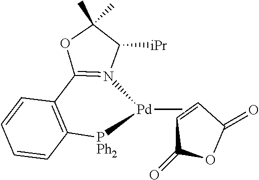

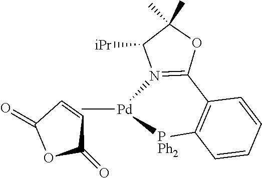

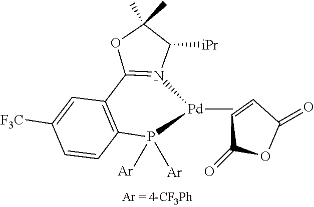

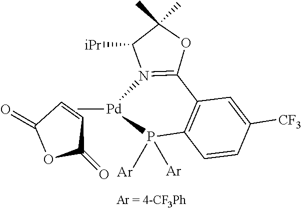

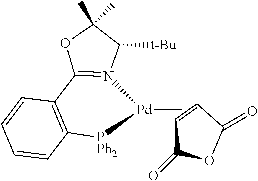

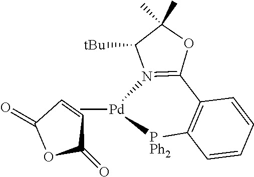

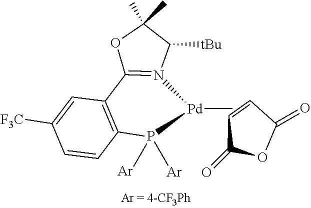

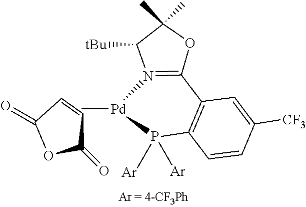

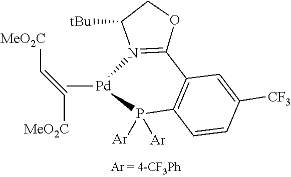

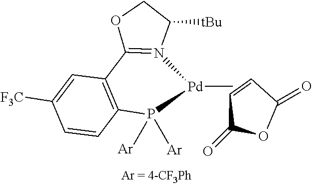

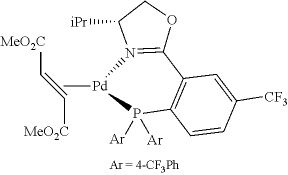

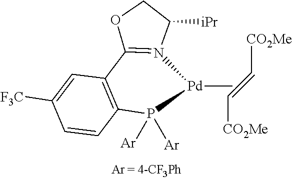

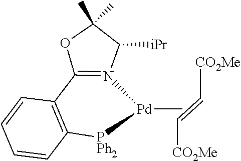

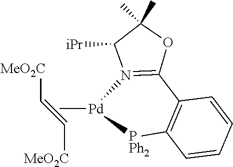

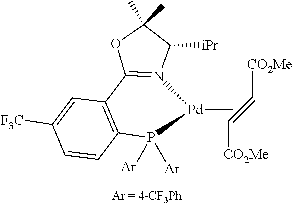

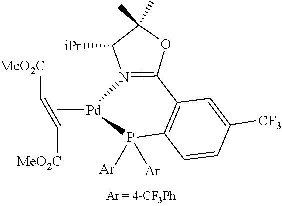

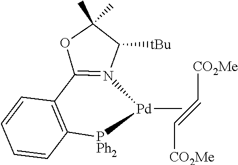

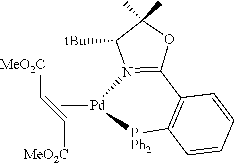

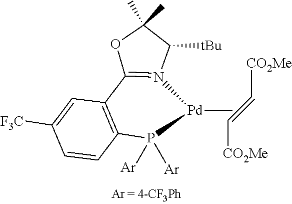

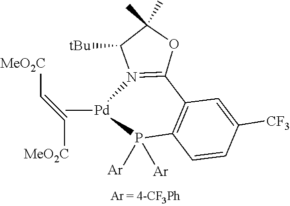

In independent embodiments, the precatalyst is not, or is other than, a compound according to the following compounds:

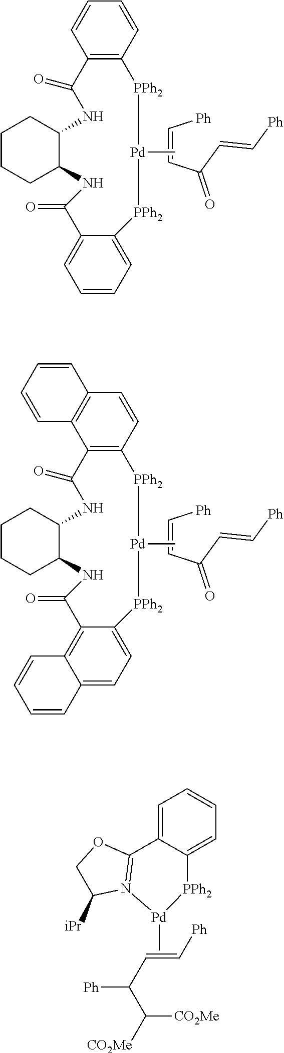

In other independent embodiments, the precatalyst is not, or is other than, a Pd(II) complex.

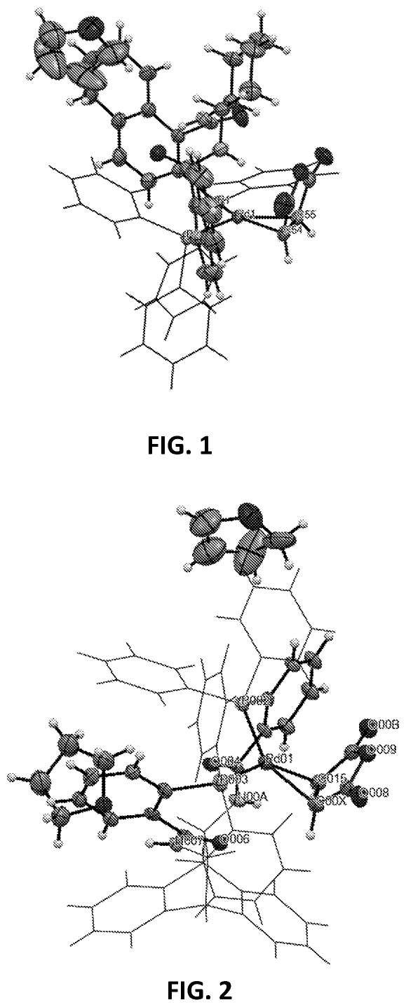

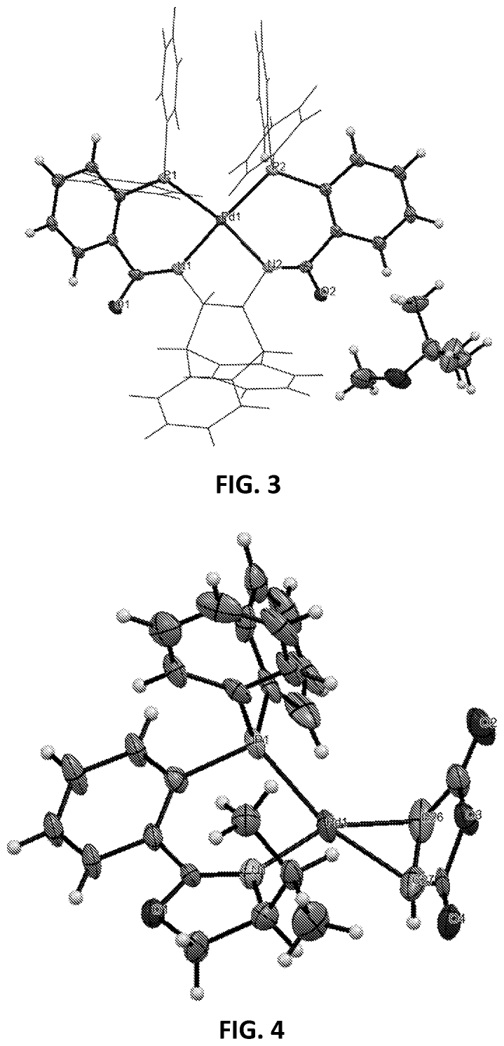

Without being limited by a particular theory, it currently is understood, based on the solid-state molecular structures of certain precatalyst embodiments (e.g., precatalysts 2 and 4 as described in the Examples), that two distinct conformations can exist with respect to the orientation of the chiral ligand backbone and the coordinated maleic anhydride. Exemplary solid-state molecular structures are shown in FIGS. 1-4. For example, in some embodiments (e.g., such as with precatalyst 2), the A group (e.g., maleic anhydride) binds with any carbonyl groups pointing toward the chiral tether of the donor atom-containing ligand moiety (referred to herein as an endo-like conformation). This orientation enables an intramolecular hydrogen bond between one of the amide N—H protons and a carbonyl oxygen from maleic anhydride. It currently is understood that this type of hydrogen bonding interaction between ligand and substrate can serve as a mechanism for stereoinduction in many reactions using the precatalyst (e.g., asymmetric allylic alkylation reactions). In some other embodiments (e.g., the ^{Ph}ANDEN precatalyst 4), a conformation can be observed wherein carbonyl groups of an A group (e.g., maleic anhydride oxygens) point away from the chiral tether (referred to herein as an exo-like conformation). In yet additional embodiments, such as embodiments using a precatalyst according to Formula IV (or IVA or IVB), the precatalyst can adopt a conformation where carbonyl groups of the A group (e.g., maleic anhydride oxygens) are endo with respect to the R¹⁴ group.

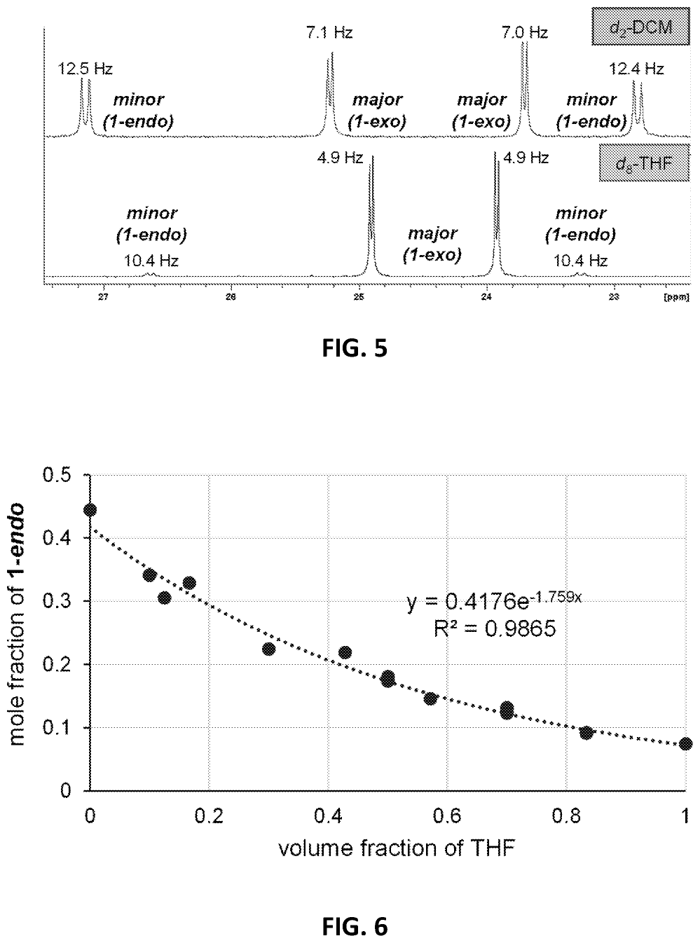

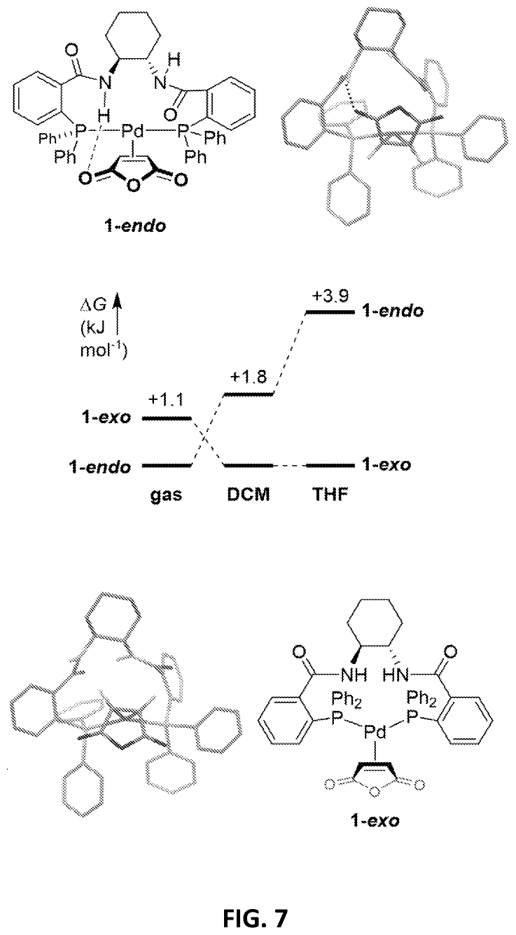

In some embodiments, solution-phase characterization of certain precatalysts (e.g., precatalysts 1-4) using multinuclear and multidimensional NMR spectroscopy can show the presence of two distinct species in each case. Solely by way of example, ³¹P NMR spectra obtained in either d₂-DCM or d₈-THF for precatalyst 1 showed two sets of signals, each of which is a matching pair of doublets that is characteristic of bidentate κ²—P,P coordination to Pd (FIG. 5). For this embodiment, a primary species exists in THF, with the second species only just visible above the spectrum baseline (˜14:1 ratio). In contrast, the two species have a 55:45 ratio in DCM. Without being limited to a single theory, it currently is understood that the two species are not evidence of a monomer/dimer equilibrium, and the two species can interconvert, as evidenced by changes in molar ratio as a function of solvent composition in a DCM/THF mixture (e.g., see FIGS. 6 and 7 which show results for precatalyst 1). For the embodiment shown in FIG. 6, as the volume fraction of THF in the sample increases, the amount of the minor species decreases exponentially, converging at the 14:1 ratio observed in 100% THF. In view of this behavior, it currently is understood that the two species in solution are endo and exo conformers, with the exo conformer being the major conformer in certain embodiments. This assignment of exo as the major species is based on extensive 2D NMR spectroscopy data.

### IV. Method Embodiments

Also disclosed herein are methods for making and using the donor atom-based Pd(0) precatalyst embodiments of the present disclosure.

In some embodiments, the chiral donor atom-based Pd(0) precatalyst can be made by converting a Pd(0) precursor complex to the donor atom-based Pd(0) precatalyst. Such method embodiments can comprise exposing the Pd(0) precursor complex, such as a complex having a structure according to Formula A, illustrated below (wherein the illustrated A group has a structure according to Formula II described herein and “Ar” represents an aromatic substituent), to a donor atom-containing ligand group comprising at least one phosphorus atom. In particular embodiments, the Pd(0) precursor complex is^{DMP}DAB-Pd-MAH, which has a structure as also illustrated below.

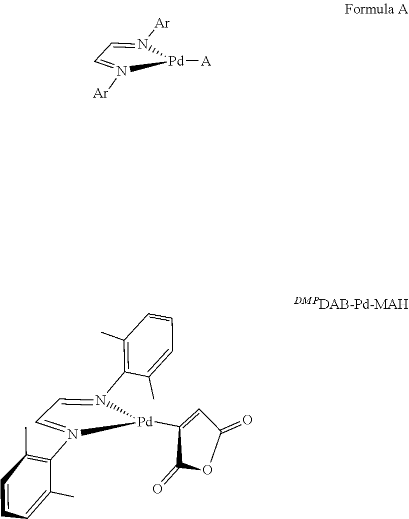

In particular embodiments, the donor atom-containing ligand group is a bidentate group comprising two phosphorus atoms capable of coordinating with palladium. In other embodiments, the donor atom-containing ligand group is a bidentate group comprising one phosphorus atom and one nitrogen atom, each of which is capable of coordinating with palladium. Exemplary donor atom-containing ligands can have a structure according to Formula V, below, wherein each of X¹, X², R⁵ to R¹⁰, ring B, the linker, m, and each n is as described herein for Formulas I, III, and/or IV (including any subgeneric formulas thereof provided herein). In particular embodiments, X¹ is phosphorus and X² is phosphorus or nitrogen.

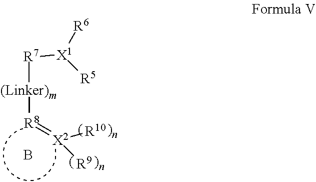

In particular embodiments, the donor atom-containing ligand can have a structure according to any of Formulas VI or VII, wherein the each of R⁵ to R¹⁰, R¹⁴, ring B, and the linker is as described herein for Formulas I, III, and/or IV (including any of Formulas IIIA-IIIC and/or IVA).

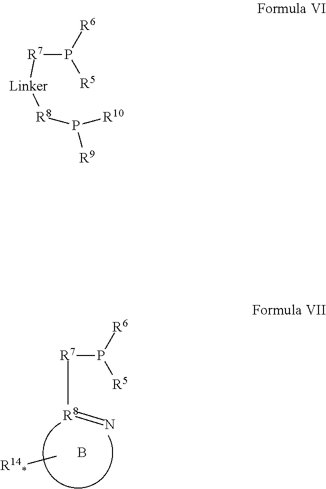

In yet additional embodiments, the donor atom-containing ligand can have a structure according to any of Formulas VIA-VIC or VIIA, wherein each of R⁵ to R¹³ is as described herein for Formulas I, III, and/or IV (including any of Formulas IIIA-IIIC and/or IVA).

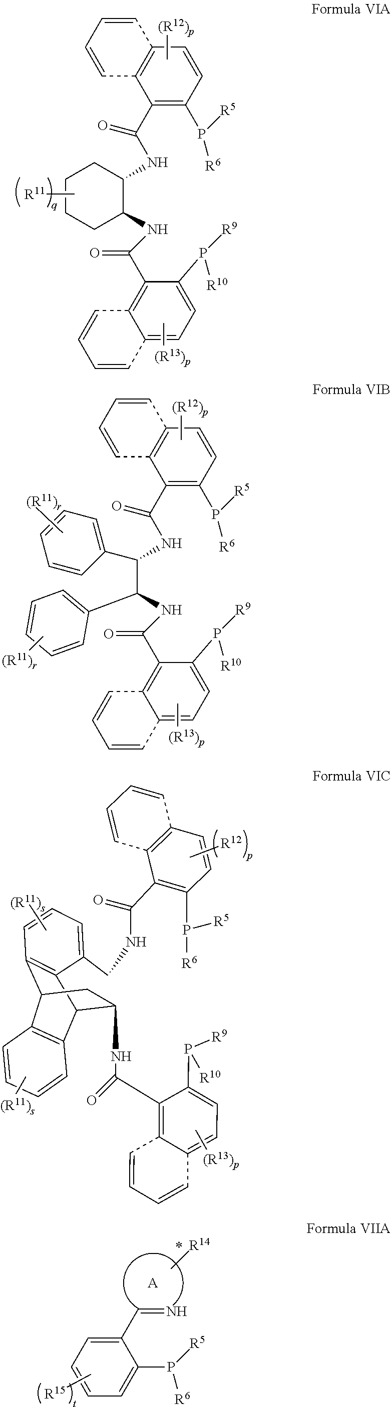

In some particular embodiments, the donor atom-containing ligand group is selected from the structures illustrated below in Table C.

TABLE C

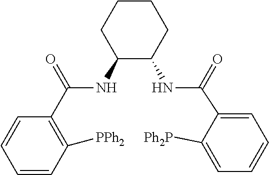

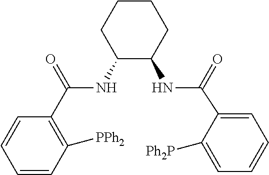

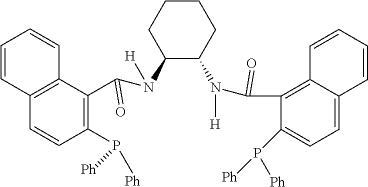

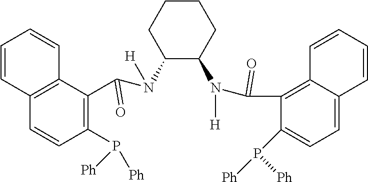

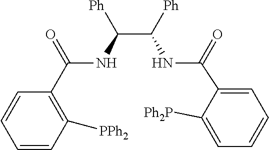

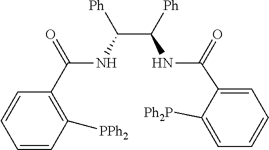

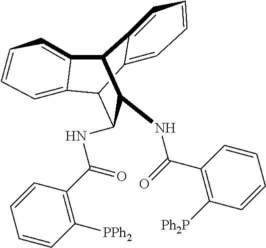

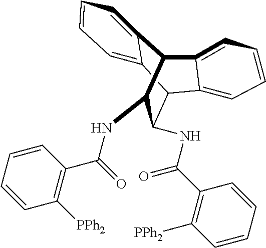

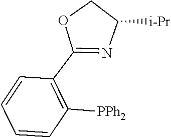

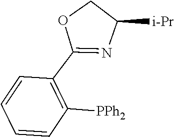

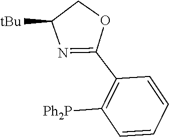

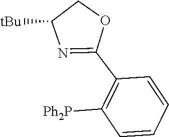

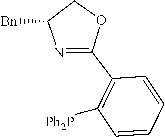

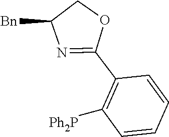

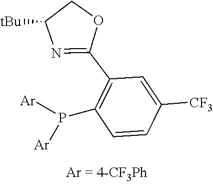

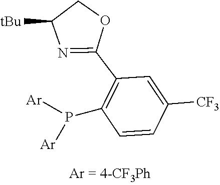

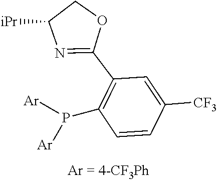

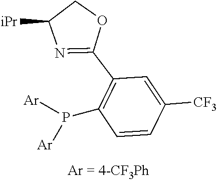

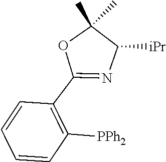

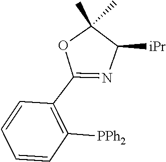

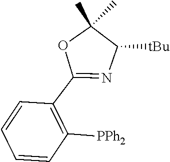

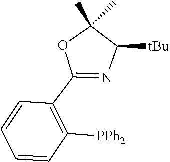

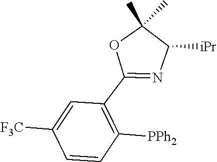

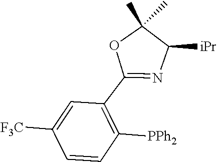

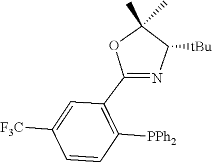

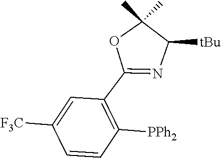

The Pd(0) precursor complex can be exposed to the donor atom-containing ligand group in the presence of a solvent (e.g., THF) and the resulting reaction mixture is allowed to mix for a suitable time period. The method can further comprise isolating the donor atom-based Pd(0) precatalyst by removing the THF solvent and triturating the resulting solid with a different solvent (e.g., hexanes, pentane, diethyl ether, or any combination of these) and then decanting the liquid phase. This can be repeated one or more times until any excess reactants are removed. The desired product can be isolated as a solid after removing any remaining solvent. Alternatively, the Pd(0) catalyst can be isolated and purified by crystallization from the reaction solvent (e.g. THF) using an appropriate anti-solvent (e.g. hexanes, pentane, diethyl ether, or any combination of these). Filtration to collect the solid and washing with anti-solvent affords the desired product. Such method embodiments provide rapid conversion to the corresponding donor atom-based Pd(0) catalyst, even at ambient temperature, with minimal to no side-product formation.

The disclosed Pd(0) precatalyst embodiments be used in multiple different applications. For example, the Pd(0) precatalyst embodiments can be used for Pd-catalyzed enantioselective chemical reactions. In particular embodiments, Pd(0) precatalyst embodiments according to the present disclosure can be used to catalyze myriad different enantioselective Pd-catalyzed chemical reactions, such as Pd-mediated allylations. Exemplary such methods are described below and are provided solely as non-limiting examples.

The Pd(0) precatalyst embodiments disclosed herein can be used in asymmetric allylic alkylations. In such embodiments, the Pd(0) precatalyst embodiments can be used as a chiral catalyst to facilitate carbon-carbon bond forming reactions and/or carbon-heteroatom forming reactions (e.g., carbon-oxygen, carbon-sulfur, or carbon-nitrogen bond formation), and/or between a racemic alkene-containing starting material and a racemic nucleophile. The racemic alkene-containing starting material can be cyclic or acyclic. The racemic nucleophile can be selected from carbon-based nucleophiles, such as malonate nucleophiles, β-keto ester nucleophiles, nitrosulfonyl nucleophiles, nitroalkane nucleophiles, and the like; oxygen-based nucleophiles, such as alcohol nucleophiles, carboxylate nucleophiles, hydrogencarbonate nucleophiles, and the like; nitrogen-based nucleophiles, such as alkylamine nucleophiles, azide nucleophiles, sulfonamide nucleophiles, imide nucleophiles, and the like; or sulfur-based nucleophiles, such as sodium benzenesulfinate nucleophiles. In particular such embodiments, the method comprises exposing a racemic nucleophile precursor compound to a suitable base (e.g., a base capable of deprotonating the nucleophile) and then combining the formed nucleophile with the racemic alkene-containing starting material and the Pd(0) precatalyst; however, the components of the method can be added in any suitable order as would be recognized by those skilled in the art, particularly with the benefit of the present disclosure. In some embodiments, the Pd(0) precatalyst can be generated in situ during the method by combining a donor atom-based ligand as described above with an embodiment of the Pd(0) precursor complex described above. In representative examples of such methods,

In yet additional embodiments, the Pd(0) precatalyst embodiments can be used in desymmetrization reactions of meso compounds. In such embodiments, the method can comprise reacting a meso starting material with a racemic nucleophile (such as any such nucleophiles described herein) in the presence of a Pd(0) precatalyst embodiment of the present disclosure. In some embodiments, the Pd(0) precatalyst is produced in situ during the method by combining a donor atom-based ligand as described above with an embodiment of the Pd(0) precursor complex described above.

In yet further embodiments, the Pd(0) precatalyst embodiments of the present disclosure can be used in decarboxylative asymmetric allylic alkylations. In such method embodiments, a racemic allyl carbonate compound is reacted with a Pd(0) precatalyst embodiment of the present disclosure to facilitate migration of the allyl group to another position on the starting material, thereby forming a new carbon-carbon bond in an enantioselective fashion.

Additional methods in which the disclosed Pd(0) precatalysts can be used can include, but are not limited, to direct allylations of prochiral heterocyclic starting materials, allylations of enol silane heterocyclic starting materials, and/or allylation of heterocycles via decarboxylation.

While the above-described method embodiments are illustrative of methods in which the disclosed Pd(0) precatalysts can be used, they are not intended as an exhaustive list.

In any or all of the above-described method embodiments, the amount of the Pd(0) precursor complex can range from 0.001 mol % to 40 mol %, such as 0.01 mol % to 15 mol %, or 0.1 mol % to 5 mol %. In some embodiments, the donor atom-containing ligand can be added in an amount so as to provide 1 to 2 (e.g., 1, 1.2, 1.5, or 1.75) mol equivalents relative to the Pd(0) precursor complex.

### V. Overview of Several Embodiments

1. A chiral precatalyst having a structure according to Formula I

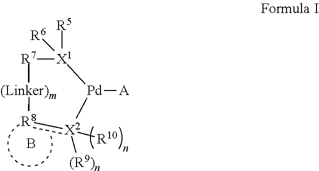

wherein:
the Pd atom has an oxidation state of 0;
the A group is a compound comprising at least one olefin moiety capable of coordinating with Pd(0), provided that the A group is not (1 E,4E)-1,5-diphenylpenta-1,4-dien-3-one or methyl (E)-2-acetoxy-3,5-diphenylpent-4-enoate;
each of X¹ and X² independently are selected from phosphorus or nitrogen, provided that if one of X¹ or X² is nitrogen, then the other of X¹ or X² is phosphorus and is not nitrogen;
each of R⁵ and R⁶ independently is selected from aliphatic, heteroaliphatic, haloaliphatic, haloheteroaliphatic, aromatic, or an organic functional group;
R⁷ is an aromatic group, an aliphatic group, a haloaliphatic group, a haloheteroaliphatic group, or a heteroaliphatic group;
the linker, if present, comprises a chiral heteroaliphatic group having a formula

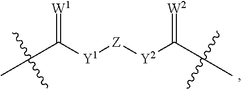

wherein Z comprises an aliphatic group, an aromatic group, or an organic functional group, wherein the aliphatic, aromatic, or organic functional group comprises a chiral substituent and/or an asymmetric center; and each of W¹, W², Y¹, and Y² independently are selected from oxygen, sulfur, or NR wherein R is hydrogen, aliphatic, or aromatic;
each of R⁹ and R¹⁰, if present, independently is selected from aliphatic, heteroaliphatic, haloaliphatic, haloheteroaliphatic, aromatic, or an organic functional group;
each n is 1 or each n is 0;
m is 1 or 0; and
(i) if each n is 1, then R⁸ is an aromatic group, an aliphatic group, a haloaliphatic group, a haloheteroaliphatic group, or a heteroaliphatic group and is bound to X² via a single bond and no ring B is formed; or
(ii) if each n is 0, then R⁸ is a carbon atom that forms the ring B with X² and is bound to X² via a double bond, wherein the ring B is an aromatic or heterocyclic ring comprising a chiral substituent or an asymmetric center; and
provided that for Formula I, the compound is not or is other than

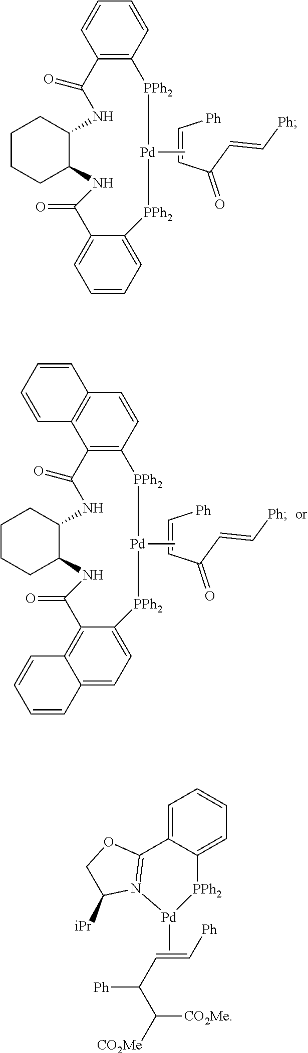

In any or all embodiments, the A group has a Formula II

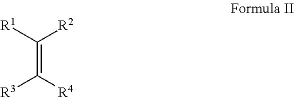

wherein:
each of R¹, R², R³, and R⁴ independently is (i) selected from hydrogen, aliphatic, aromatic, or an electron-withdrawing group provided that at least one of R¹, R², R³, or R⁴ is other than hydrogen; or (ii) R¹ and R³, or R² and R⁴ join together to provide a cyclic group and the remaining R¹ and R³ groups, or R² and R⁴ groups are hydrogen.

In any or all of the above embodiments, the A group has a structure as illustrated in Table A herein.

In any or all of the above embodiments, the chiral precatalyst has a structure according to Formula III

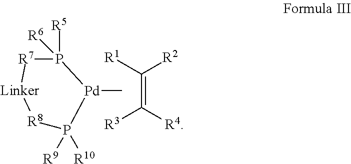

In any or all of the above embodiments, Z is selected from a cyclohexyl group, a diarylethane group, or a 9,10-dihydro-9,10-ethanoanthracene group; each of Y¹ and Y² is NH; and each of W¹ and W² is oxygen.

In any or all of the above embodiments, each of R⁵, R⁶, R⁹, and R¹⁰ is phenyl.

In any or all of the above embodiments, the chiral precatalyst has a structure according to any one of Formulas IIIA-IIIC

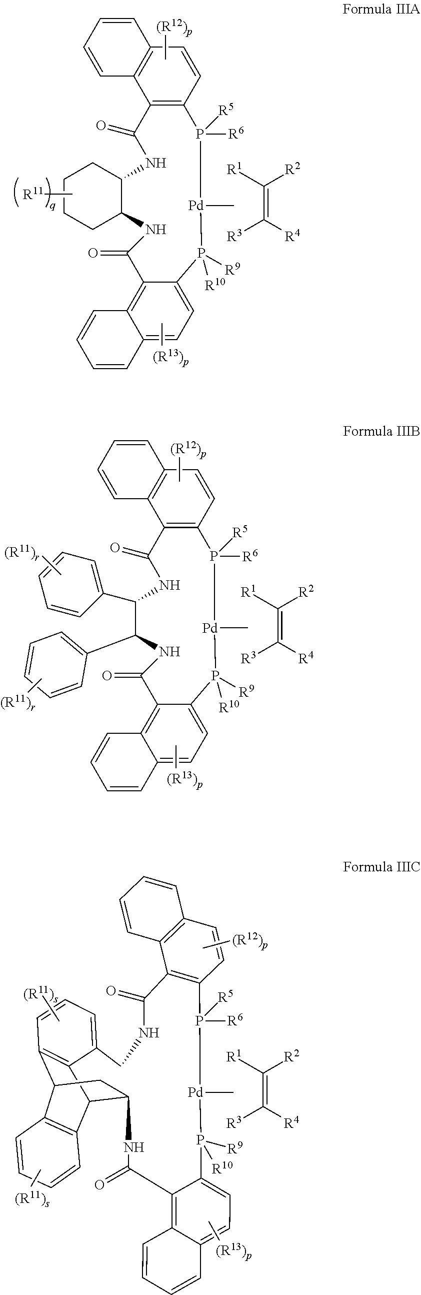

wherein:
each R¹¹ independently is selected from aliphatic, heteroaliphatic, haloaliphatic, aromatic, or an organic functional group;
each of R¹² and R¹³ independently, for each occurrence, is selected from aliphatic, heteroaliphatic, aromatic, haloaliphatic, or an organic functional group;
p is an integer selected from 0 to 6;
q is an integer ranging from 0 to 10;
each r independently is an integer selected from 0 to 5; and
each s independently is an integer selected from 0 to 4.

In any or all of the above embodiments, each R¹¹ independently is selected from alkyl, alkenyl, alkynyl, heteroalkyl, heteroalkenyl, heteroalkynyl, aryl, heteroaryl, nitro, hydroxyl, amine, halogen, cyano, thiol, and haloalkyl; and wherein each of R¹² and R¹³ independently, for each occurrence is selected from aliphatic, heteroaliphatic, aromatic, nitro, hydroxyl, amine, halogen, cyano, thiol, and haloalkyl.

In any or all embodiments, the chiral precatalyst has a structure according to Formula IV

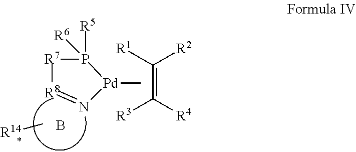

wherein:
R¹⁴ is selected from aliphatic, heteroaliphatic, aromatic, or an organic functional group; and
the * symbol indicates that R¹⁴ is chiral or is attached to an asymmetric carbon atom of ring B.

In any or all of the above embodiments, ring B is a five-membered or six-membered heterocycle.

In any or all of the above embodiments, R¹⁴ is an alkyl group that is attached to an asymmetric carbon atom of ring B.

In any or all of the above embodiments, the chiral precatalyst has a structure according to Formula IVA or IVB

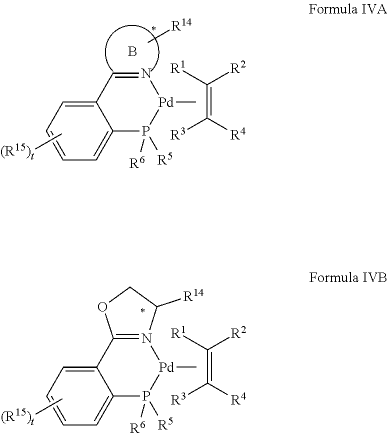

wherein:
each R¹⁵ independently is selected from aliphatic, heteroaliphatic, haloaliphatic, aromatic, or an organic functional group; and
t is an integer selected from 0 to 4.

In any or all of the above embodiments, each R¹⁵ independently is selected from alkyl, alkenyl, alkynyl, heteroalkyl, heteroalkenyl, heteroalkynyl, aryl, heteroaryl, nitro, hydroxyl, amine, halogen, cyano, thiol, and haloalkyl.

In any or all of the above embodiments, the chiral precatalyst is selected from a compound as recited in Table B herein.

Also disclosed herein is a method of making the chiral precatalyst according to any or all of the above chiral precatalyst embodiments, wherein the method comprises exposing a Pd(0) precursor complex having a structure according to Formula A to a donor atom-containing ligand compound having a structure according to Formula V:

wherein Formula A is

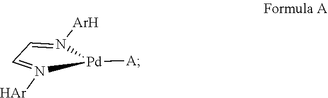

wherein A is as recited in claim 1 and Ar is aromatic; and

### Formula V is

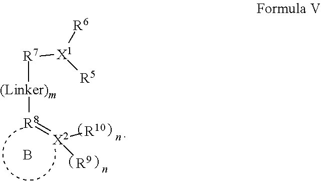

In any or all of the above method embodiments, the Pd(0) precursor complex is

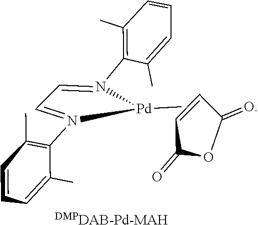

In any or all of the above embodiments, the donor atom-containing ligand compound has a structure according to Formula VI or Formula VII

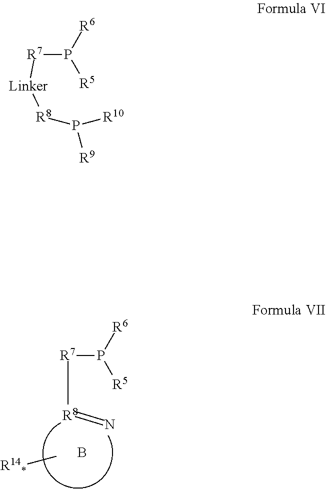

In any or all of the above embodiments, the donor atom-containing ligand compound has a structure according to Formula VIA, VIB, VIC, or VIIA

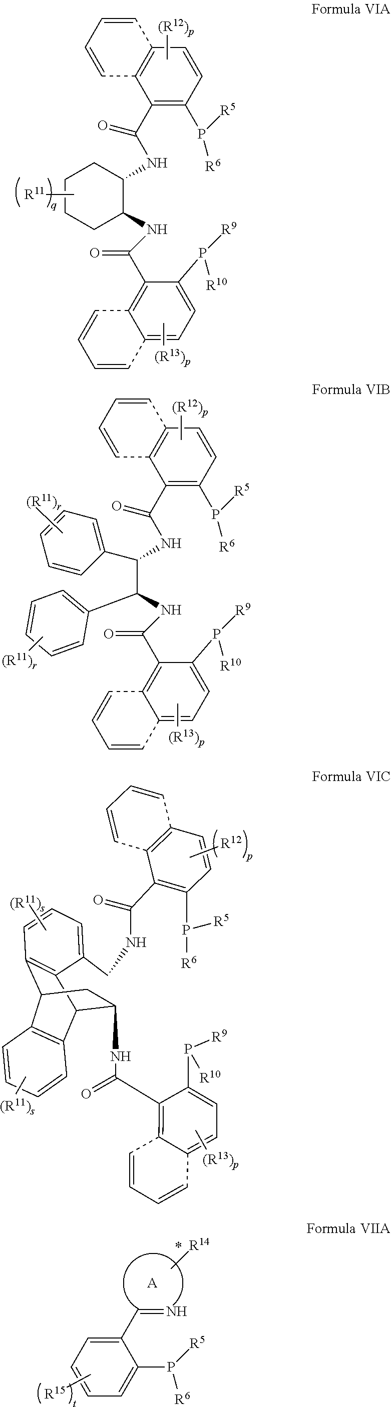

wherein:
each R¹¹ independently is selected from aliphatic, heteroaliphatic, haloaliphatic, aromatic, or an organic functional group;
each of R¹² and R¹³ independently, for each occurrence, is selected from aliphatic, heteroaliphatic, haloaliphatic, aromatic, or an organic functional group;
each R¹⁵ independently is selected from aliphatic, heteroaliphatic, haloaliphatic, aromatic, or an organic functional group;
p is an integer selected from 0 to 6;
q is an integer ranging from 0 to 10;
each r independently is an integer selected from 0 to 5;
each s independently is an integer selected from 0 to 4; and
t is an integer selected from 0 to 4.

Also disclosed herein are embodiments of a method, comprising using the chiral precatalyst according to any or all of the above embodiments as a catalyst in a palladium-mediated enantioselective chemical reaction.

In any or all of the above embodiments, the palladium-mediated enantioselective chemical reaction is an asymmetric allylation reaction.

In any or all of the above embodiments, the asymmetric allylation reaction is an asymmetric malonation reaction, a desymmetrization reaction, or an asymmetric ring-opening allylation reaction.

### VI. Examples

Materials. All solvents and common organic reagents were purchased from commercial suppliers and used without further purification. ^{DMP}DAB-Pd-MAH was prepared using the procedure we previously reported. (S,S)-^{NAPH}DACH ligand, (S,S)-^{Ph}DACH ligand, and (S)-iPr-PHOX ligand were purchased from Strem Chemicals and used as received. (S,S)-^{Ph}ANDEN ligand, (S,S)-^{Ph}STIL ligand, and (S)-tBuPHOX were prepared following reported procedures. Anhydrous solvents (SureSeal) were purchased from MilliporeSigma and used as received. As used herein, “rt”=room temperature; “equiv.”=equivalent; “equivs.”=equivalents; and “min”=minutes.

Techniques. Air-free manipulations in the preparation of catalysts were performed under a dry nitrogen atmosphere using an MBraun glovebox. Air free manipulations for catalytic evaluation were performed under a dry atmosphere using Nitrogen balloons.

Analysis and Spectroscopy. NMR spectra were acquired on a Bruker AVANCE 300 MHz spectrometer, a Bruker AVANCE 360 MHz, a Bruker AVANCE 400 MHz spectrometer, or a Bruker AVANCE Neo 500 MHz spectrometer. ¹H and 13C NMR chemical shifts are calibrated to residual protio-solvents and all ³¹P NMR chemical shifts are calibrated to external standards. NMR spectroscopic data is processed using Bruker TopSpin 4.07, or Mestrenova.

High-resolution electrospray ionization mass spectrometric analysis was performed using a Thermo Scientific Ultimate 3000 ESI-Orbitrap Exactive Plus.

High performance liquid chromatography analysis was carried out on an Agilent 1100 system, using a mixture of n-hexane and isopropyl alcohol with Chiralpak columns (250×4.6 mm; 5 μm)

### Example 1

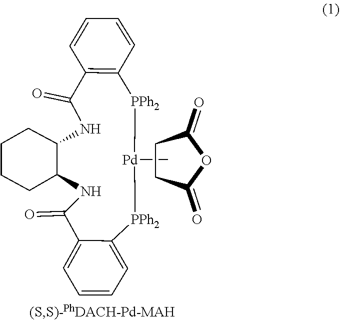

The metalation was done under an N₂ atmosphere following the procedure: In a 1-dram vial, (S,S)-^{Ph}DACH (11.1 mg, 0.0161 mmol) was dissolved in 0.2 mL THF-d8. To another 1-dram vial was added ^{DMP}DAB-Pd-MAH (6.3 mg, 0.0134 mmol), 1,3,5-trimethoxybenzene (2.3 mg, 0.0134 mmol), and 0.5 mL THF-d8. This palladium sample solution was transferred to an NMR tube, and a ¹H NMR spectrum (500 MHz; THF-d8) was obtained. (S,S)-^{Ph}DACH ligand solution was transferred to the NMR tube containing the palladium sample. Subsequent ¹H NMR and ³¹P NMR spectra were obtained after 5 minutes.

The entire synthetic procedure was performed in a glovebox under a dry, oxygen-free atmosphere of nitrogen gas. A 4-dram vial was charged with ^{DMP}DAB-Pd-MAH (100.1 mg, 0.21 mmol), (S,S)-^{Ph}DACH (147.2 mg, 0.21 mmol), and 4 mL of THF. The mixture was stirred at room temperature for one hour. THF was evaporated under vacuum followed by trituration/decantation with hexane/diethyl ether (1:1) until the washings were colorless (6 times). The product was dried and removed under vacuum to give a pale-yellow solid (121 mg, 63%).

¹H NMR: (500 MHz; THF-d₈) δ 1.12-1.24 (m, 2H, 2× cyclohexyl H), 1.24-1.33 (m, 1H, 1×cyclohexyl H), 1.53-1.65 (m, 2H, 2× cyclohexyl H), 1.79 (m, 1H, 1×cyclohexyl H), 2.05 (m, 1H, 1×cyclohexyl H), 2.21 (m, 1H, 1×cyclohexyl H), 3.37 (sept, J=5.14 Hz, 1H, NH—CH—CH—NH), 3.64 (m, 1H, NH—CH—CH—NH), 4.08 (m, 1H, MAH-H), 4.38 (m, 1H, MAH-H), 6.65-7.59 (m, 28H, Ar—H), 7.82 (d, J=6.31 Hz, 1H, NH), 8.06 (d, J=5.57 Hz, 1H, NH).

¹³C{¹H}NMR: (125 MHz; THF-d₈) δ 23.74 (cyclohexyl C), 24.82 (cyclohexyl C), 31.23 (cyclohexyl C), 32.06 (cyclohexyl C), 52.41 (CH—NH), 57.01 (C═C and 1× CH—NH), 126.91 (Ar), 127.00 (Ar), 127.29 (Ar), 127.37 (Ar), 127.81 (Ar), 127.88 (Ar), 128.04 (d, J_{CP}=Hz, Ar), 128.18 (d, J_{CP}=Hz), 128.66 (Ar), 128.69 (Ar), 128.73 (Ar), 129.00 (Ar), 129.04 (Ar), 129.28 (Ar), 129.32 (Ar), 129.40 (Ar), 129.47 (Ar), 129.54 (Ar), 129.61 (Ar), 132.98 (Ar), 132.47 (Ar), 133.10 (Ar), 133.93 (Ar), 134.21 (d, J_{CP}=2.62 Hz, Ar), 136.12 (Ar), 137.71 (Ar), 139.75 (Ar), 143.71 (Ar), 168.04 (d, ³J_{CP}=3.51 Hz, NH—C═O), 168.98 (MAH C═O), 170.26 (MAH C═O), 170.65 (d, ³J_{CP}=2.64 Hz, NH—C═O)

³¹P{¹H}NMR: (200 MHz; THF-d₈) δ 23.92 (d, ²J_{P-P}=4.85 Hz), 24.90 (d, ²J_{P-P}=4.88 Hz).

Two conformers were observed when 1 was dissolved in CD₂Cl₂.

¹H NMR: (500 MHz; CD₂Cl₂) δ 1.10-1.16 (m, 2H, cyclohexyl), 1.12-1.45 (m, 6H, cyclohexyl), 1.63-1.67 (m, 2H, cyclohexyl), 1.76-1.82 (m, 2H, cyclohexyl), 1.99-2.02 (m, 1H, cyclohexyl), 2.08-2.12 (m, 2H, cyclohexyl), 2.24-2.28 (m, 1H, cyclohexyl), 3.38 (septet, J=5.17 Hz, 1H, CH—NH, major conformer), 3.52-3.59 (m, 1H, CH—NH, minor conformer), 3.65-3.71 (m, 1H, CH—NH, major conformer), 3.80-3.87 (m, 1H, CH—NH, minor conformer), 3.92-3.99 (m, 2H, 1×MAH-H of major conformer, 1×MAH-H of minor conformer), 4.39-4.44 (m, 2H, 1×MAH-H of major conformer, 1×MAH-H of minor conformer), 6.14 (d, J=6.06 Hz, 1H, NH, major conformer), 6.65-7.60 (m, 56H, Ar—H), 7.86 (d, J=5.14 Hz, 1H, NH, major conformer).

³¹P{¹H}NMR: (200 MHz; CD₂Cl₂) δ 22.13 (d, ²J_{P-P}=11.94 Hz, minor conformer), 23.02 (d, ²J_{P-P}=7.13 Hz, major conformer), 24.54 (d, ²J_{P-P}=7.14 Hz, major conformer), 26.46 (d, ²J_{P-P}=12.01 Hz, minor conformer).

HRMS (ESI) of [C₄₈H₄₂N₂O₅P₂Pd·Na](major isotopomer, sodium adduct): 917.14960 (calc'd); 917.15105 (found).

### Example 2

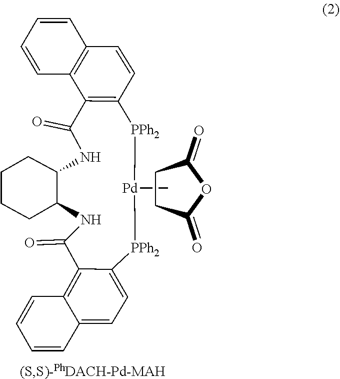

The metalation was done under an N₂ atmosphere following the procedure: In a 1-dram vial, (S,S)-^{NAPH}DACH (12.8 mg, 0.0161 mmol) was dissolved in 0.2 mL THF-d8. Another 1-dram vial was added ^{DMP}DAB-Pd-MAH (6.3 mg, 0.0134 mmol), 1,3,5-trimethoxybenzene (2.3 mg, 0.0134 mmol), and 0.5 mL THF-d8. This palladium sample solution was transferred to an NMR tube, and a ¹H NMR spectrum (500 MHz; THF-d8) was obtained. (S,S)-^{NAPH}DACH solution was transferred to the NMR tube containing the palladium sample. Subsequent ¹H NMR and ³¹P NMR spectra were obtained after 5 minutes.

The entire procedure was performed in a glovebox under a dry, oxygen-free atmosphere of nitrogen gas. A 4-dram vial was charged with of ^{DMP}DAB-Pd-MAH (100.1 mg, 0.21 mmol), (S,S)-^{NAPH}DACH (168.7 mg, 0.21 mmol), and 5 mL of THF. The solution was stirred for one hour. THE was evaporated under vacuum followed by trituration/decantation 6 times with hexane/diethyl ether (1:1) until the washings were colorless. Residual solvents from the trituration/decantation were removed under vacuum, and the solid was dried under vacuum overnight to give a pale-yellow powder (169.6 mg, 80%). The product was recrystallized from pentane/THF.

NMR signals of 2 were generally smaller than 1, due to the rapid conformer exchange. Some peaks are assigned as the analogue of 1.

¹H NMR: (500 MHz; THF-d₈) δ 1.28-1.57 (m, 4H, 4× cyclohexyl H), 1.75-1.84 (m, 2H, 2× cyclohexyl H), 2.18 (br s, 1H, 1×cyclohexyl H), 2.92 (br s, 1H, 1×cyclohexyl H), 3.57 (s, 1H, 1×NH—CH—CH—NH), 3.95 (sept, 1H, 1×NH—CH—CH—NH), 4.39 (s, 1H, 1×MAH-H), 4.57 (br s, 1H, 1×MAH-H), 6.76-7.91 (m, 32H, Ar—H and NH), 8.22 (br s, 2H, Ar—H).

¹³C{¹H}NMR: (125 MHz; THF-d₈) δ 24.87 (cyclohexyl C), 25.82 (cyclohexyl C), 32.24 (cyclohexyl C), 32.65 (cyclohexyl C), 54.14 (CH—NH), 54.28 (CH—NH), 57.04 (C═C), 59.36 (br, C=C), 126.45 (Ar), 126.68 (Ar), 127.67 (Ar), 127.81 (Ar), 127.89 (Ar), 128.10 (Ar), 128.19 (d, J=2.61 Hz, Ar), 128.28 (Ar), 128.78 (Ar), 128.84 (Ar), 128.99 (Ar), 129.08 (Ar), 129.56 (Ar), 129.65 (d, J=4.41 Hz, Ar), 130.00 (d, J=1.75 Hz, Ar), 130.31 (Ar), 130.60 (Ar), 132.07 (Ar), 132.13 (Ar), 132.20 (Ar), 132.28 (Ar), 133.43 (d, J=3.52 Hz, Ar), 133.62 (d, J=4.34 Hz, Ar), 134.52 (Ar), 134.63 (Ar), 134.83 (Ar), 134.94 (Ar), 134.99 (Ar), 135.06 (Ar), 135.24 (Ar), 135.45 (Ar), 135.69 (Ar), 136.03 (Ar), 136.29 (Ar), 136.50 (Ar), 136.62 (Ar), 168.01 (C═O), 170.58 (br, C═O), 170.96 (C═O).

³¹P{¹H}NMR: (200 MHz; THF-d₈) δ 22.82, 23.17 (br).

HRMS (ESI) of [C₅₆H₄₆N₂₀₅P₂Pd·Na](major isotopomer, sodium adduct): 1017.18090 (calc'd); 1017.18275 (found).

Single crystals of C₆₀H₅₄N₂O₆P₂Pd (2·THF, precatalyst 2 with one THE molecule co-crystallized) were selected using a MitEGen loop and paratone oil. A suitable crystal was selected and run on a Bruker APEX-II CCD diffractometer. The crystal was kept at 100.15 K during data collection. Using Olex2, the structure was solved with the olex2.solve structure solution program using Charge Flipping and refined with the XL refinement package using Least Squares minimisation.

Crystal structure determination of (2·THF)

Crystal Data for C₆₀H₅₄N₂O₆P₂Pd (M=1067.39 g/mol): orthorhombic, space group P2₁2₁2₁ (no. 19), a=13.443(10) Å, b=16.517(11) Å, c=23.465(17) Å, V=5210(6) Å³, Z=4, T=100.15 K, p(MoKa)=0.471 mm⁻¹, Dcalc=1.361 g/cm³, 83698 reflections measured (3.016°<20 5 54.84°), 11652 unique (Rᵢₙₜ=0.0456, R_{sigma}=0.0325) which were used in all calculations. The final R¹ was 0.0252 (I>2σ-(I)) and wR₂ was 0.0569 (all data).

TABLE 1
Crystal data and structure refinement for 2•THF.

| Identification code | 2•THF | |
|---|---|---|
| Empirical formula | C₆₀H₅₄N₂O₆P₂Pd | |
| Formula weight | 1067.39 | |
| Temperature/K | 100.15 | |
| Crystal system | orthorhombic | |
| Space group | P2₁2₁2₁ | |
| a/Å | 13.443 | (10) |
| b/Å | 16.517 | (11) |
| c/Å | 23.465 | (17) |
| α/° | 90 | |
| β/° | 90 | |
| γ/° | 90 | |
| Volume/Å³ | 5210 | (6) |
| Z | 4 | |
| ρ_{calc}g/cm³ | 1.361 | |
| μ/mm⁻¹ | 0.471 | |
| F(000) | 2208.0 | |
| Crystal size/mm³ | 0.05 × 0.03 × 0.02 | |
| Radiation | MoKα (λ = 0.71073) | |
| 2Θ range for data collection/° | 3.016 to 54.84 | |
| Index ranges | −17 ≤ h ≤ 17, −19 ≤ k ≤ | |
| | 21, −30 ≤ \| ≤ 30 | |
| Reflections collected | 83698 | |
| Independent reflections | 11652 [Rᵢₙₜ = 0.0456, R_{sigma} = 0.0325] | |
| Data/restraints/parameters | 11652/0/641 | |
| Goodness-of-fit on F² | 1.025 | |
| Final R indexes [I >= 2σ (I)] | R₁ = 0.0252, wR₂ = 0.0545 | |
| Final R indexes [all data] | R₁ = 0.0322, wR₂ = 0.0569 | |
| Largest diff. peak/hole/e Å⁻³ | 0.23/−0.26 | |
| Flack parameter | −0.021 | (16) |

### Example 3

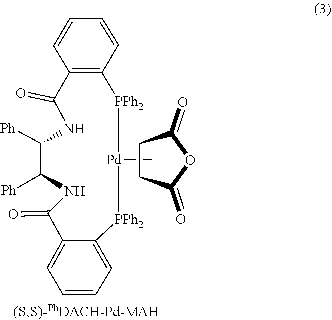

The metalation was done under an N₂ atmosphere following the procedure: In a 1-dram vial, (S,S)-^{Ph}STIL ((+)-1(S),2(S)-Bis(2′-(diphenylphosphino)benzamido]-1,2-diphenylethane) (11.1 mg, 0.0148 mmol) was dissolved in 0.2 mL THF-d8. Another 1-dram vial was added ^{DMP}DAB-Pd-MAH (6.3 mg, 0.0134 mmol), 1,3,5-trimethoxybenzene (2.3 mg, 0.0134 mmol), and 0.5 mL THF-d8. This palladium sample solution was transferred to an NMR tube, and a ¹H NMR spectrum (500 MHz; THF-d8) was obtained. (S,S)-^{Ph}STIL ligand solution was transferred to the NMR tube containing the palladium sample. Subsequent ¹H NMR and ³¹P NMR spectra were obtained after 5 minutes.

The entire procedure was performed under an inert atmosphere. A 4-dram vial was charged with of ^{DMP}DAB-Pd-MAH (100.1 mg, 0.21 mmol), (S,S)-^{Ph}STIL (168.5 mg, 0.21 mmol) and 6 mL of THF. The reaction mixtures were stirred for three hours. The solution was filtered through a celite bed. The solvent was evaporated under vacuum, followed by trituration/decantation 6 times with hexanes until the washing was colorless. Residual solvents from the trituration/decantation were removed under vacuum, and the desired product was precipitated from 1 mL THF and 4 mL hexanes. The product was finally dried under vacuum to give a tan solid (146.2 mg, 68%).

Two conformers were observed when 3 was dissolved in THF.

¹H NMR: (500 MHz; THF-d₈) minor conformer b 4.08 (m, 1H, 1×MAH-H), 4.52 (m, 1H, 1×MAH-H), 5.11 (m, 1H, CH, overlapped with the major conformer), 5.55 (m, 1H, CH), 6.71-7.66 (m, 38H, Ar—H and 1×NH) 8.16 (d, 1H, N—H).

major conformer b 4.27 (m, 1H, 1×MAH-H), 4.60 (m, 1H, 1×MAH-H), 5.13 (m, 1H, CH, overlapped with the minor conformer), 5.28 (m, 1H, CH), 6.71-7.66 (m, 38H, Ar—H), 8.06 (d, 1H, N—H), 8.66 (d, 1H, N—H).

¹3C NMR: (125 MHz; THF-d₈) δ 53.76 (dd, ²J_{CP}=24.66 Hz, C=C, another coupling constant was not able to be determined due to low signal-to-noise ratio), 54.56 (dd, ²J_{CP}=26.42 Hz, C=C, another coupling constant was not able to be determined due to low signal-to-noise ratio), 57.15 (dd, ²J_{CP1}=3.51 Hz, ²J_{CP}2=24.72 Hz, C=C), 57.15 (dd, ²J_{CP}1=3.51 Hz, ²J_{CP}2=24.72 Hz, C=C), 57.67 (dd, ²J_{CP}1=4.41 Hz, ²J_{CP}2=24.72 Hz, C=C), 59.47 (CH), 59.91 (CH), 61.11 (CH), 61.97 (CH), 126.40 (Ar), 126.85 (Ar), 126.93 (Ar), 127.04 (Ar), 127.12 (Ar), 127.30 (Ar), 127.39 (Ar), 127.44 (Ar), 127.48 (Ar), 127.51 (Ar), 127.64 (Ar), 127.71 (Ar), 127.92 (Ar), 127.97 (Ar), 128.00 (Ar), 128.09 (Ar), 128.13 (Ar), 128.21 (Ar), 128.31 (Ar), 128.52 (Ar), 128.58 (Ar), 128.69 (Ar), 128.74 (Ar), 128.87 (Ar), 128.91 (Ar), 129.17 (Ar), 129.22 (Ar), 129.33 (d, J=2.65 Hz, Ar), 129.42 (Ar), 129.46 (Ar), 129.53 (Ar), 129.60 (Ar), 129.68 (Ar), 129.72 (Ar), 130.76 (d, J=1.75 Hz, Ar), 132.56 (Ar), 133.10 (Ar), 133.21 (Ar), 133.48 (Ar), 133.59 (Ar), 133.73 (Ar), 133.78 (Ar), 133.89 (Ar), 134.03 (Ar), 134.17 (Ar), 134.41 (Ar), 134.58 (Ar), 134.79 (Ar), 134.83 (Ar), 134.98 (Ar), 135.01 (Ar), 135.04 (Ar), 135.08 (Ar), 135.14 (Ar), 135.20 (Ar), 135.26 (Ar), 135.54 (Ar), 135.58 (Ar), 135.80 (Ar), 135.83 (Ar), 135.96 (Ar), 136.10 (Ar), 136.23 (Ar), 137.32 (Ar), 137.35 (Ar), 137.63 (Ar), 137.68 (Ar), 137.80 (Ar), 139.03 (Ar), 139.12 (Ar), 139.17 (Ar), 141.20 (Ar), 141.45 (Ar), 143.14 (Ar), 143.30 (Ar), 167.79 (d, ³J_{CP}=3.54 Hz, NH—CO), 169.05 (d, ³J_{CP}=4.43 Hz, MAH CO), 170.22 (d, ³J_{CP}=3.46 Hz, MAH CO), 170.77 (d, 3J_{CP}=2.66 Hz, NH—CO).

³¹P{¹H}NMR: (200 MHz; THF-d₈) δ 23.27 (d, ²J_{PP}=10.06 Hz, minor conformer), 24.01 (d, ²J_{PP}=5.32 Hz, major conformer), 24.61 (d, ²J_{PP}=5.37 Hz, major conformer), 26.72 (d, ²J_{PP}=9.94 Hz, minor conformer).

HRMS (ESI) of [C₅₆H₄₄N₂O₅P₂Pd·Na](major isotopomer, sodium adduct): 1015.16525 (calc'd); 1015.16711 (found).

### Example 4

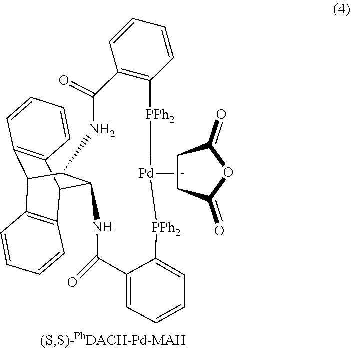

The metalation was done under an N₂ atmosphere following the procedure: In a 1-dram vial, (S,S)-^{Ph}ANDEN (13.1 mg, 0.0161 mmol) was dissolved in 0.2 mL THF-d8. Another 1-dram vial was added ^{DMP}DAB-Pd-MAH (6.3 mg, 0.0134 mmol), 1,3,5-trimethoxybenzene (2.3 mg, 0.0134 mmol), and 0.5 mL THF-d8. This palladium sample solution was transferred to an NMR tube, and a ¹H NMR spectrum (500 MHz; THF-d8) was obtained. (S,S)-^{Ph}ANDEN ligand solution was transferred to the NMR tube containing the palladium sample. Subsequent ¹H NMR and ³¹P NMR spectra were obtained after 5 minutes.

The entire procedure was performed under an inert atmosphere. A 4-dram vial was charged with ^{DMP}DAB-Pd-MAH (84.0 mg, 0.18 mmol), (S,S)-^{Ph}ANDEN (160.2 mg, 0.20 mmol), and 5 mL of THF. The solution was stirred for one hour. THF was evaporated under vacuum followed by trituration/decantation with hexane/diethyl ether (1:1) (12 times) until the washings were colorless. The product was dried under vacuum to give a yellow solid (116.0 mg, 50%). The product was recrystallized from TBME/THF.

¹H NMR: (500 MHz; THF-d₈) δ 3.04 (m, 1H, 1×MAH-H), 3.70 (m, 1H, CH—NH), 3.80 (m, 1H, CH—NH), 4.28 (dd, 2H, 2× CH), 4.48 (m, 1H, 1×MAH-H), 6.70-7.48 (m, 36H, Ar—H&NH), 7.57 (dd, J₁=7.36 Hz, J₂=1.87 Hz, 1H, Ar—H), 7.80 (dd, J_{1=7.55} Hz, J₂=2.52 Hz, 1H, Ar—H).

¹³C{¹H}NMR: (125 MHz; THF-d₈) δ 48.37 (CH-Ph), 50.01 ((CH-Ph), 56.64 (C=C), 56.66 (C=C), 57.00 (CH—NH), 59.96 (CH—NH), 59.96 (CH), 123.25 (Ar), 124.02 (Ar), 125.58 (Ar), 125.71 (Ar), 125.83 (Ar), 125.90 (Ar), 125.99 (Ar), 126.37 (Ar), 127.08 (Ar), 127.16 (Ar), 127.32 (Ar), 127.40 (Ar), 127.47 (Ar), 127.79 (Ar), 127.87 (d, J=3.51 Hz, Ar), 127.96 (d, J=3.49 Hz, Ar), 128.17 (Ar), 128.25 (Ar), 128.31 (Ar), 128.49 (Ar), 129.08 (d, J=3.51 Hz, Ar), 129.58 (Ar), 129.69 (d, J=5.29 Hz, Ar), 132.11 (Ar), 132.23 (Ar), 133.02 (Ar), 133.14 (Ar), 133.24 (Ar), 134.42 (Ar), 135.55 (Ar), 135.66 (Ar), 136.31 (Ar), 136.33 (d, J=3.51 Hz, Ar), 136.51 (Ar), 136.56 (Ar), 136.66 (Ar), 138.44 (Ar), 139.98 (Ar), 142.69 (Ar), 142.95 (Ar), 168.31 (d, ³J_{CP}=3.52 Hz, NHC═O), 168.97 (MAH C═O), 170.25 (d, ³J_{CP}=3.53 Hz, MAH C═O).

³¹P{¹H}NMR: (200 MHz, THF-d₈) 627.61.

HRMS (ESI) of [C₅₈H₄₄N₂O₅P₂Pd·Na](major isotopomer, sodium adduct): 1039.16525 (calc'd); 1039.16723 (found).

Single crystals of C₆₆H₅₈N₂O₇P₂Pd (4.2THF, precatalyst 4 with two THF molecules co-crystallized) were selected using a MitEGen loop and paratone oil. A suitable crystal was selected and run on a Bruker APEX-II CCD diffractometer. The crystal was kept at 100 K during data collection. Using Olex2, the structure was solved with the olex2.solve structure solution program using Charge Flipping and refined with the XL refinement package using Least Squares minimization. Squeeze was used on four molecules of tetrahydrofuran, used as solvent, due to it being highly disordered.

### Crystal Structure Determination of (4.2THF)

Crystal Data for C₆₆H₅₈N₂O₇P₂Pd (M=1159.48 g/mol): orthorhombic, space group P2₁2₁2₁ (no. 19), a=14.552(3) Å, b=16.681(3) Å, c=26.014(5) Å, V=6314(2) Å³, Z=57, T=100 K, p(MoKa)=0.395 mm⁻¹, Dcalc=1.220 g/cm³, 78493 reflections measured (2.9°≤2Θ≤52.604°), 12630 unique (Rᵢₙₜ=0.0988, R_{sigma}=0.0715) which were used in all calculations. The final R₁ was 0.0546 (1>2σ(1)) and wR₂ was 0.1439 (all data).

TABLE 2
Crystal data and structure refinement for 4•2THF.

| Identification code | 4•2THF | |
|---|---|---|
| Empirical formula | C₆₆H₅₈N₂O₇P₂Pd | |
| Formula weight | 1159.48 | |
| Temperature/K | 100 | |
| Crystal system | orthorhombic | |
| Space group | P2₁2₁2₁ | |
| a/Å | 14.552 | (3) |
| b/Å | 16.681 | (3) |
| c/Å | 26.014 | (5) |
| α/° | 90 | |
| β/° | 90 | |
| γ/° | 90 | |
| Volume/Å³ | 6314 | (2) |
| Z | 57 | |
| ρ_{calc}g/cm³ | 1.220 | |
| μ/mm⁻¹ | 0.395 | |
| F(000) | 2400.0 | |
| Crystal size/mm³ | 0.0176 × 0.0132 × 0.0078 | |
| Radiation | MoKα (λ = 0.71073) | |
| 2Θ range for data collection/° | 2.9 to 52.604 | |
| Index ranges | −18 ≤ h ≤ 18, −20 ≤ k ≤ | |
| | 20, −31 ≤ \| ≤ 32 | |
| Reflections collected | 78493 | |
| Independent reflections | 12630 [Rᵢₙₜ = 0.0988, R_{sigma} = 0.0715] | |
| Data/restraints/parameters | 12630/0/703 | |
| Goodness-of-fit on F² | 1.027 | |
| Final R indexes [I >= 2σ (I)] | R₁ = 0.0546, wR₂ = 0.1325 | |
| Final R indexes [all data] | R₁ = 0.0740, wR₂ = 0.1439 | |
| Largest diff. peak/hole/e Å⁻³ | 1.38/−1.93 | |
| Flack parameter | −0.017 | (13) |

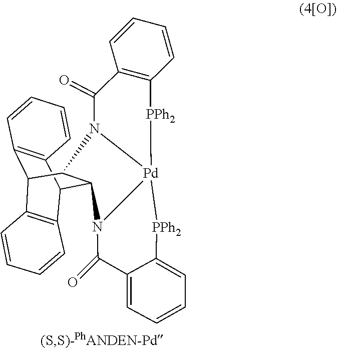

Single crystals of C₅₉H₅₂N₂O₃P₂Pd (4[O]·TBME, precatalyst 4[O] plus one molecule of TBME co-crystallized) were selected using a MitEGen loop and paratone oil. A suitable crystal was selected and run on a Bruker APEX-II CCD diffractometer. The crystal was kept at 99.93 K during data collection. Using Olex2, the structure was solved with the olex2.solve structure solution program using Charge Flipping and refined with the XL refinement package using Least Squares minimization.

### Crystal Structure Determination of 4[O]·TBME

Crystal Data for C₅₉H₅₂N₂O₃P₂Pd (M=1005.42 g/mol): monoclinic, space group P₂₁/n (no. 14), a=15.203(11) Å, b=21.082(15) Å, c=16.227(12) Å, 6=112.831(15°), V=4793(6) Å³, Z=43, T=99.93 K, p(MoKa)=0.503 mm⁻¹, Dcalc=1.393 g/cm³, 38884 reflections measured (3.118°≤2Θ≤52.68°), 9436 unique (Rᵢₙₜ=0.1010, R_{sigma}=0.1003) which were used in all calculations. The final R¹ was 0.0511 (I>2σ-(I)) and wR₂ was 0.1192 (all data).

TABLE 3
Crystal data and structure refinement for (4[O]•TBME).

| Identification code | 3[O] | |
|---|---|---|
| Empirical formula | C₅₉H₅₂N₂O₃P₂Pd | |
| Formula weight | 1005.42 | |
| Temperature/K | 99.93 | |
| Crystal system | monoclinic | |
| Space group | P2₁/n | |
| a/Å | 15.203 | (11) |
| b/Å | 21.082 | (15) |
| c/Å | 16.227 | (12) |
| α/° | 90 | |
| β/ | 112.831 | (15) |
| γ/° | 90 | |
| Volume/Å³ | 4793 | (6) |

| Z | 43 |
|---|---|
| ρ_{calc}g/cm³ | 1.393 |
| μ/mm⁻¹ | 0.503 |
| F(000) | 2080.0 |
| Crystal size/mm³ | 0.01 × 0.008 × 0.007 |
| Radiation | MoKα (λ = 0.71073) |
| 2Θ range for data collection/° | 3.118 to 52.68 |
| Index ranges | −18 ≤ h ≤ 18, −23 ≤ k ≤ |
| | 25, −20 ≤ \| ≤ 20 |
| Reflections collected | 38884 |
| Independent reflections | 9436 [Rᵢₙₜ = 0.1010, R_{sigma} = 0.1003] |
| Data/restraints/parameters | 9436/0/608 |
| Goodness-of-fit on F² | 1.017 |
| Final R indexes [I >= 2σ (I)] | R₁ = 0.0511, wR₂ = 0.1045 |
| Final R indexes [all data] | R₁ = 0.0940, wR₂ = 0.1192 |
| Largest diff. peak/hole/e Å⁻³ | 1.24/−0.86 |

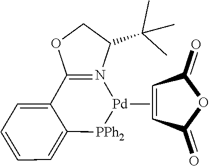

The metalation was done under an N₂ atmosphere by following the procedure: In a 1-dram vial, (S)-^{tBu}PHOX ligand (6.2 mg, 0.0161 mmol) was dissolved in 0.3 mL THF-d8. Another 1-dram vial was added ^{DMP}DAB-Pd-MAH (5.8 mg, 0.0124 mmol), 1,3,5-trimethoxybenzene (2.1 mg, 0.0124 mmol), and 0.4 mL THF-d8. This palladium sample solution was transferred to an NMR tube, and a ¹H NMR spectrum (500 MHz; THF-d8) was obtained. (S)-^{Bu}PHOX ligand solution was transferred to the NMR tube containing palladium sample. Subsequent ¹H NMR and ³¹P NMR spectra were obtained after 5 minutes.

The entire procedure was performed on the bench. A 4-dram vial was charged with of ^{DMP}DAB-Pd-MAH (167.4 mg, 0.36 mmol), (S)-^{tBu}PHOX ligand (140 mg, 0.36 mmol), and 8 mL of THF. The solution was stirred for 3 hours. The reaction mixtures were filtered through a celite bed followed by the solvent evaporation. The crude product was then triturated/decanted 8× with hexane until the washing was colorless. Remaining solvents from the trituration/decantation was removed under vacuum and dried overnight to a brown solid (191.9 mg, 90%).

¹H NMR: (500 MHz; CD₂Cl₂): minor conformer δ 0.62 (s, 3H, CH₃), 3.78-3.81 (m, 1H, 1×MAH-H), 4.07 (dd, J₁=9.28 Hz, J₂=4.28, 1H, CH), 4.12-4.17 (m, 1H, 1×MAH-H), 4.36-4.44 (m, 2H, CH₂), 6.92-6.98 (m, 1H, Ar—H), 7.17-7.56 (m, 12H, Ar—H), 8.15-8.18 (m, 1H, Ar—H)

major conformer δ 0.66 (s, 3H, CH₃), 3.78-3.81 (m, 1H, 1×MAH-H), 4.12-4.17 (m, 1H, CH), 4.23 (t, 1H, 1×MAH-H), 4.36-4.44 (m, 2H, CH₂), 6.92-6.98 (m, 1H, Ar—H), 7.17-7.56 (m, 12H, Ar—H), 8.15-8.18 (m, 1H, Ar—H)

¹³C NMR (125 MHz; CD₂Cl₂): δ 24.5 (CH₃), 25.27 (CH₃), 34.39 (C(CH₃)₃), 45.73 (C=C), 46.69 (d, ²J_{CP}=33.49 Hz, C=C), 47.20 (C=C), 48.54 (d, ²J_{CP}=32.62 Hz, C=C), 68.83 (CH₂), 68.91 (CH₂), 79.94 (d, 3J_{CP}=1.72 Hz, CH), 80.22 (d, ³J_{CP}=1.77 Hz, CH), 128.74 (Ar), 128.82 (Ar), 128.87 (d, J=2.65 Hz, Ar), 128.96 (m, Ar), 129.06 (Ar), 130.41 (Ar), 130.49 (d, J=2.61 Hz, Ar), 130.52 (Ar), 130.57 (d, J=1.77 Hz, Ar), 130.80 (d, J=1.76 Hz, Ar), 130.86 (d, J=1.76 Hz, Ar), 132,37 (Ar), 132.40 (Ar), 132.43 (Ar), 132.48 (d, J=3.52 Hz, Ar), 132.56 (d, J=1.77 Hz, Ar), 132.63 (Ar), 132.68 (Ar), 133.47 (Ar), 133.59 (Ar), 133.71 (Ar), 133.83 (Ar), 134.14 (Ar), 134.27 (Ar), 134.42 (Ar), 134.55 (Ar), 134.61 (Ar), 163.99 (O—C═N), 164.55 (O—C═N), 171.44 (d, ³J_{CP}=5.33 Hz, C═O), 171.81 (d, ³J_{CP}=5.29 Hz, C═O), 172.93 (C═O), 172.99 (C═O)

³¹P{¹H}NMR: (200 MHz; CD₂Cl₂) δ 22.29 (major conformer), 23.12 (minor conformer)

HRMS (ESI) of [C₂₉H₂₉NO₄PPd·H](major isotopomer, proton adduct): 592.08635 (calc'd); 592.08628 (found).

### Example 6

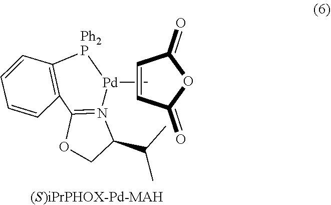

The metalation was done under an N₂ atmosphere by following the procedure: In a 1-dram vial, (S)-^{tPr}PHOX ligand (5.8 mg, 0.0155 mmol) was dissolved in 0.2 mL THF-d8. Another 1-dram vial was added ^{DMP}DAB-Pd-MAH (6.3 mg, 0.0134 mmol), 1,3,5-trimethoxybenzene (2.3 mg, 0.0134 mmol), and 0.5 mL THF-d8. This palladium sample solution was transferred to an NMR tube, and a ¹H NMR spectrum (500 MHz; THF-d8) was obtained. (S)-^{tPr}PHOX ligand solution was transferred to the NMR tube containing palladium sample. Subsequent ¹H NMR and ³¹P NMR spectra were obtained after 5 minutes.

The entire synthetic procedure was performed under an inert atmosphere. A 4-dram vial was charged with of ^{DMP}DAB-Pd-MAH (125.0 mg, 0.27 mmol), (S)-^{tPr}PHOX ligand (99.6 mg, 0.27 mmol), and 5 mL of THF. The solution was stirred for one hour. The reaction mixtures were filtered through a celite bed, followed by the evaporation of THF. The crude product was then triturated/decanted 8× with hexane/diethyl ether (1:1) until the washings were colorless. Residual solvents from the trituration/decantation was removed under vacuum and dried overnight to give a light green solid (112.8 mg, 73%).

The product was recrystallized from Pentane/THF.

Two conformers were observed in DCM.

¹H NMR: (500 MHz; CD₂Cl₂): minor conformer δ 0.21 (d, J=6.84 Hz, 3H, (CH₃)₂), 0.83 (d, J=6.84 Hz, 3H, (CH₃)₂), 2.09 (m, 1H, CH(CH₃)₂), 3.82 (m, 1H, 1×MAH-H), 4.11 (m, 1H, 1×MAH-H), 4.27-4.40 (m, 3H, CHCH₂), 7.01-7.05 (m, 1H, Ar—H), 7.23-7.56 (m, 12H, Ar—H), 8.08-8.13 (m, 1H, Ar—H).

major conformer δ 0.38 (d, J=6.83 Hz, 3H, (CH₃)₂), 0.87 (m, 3H, (CH₃)₂), 2.31 (m, 1H, CH(CH₃)₂), 3.91 (m, 1H, 1×MAH-H), 4.15 (m, 1H, 1×MAH-H), 4.27-4.40 (m, 3H, CHCH₂), 7.01-7.05 (m, 1H, Ar—H), 7.23-7.56 (m, 12H, Ar—H), 8.08-8.13 (m, 1H, Ar—H).

³¹P{¹H}NMR: (200 MHz; CD₂Cl₂) δ 21.68 (major conformer), 22.29 (minor conformer)

¹³C NMR: (125 MHz; CD₂Cl₂): major δ 13.25 (CH₃), 13.86 (CH₃), 18.37 (CH₃), 30.84 (CH(CH₃)₂), 31.00 (CH(CH₃)₂), 46.02 (d, ²J_{CP}=5.35 Hz, C=C), 46.26 (C=C), 46.82 (d, ²J_{CP}=4.29 Hz, C=C), 47.06 (C=C), 67.75 (CH₂), 67.76 (CH₂), 75.53, (CHCH₂), 75.58 (d, ⁴J_{CP}=1.80 Hz, CHCH₂), 128.80 (Ar), 128.85 (Ar), 128.87 (d, J_{CP}=1.81 Hz, Ar), 128.93 (d, J_{CP}=3.56 Hz, Ar), 129.02 (Ar), 129.10 (Ar) 130.49 (Ar), 130.53 (d, J_{CP}=1.77 Hz, Ar), 130.58 ((d, J_{CP}=1.80 Hz, Ar), 130.69 (d, J_{CP}=1.70 Hz, Ar), 130.80 (d, J_{CP}=1.73 Hz, Ar) 132.01 (Ar), 132.04 (Ar), 132.06 (Ar), 132.09 (Ar), 132.20 (Ar), 132.22 (Ar), 132.23 (Ar), 132.25 (Ar), 133.49 (Ar), 133.61 (Ar), 133.75 (Ar), 133.88 (Ar), 133.99 (d, J_{CP}=3.54 Hz, Ar), 134.12 (d, J_{CP}=5.32 Hz, Ar), 134.46 (Ar), 163.33 (O—C═N), 163.71 (O—C═N), 171.43 (d, ³J_{CP}=6.17 Hz, C═O), 171.88 (d, ³J_{CP}=5.32 Hz, C═O), 172.61 (C═O), 172.76 (C═O).

HRMS (ESI) of [C₂₈H₂₆NO₄PPd·Na](major isotopomer, Na⁺ adduct): 600.05265 (calc'd); 600.05253 (found).

Single crystals of C₂₈H₂₆NO₄PPd (6) were selected using a MitEGen loop and paratone oil. A suitable crystal was selected and run on a Bruker APEX-II CCD diffractometer. The crystal was kept at 100.02 K during data collection. Using Olex2, the structure was solved with the olex2.solve structure solution program using Charge Flipping and refined with the XL refinement package using Least Squares minimization. Squeeze was used on a solvent accessible void containing hexanes due to it being highly disordered.

### Crystal Structure Determination of 6

Crystal Data for C₂₈H₂₆NO₄PPd (M=577.87 g/mol): monoclinic, space group 12 (no. 5), a=14.747(9) Å, b=10.539(6) Å, c=18.153(16) Å, #=97.830(8°), V=2795(3) Å³, Z=24, T=100.02 K, p(MoKa)=0.752 mm⁻¹, Dcalc=1.373 g/cm³, 13250 reflections measured (3.344°≤2Θ≤52.58°), 4943 unique (Rᵢₙₜ=0.0807, R_{sigma}=0.1023) which were used in all calculations. The final R¹ was 0.0789 (I>2σ-(I)) and wR₂ was 0.2078 (all data).

TABLE 4
Crystal data and structure refinement for 6.

| Identification code | 6 | |
|---|---|---|
| Empirical formula | C₂₈H₂₆NO₄PPd | |
| Formula weight | 577.87 | |
| Temperature/K | 100.02 | |
| Crystal system | monoclinic | |
| Space group | I2 | |
| a/Å | 14.747 | (9) |
| b/Å | 10.539 | (6) |
| c/Å | 18.153 | (16) |
| α/° | 90 | |
| β/° | 97.830 | (8) |
| γ/° | 90 | |
| Volume/Å³ | 2795 | (3) |
| Z | 24 | |
| ρ_{calc}g/cm³ | 1.373 | |
| μ/mm⁻¹ | 0.752 | |
| F(000) | 1176.0 | |
| Crystal size/mm³ | 0.019 × 0.006 × 0.005 | |
| Radiation | MoKα (λ = 0.71073) | |
| 2Θ range for data collection/° | 3.344 to 52.58 | |
| Index ranges | −18 ≤ h ≤ 18, −13 ≤ k ≤ | |
| | 11, −22 ≤ \| ≤ 22 | |
| Reflections collected | 13250 | |
| Independent reflections | 4943 [Rᵢₙₜ = 0.0807, R_{sigma} = 0.1023] | |
| Data/restraints/parameters | 4943/104/318 | |
| Goodness-of-fit on F² | 1.017 | |
| Final R indexes [I >= 2σ (I)] | R₁ = 0.0789, wR₂ = 0.1879 | |
| Final R indexes [all data] | R₁ = 0.1098, wR₂ = 0.2078 | |
| Largest diff. peak/hole/e Å⁻³ | 3.72/−1.11 | |
| Flack parameter | 0.05 | (4) |

With reference to Examples 1-6, reaction monitoring by ³¹P NMR spectroscopy revealed complete consumption of the free phosphines donor atom-containing ligands within less than 20 minutes at room temperature in THF; this rapid ligand substitution is also evident by a near-immediate color change from red/purple to yellow. On preparative-scale, these complexes can be easily purified by simple precipitation to remove the soluble ^{DMP}DAB, giving the chiral Pd(0) complexes in 50-90% yield.

### Example 7

### (+)-1(S),2(S)-Bis(2′-(diphenylphosphino)benzamido]-1,2-diphenylethane ((S,S)-^{Ph}STIL)

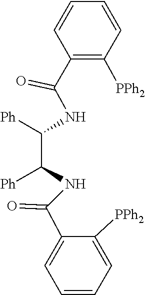

A 100 ml RB flask was added 2-(diphenylphosphanyl)benzoic acid (3809.3 mg, 12.44 mmol), 4-dimethylaminopyridine (68.3 mg, 0.56 mmol), EDAC (2104.2 mg, 13.55 mmol), and 42 mL DCM, followed by the final addition of (1 S,2S)-(−)-1,2-diphenylethylenediamine (1202.4 mg, 5.66 mmol). The mixtures were stirred at room temperature overnight. The reaction solution was diluted with 80 mL diethyl ether, and the organic phase was washed with 10% HCl (3×80 mL), water (1×80 mL), saturated aqueous NaHCO₃ (2×80 mL), 1 M NaOH (1×80 mL) and brine (1×80 mL), followed by the addition of anhydrous MgSO₄. The solvents were evaporated to give the crude product. The crude product was further purified by flash chromatography (ethyl acetate/hexanes) to yield a white solid. The white solid was redissolved in THF and dried with CaH₂ in N₂ atmosphere. The desire product was obtained after solvent evaporation under vacuum (895 mg, 20%).

To a solution of 2-(diphenylphosphanyl)benzoic acid (3809.3 mg, 12.44 mmol), 4-dimethylaminopyridine (68.3 mg, 0.56 mmol), EDAC (2104.2 mg, 13.55 mmol) in CH₂Cl₂ (42 mL), was added (1S,2S)-(−)-1,2-diphenylethylenediamine (1202.4 mg, 5.66 mmol), and the reaction stirred at room temperature overnight. Diethyl ether (80 mL) was added and the organics washed sequentially with 10% HCl (3×80 mL), water (1×80 mL), saturated aqueous NaHCO₃ (2×80 mL), 1 M NaOH (1×80 mL) and brine (1×80 mL). the organic layer was dried over MgSO₄, filtered and the solvent removed under reduced pressure. The residue was purified by flash column chromatography over silica gel eluting with EtOAc: Hex. The obtained white solid was redissolved in THF and dried with CaH₂ under N₂. The mixture was filtered, and the solvent removed under reduced pressure to recover the title compound (895 mg, 20%) as a white solid.

¹H NMR: (300 MHz; CDCl₃) δ 5.24 (dd, 2H), 6.76-6.80 (m, 6H), 6.92-7.21 (m, 32H), 7.49-7.53 (m, 2H).

³¹P{¹H}NMR: (121 MHz; CDCl₃) 5-10.15.

### Example 8

### ^{tBu}PHOX

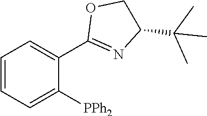

This ligand was synthesized in three steps according to literature procedure, including the synthesis of 2-diphenylphosphino-benzonitrile, (+)—{(4S)-4-tert-Butyl-4,5-dihydro-2-[2′-(diphenylphosphino)phenyl]oxazole}zinc(II) dichloride and the desired tBuPHOX ligand.

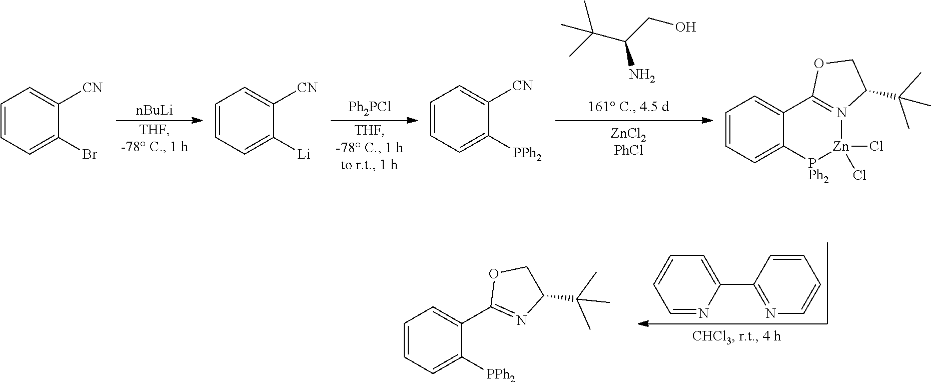

The synthesis of 2-diphenylphosphino-benzonitrile was performed under N₂ atmosphere. To a 100 mL Schlenk flask was added 2-bromobenzonitrile (2.73 g, 15 mmol) and 30 mL THF. The flask was kept at -78° C., followed by the slow addition of 2.5 M nBuLi (6 mL, 15 mmol). The reaction mixtures were stirred for one hour. Chlorodiphenylphosphine (2.83 mL, 15 mmol) in 5 mL THF was then added to the flask, followed by another one-hour stirring. The reaction was gradually warmed up to room temperature and stirred for one hour. The solution was diluted with 80 mL of diethyl ether. The organic layer was then washed with 70 mL water and 70 mL brine, respectively, followed by the addition of anhydrous MgSO₄. The crude product was obtained after the solvent evaporation under vacuum. The white solid was collected after recrystallization from hot methanol (1390 mg, 32%). In a glovebox, 2-diphenylphosphino-benzonitrile (820 mg, 2.85 mmol), ZnCl₂ (499 mg, 3.66 mmol), L-tert-Leucinol (423.4 mg, 3.61 mmol) and 12 mL chlorobenzene were added to a 4-dram vial and stirred at 160° C. for 4.5 days. The resulting mixtures were transferred to a silica plug and eluted with 600 mL ethyl acetate. The crude product was obtained after solvent evaporation. (+)—{(4S)-4-tert-Butyl-4,5-dihydro-2-[2′-(diphenylphosphino)phenyl]oxazole}zinc(II) dichloride was recrystallized from chloroform/TBME as white powders (316.5 mg, 21%). (+)—{(4S)-4-tert-Butyl-4,5-dihydro-2-[2′-(diphenylphosphino)phenyl]oxazole}zinc(II) dichloride (221.1 mg, 0.42 mmol), 2,2′-bypiridine (65 mg, 0.42 mmol) and 4 mL chloroform were added to a 2-dram vial and stirred at room temperature for 4 hours. The reaction mixtures were transferred to a silica plug and eluted with 50 mL chloroform. The solvent was evaporated to yield a colorless and oily product (140 mg, 86%).

To a solution of 2-bromobenzonitrile (2.73 g, 15 mmol) in THE (30 mL) at −78° C., was added nBuLi (2.5 M in hexanes, 6 mL, 15 mmol) and the reaction stirred for 1 h. a solution of chlorodiphenylphosphine (2.83 mL, 15 mmol) in THE (5 mL) was then added and the reaction stirred for a further 1 hour at −78° C., before being allowed to warm to room temperature and stirred at that temperature for 1 h. Diethyl ether (80 mL) was added, and the organic layer washed with water (70 mL) and brine (70 mL). the organic layer was dried over MgSO₄, filtered and the solvent removed under reduced pressure. The residue was purified by recrystallization from hot methanol to recover 2-diphenylphosphino-benzonitrile (1.39 g, 32%) as a white solid.

To a solution of 2-diphenylphosphino-benzonitrile (820 mg, 2.85 mmol), in chlorobenzene (12 mL) was added ZnCl₂ (499 mg, 3.66 mmol) and L-tert-Leucinol (423.4 mg, 3.61 mmol), and the reaction heated at 160° C. for 4.5 days. The reaction was passed through a pad of silica and washed with EtOAc (600 mL) and the solvent removed under reduced pressure. The residue was purified by recrystallization from hot chloroform/TBME to recover (+)—{(4S)-4-tert-Butyl-4,5-dihydro-2-[2′-(diphenylphosphino)phenyl]oxazole}zinc(II) (316.5 mg, 21%) as a white solid.

To a solution of (+)—{(4S)-4-tert-Butyl-4,5-dihydro-2-[2′-(diphenylphosphino)phenyl]oxazole}zinc(II) (221.1 mg, 0.42 mmol) in CHCl₃ (4 mL) was added 2,2′-bypiridine (65 mg, 0.42 mmol) and the reaction was stirred at room temperature for 4 h. The reaction was passed through a pad of silica and washed with CHCl₃ (50 mL) and the solvent removed under reduced pressure to recover the tBu-PHOX ligand (140 mg, 86%) as a white solid.

¹H NMR: (300 MHz; CD₂Cl₂) δ 0.76 (s, 9H, C(CH₃)₃), 3.85 (dd, 1H, CH₂), 4.02 (t, 1H, CH), 4.13 (dd, 1H, CH₂), 6.90 (m, 1H, Ar—H), 7.17-7.38 (m, 12H, Ar—H), 7.90 (m, 1H, Ar—H)

³¹P{¹H}NMR: (121 MHz; CD₂Cl₂) 5-7.03

### Example 9

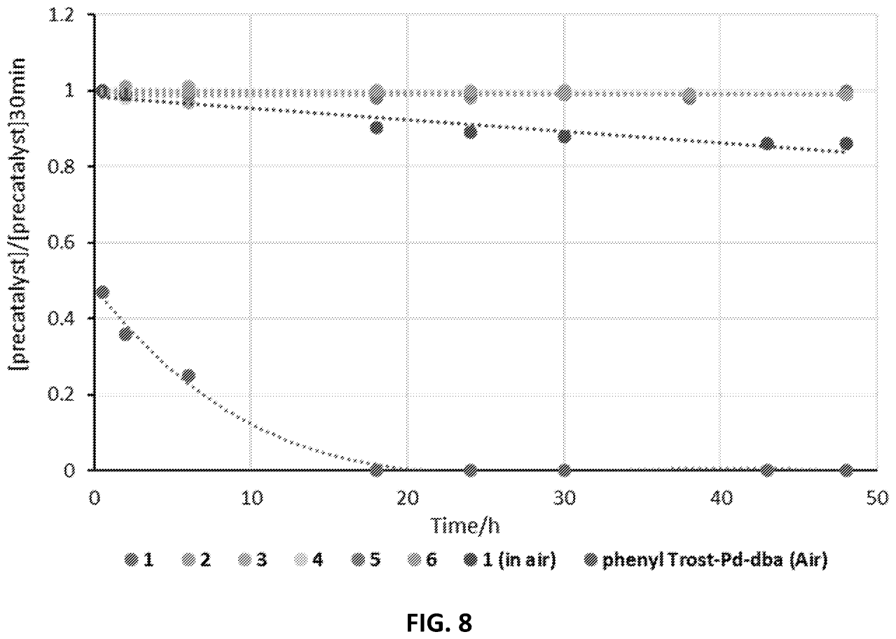

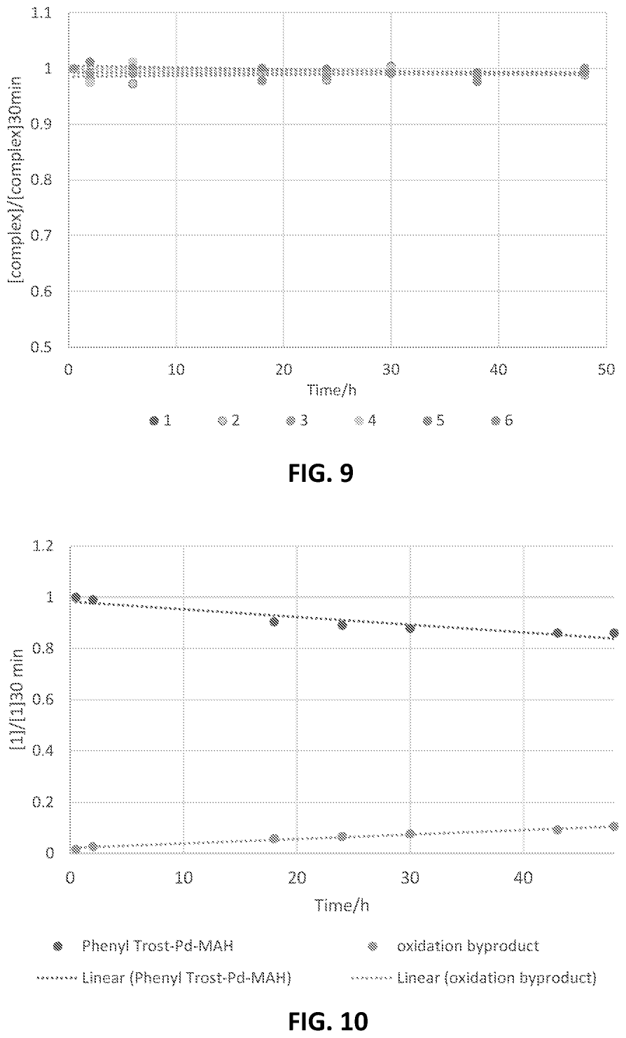

Solution Stability—General procedure: Precatalyst 1 (17.3 mg, 0.019 mmol) was dissolved in 0.7 mL THF. A capillary containing PPh₃ in C₆D₆ was used as an internal standard. Initial ³¹P NMR spectra were obtained for each solution after 30 minutes (500 MHz). Subsequent ³¹P NMR spectra were obtained at 2, 6, 18, 24, 30, 38, and 48 hours. Results are shown in FIG. 8 and summarized in Table 5; FIG. 9 shows a normalized plot for the results obtained from this example. With solutions prepared under N₂, the concentrations of all six complexes remain unchanged. The stability of a THE solution of precatalyst 1 after exposure to air also was evaluated. After 48 hours, there is still >80% of 1 intact, with the mass balance comprised of the oxidation product [PNNP]Pd″. In stark contrast, a mixture of (S,S)-^{Ph}DACH and Pd₂dba₃-CHCl₃ is 50% oxidized after only 30 minutes and is completely converted to [PNNP]Pd″ within 18 hours. Precatalyst 1 is indefinitely stable when stored as a solid under N₂ at room temperature and is also stable for weeks as a solid under air. Thus, precatalysts 1-6 can be handled and used without the need for a glovebox. As a matter of fact, all of the catalytic evaluation was carried out by weighing the precatalysts under air without any specific precaution.

TABLE 5
Solution stability of six chiral Pd complexes over
48 hours. The concentrations of complexes in THF
over 48 hours are normalized to assess stability.

[complex]/[complex] 30 minutes

| Time/h | 1 | 2 | 3 | 4 | 5 | 6 |
|---|---|---|---|---|---|---|
| 0.5 | 1 | 1 | 1 | 1 | 1 | 1 |
| 2 | 0.99 | 0.98 | 0.98 | 0.98 | 1.01 | 0.99 |
| 6 | 0.99 | 0.97 | 1 | 1.01 | 1 | 0.99 |
| 18 | 1 | 1 | 0.99 | 0.99 | 1 | 0.98 |
| 24 | 0.99 | 0.98 | 0.99 | 0.99 | 1 | 0.99 |
| 30 | 1 | 1 | 0.99 | 1 | 0.99 | 0.99 |
| 38 | 0.99 | 0.99 | 0.99 | 0.98 | 0.99 | 0.98 |
| 48 | 0.99 | 0.99 | 0.99 | 0.99 | 0.99 | 1 |

### Example 10

Stability of Precatalyst 1 in air—(S,S)-^{Ph}DACH-Pd-MAH-Pd-MAH (1, 14.9 mg, 0.017 mmol) was dissolved in 0.6 mL THF. Capillary comprising PPh₃ and C₆D₆ was used as internal standard. Initial ³¹P NMR spectra were obtained for each solution after 30 minutes (600 MHz). Subsequent ³¹P NMR spectra were obtained at 2, 18, 24, 30, 43, and 48 hours. Results also included in Table 5 and also are summarized graphically in FIG. 10.

TABLE 6
Solution stability of 1 exposed to air. Normalized [1] and
byproduct formation in THF over 48 hours to assess stability.

| Time/h | ^{Ph}DACH-Pd-MAH (1) | Oxidized Byproduct |
|---|---|---|
| 0.5 | 1 | 0.016 |
| 2 | 0.99 | 0.026 |
| 18 | 0.90 | 0.058 |
| 24 | 0.89 | 0.066 |
| 30 | 0.88 | 0.077 |
| 43 | 0.86 | 0.092 |
| 48 | 0.86 | 0.11 |

### Example 11

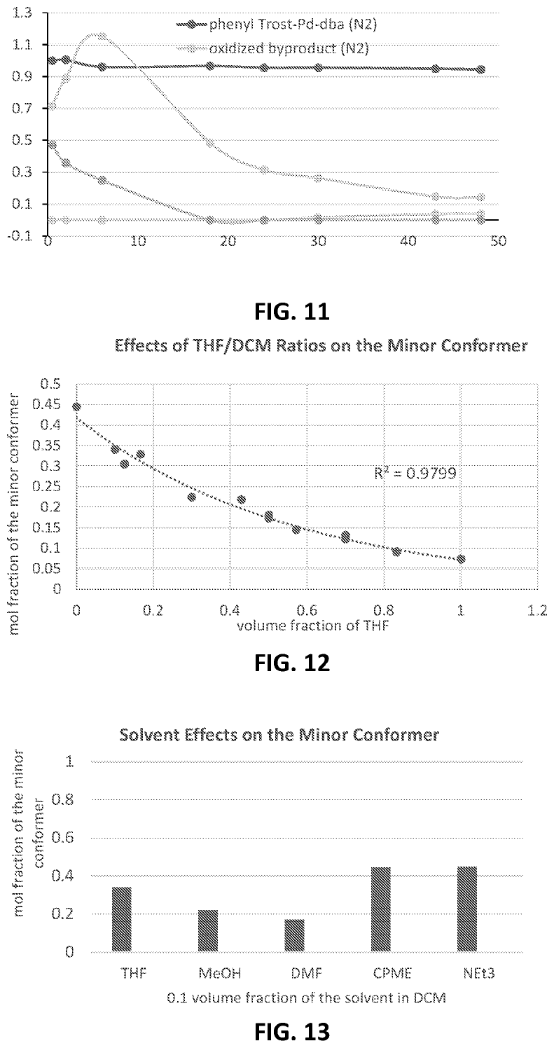

Stability of (S,S)-^{Ph}DACH-Pd-dba in N₂ and air—Pd₂dba₃CHCl₃ (12.9 mg, 0.0125 mmol), (S,S)-^{Ph}DACH (16.8 mg, 0.0243 mmol), and 1.2 mL THF were added to a 1-dram vial under N₂ atmosphere. The mixtures were stirred at room temperature for one hour. The solution was evenly split into two and transferred to two different NMR tubes with capillaries comprising PPh₃ and C₆D₆ as the internal standard. Initial ³¹P NMR spectra were obtained for each solution after 30 minutes (200 MHz). One solution was exposed to the air after the initial ³¹P{¹H}NMR spectrum was taken. Subsequent ³¹P NMR spectra were obtained at 2, 6, 18, 24, 30, 43, and 48 hours. Results also provided in Table 7 and also are summarized graphically in FIG. 11.

TABLE 7
Solution stability of ^{Ph}DACH-Pd-dba in the air and
N₂. Normalized [^{Ph}DACH-Pd-dba] and Pd(II)
oxidation byproduct formation in THF over 48 hours.

| | | Oxidized | | Pd(II) Oxidized |
|---|---|---|---|---|
| Time/ | ^{Ph}DACH-Pd-dba | Byproduct | ^{Ph}DACH-Pd-dba | Byproduct |
| h | (N₂) | (N₂) | (air) | (air) |
| 0.5 | 1 | 0.00 | 0.47 | 0.71 |
| 2 | 1.01 | 0.00 | 0.36 | 0.89 |
| 6 | 0.96 | 0.00 | 0.25 | 1.15 |
| 18 | 0.97 | 0.00 | 0.00 | 0.48 |
| 24 | 0.96 | 0.00 | 0.00 | 0.31 |
| 30 | 0.96 | 0.016 | 0.00 | 0.26 |
| 43 | 0.95 | 0.038 | 0.00 | 0.15 |
| 48 | 0.94 | 0.039 | 0.00 | 0.14 |

### Example 12

Investigation of solvent effect on conformer ratio—Two sets of ³¹P{¹H}NMR peaks were observed when dissolving (S,S)-^{Ph}DACH-Pd-MAH in DCM-d2. Further investigation was performed on the different ratios between THF and DCM. In a 1-dram vial, (S,S)-^{Ph}DACH-Pd-MAH (1, 19 mg, 0.021 mmol) was dissolved in 1.2 mL of DCM. 0.6 mL of the solution was transferred to an NMR tube, and a capillary comprising PPh₃ in C₆D₆ was used as an internal standard. Initial ³¹P spectrum was obtained (500 MHz). The solution concentration was diluted twice, four times, and eight times by the subsequent addition of (3×) 0.6 mL DCM to the vial. Results are summarized in Table 8.

TABLE 8
Changes of two conformers in ³¹P{¹H}
NMR spectra in different concentrations.

| | major conformer | minor conformer | major/minor |
|---|---|---|---|
| 1× | 2.0067 | 1.5406 | 1.302544 |
| 2× | 2.0004 | 1.6272 | 1.229351 |
| 4× | 1.9326 | 1.4185 | 1.362425 |
| 8× | 2.0751 | 1.4338 | 1.447273 |

(S,S)-^{Ph}DACH-Pd-MAH (1, 19.9 mg, 0.022 mmol) was dissolved in 0.3 mL of THF, followed by the addition of 0.3 mL of DCM. Capillary comprising PPh₃ in C₆D₆ was used as an internal standard. Initial ³¹P{¹H}NMR spectrum starting from THF/DCM (1:1) was obtained (500 MHz). Different amounts of THF were subsequently added to adjust the DCM/THF ratio to 3:4, 3:7, 1:5, 1:7, 1:9. ³¹P{¹H}NMR spectra were obtained at each ratio.

Another experiment was performed in a similar way. Different amounts of DCM were subsequently added to adjust the THF/DCM ratio to 3:4, 3:7, 1:5, 1:7. ³¹P{¹H}NMR spectra were obtained at each ratio. Results are summarized in FIG. 12.

(S,S)-^{Ph}DACH-Pd-MAH (1, 10 mg, 0.011 mmol) was dissolved in 0.9 mL of DCM, followed by the addition of 0.1 mL of THF. Capillary comprising PPh₃ in C₆D₆ was used as an internal standard. Similarly, other samples were prepared using 0.1 mL of cosolvents MeOH, DMF, CPME, NEt₃. ³¹P{¹H}NMR spectra were obtained. Results are summarized in FIG. 13. A 10:1 DCM/THF mixture gives a 65:35 ratio of 1-exo to 1-endo, 10:1 DCM/DMF or DCM/MeOH mixtures give roughly a 80:20 ratio in favor of the 1-exo conformer, whereas addition of less polar co-solvents such as CPME and NEt₃ results in no change to the conformer ratio.

The solvent effect on conformer ratios observed for precatalyst 1 are explained as follows. In relatively non-polar solvents (e.g., DCM), 1-endo and 1-exo conformers are similar in energy; however, with the addition of a more polar and hydrogen-bond-accepting solvent, intermolecular hydrogen bonding between an N—H bond in the ligand and the solvent will stabilize the 1-exo conformation.

### Example 13

General Procedure for Catalyst Activation—In the glovebox, a 1-dram vial was added ^{DMP}DAB-Pd-MAH (5.6 mg, 0.012 mmol), (S,S)-^{Ph}DACH (8.3 mg, 0.012 mmol), 0.6 mL THF, and corresponding additives.

The mixtures were stirred at room temperature for one hour. The reaction solution was finally transferred to an NMR tube with a capillary comprising PPh₃ in C₆D_{6.} ³¹P NMR (200 MHz) spectrum was obtained, and the same capillary was used in all activation tests.

Additives and corresponding % consumption of precatalyst 1 are listed below in Table 9.

TABLE 9
Additives of activation tests and the
corresponding % consumption of 1.

| | Consumption |
|---|---|
| Additives | of 1 |
| 2 equiv. KOtBu | 100% |
| 2 equiv. KOAc | 0% |
| 21 equiv. BSA + 3 equiv. KOAc | 15% |
| 1.3 equiv. 1,3-diphenylisobenzofuran | 2% |
| 20 equiv. dimethyl malonate + 20 equiv. NaH | 59% |
| 21 equiv. dimethyl malonate + 21 equiv. NaH + 26 equiv. | 73% |
| BSA | |
| 21 equiv. dimethyl malonate + 21 equiv. BSA + 3 equiv. | 26% |
| KOAc | |

### Example 14

### Initial Catalytic Study for Malonation of Cyclohex-2-En-1-Yl Methyl Carbonate by DYKAT

In this example, malonate addition on racemic cyclohex-2-en-1-yl methyl carbonate was evaluated. Under standard Pd-AAA conditions (e.g., 5 mol % Pd and a Pd:L ratio of 1:1.5), the allylation proceeded efficiently and selectively (96% yield, 95% ee, Table 10, entry 2). Gratifyingly use of ^{DMP}DAB-Pd-MAH as a Pd source offered similar results (Table 10, entry 3). Strikingly, reducing the Pd:L ratio to 1:1 resulted in no reaction (Table 10, entry 4). As the absence of ligand shows that Pd₂dba₃ alone does not catalyse the reaction (Table 10, entry 5), it is clear that an excess of ligand is required to generate the active catalytic species. In sharp contrast, the use of ^{Ph}DACH-Pd-MAH delivered the desired product in a comparable 86% yield and 96% ee (Table 10, entry 6).

Cyclohex-2-en-1-yl methyl carbonate: To a solution of cyclohex-2-ol (0.5 mL, 5.1 mmol, 1 equiv) and pyridine (2.4 mL, 30 mmol, 6 equiv) in CH₂Cl₂ (15 mL) at 0° C. was added methyl chloroformate (1 mL, 12.8 mmol, 2.5 equiv). The reaction was then stirred at room temperature for 18 hours. the reaction was quenched with 1 M HCl (15 mL) and the layers separated. The organic phase was then washed with saturated aqueous NaHCO₃, and brine, dried over MgSO₄, filtered and the solvent removed under reduced pressure. the residue was purified by flash column chromatography over silica gel eluting with Hexane and ethyl acetate to recover the title compound (755 mg, 4.8 mmol, 95%) as a colorless oil. 1H NMR (400 MHz, CDCl₃) δ 6.00-5.95 (1H, m), 5.77 (1H, m), 5.12 (1H, m), 3.77 (3H, s), 2.15-1.58 (6H, m). 13C NMR (100 MHz, CDCl₃) δ 155.7, 133.5, 125.1, 77.5, 77.2, 76.8, 72.1, 54.7, 28.4, 25.0, 18.7.

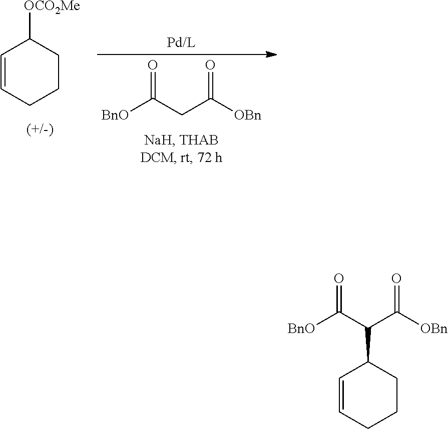

TABLE 10

| | Pd | Pd | L | | Yield | ee |
|---|---|---|---|---|---|---|
| Entry | source | (mol %) | (mol %) | Pd:L | % | % |
| 1 | [PdCl(allyl)]₂ | 5 | 7.5 | 1:1.5 | 86 | 95 |
| 2 | Pd₂dba₃ | 5 | 7.5 | 1:1.5 | 96 | 95 |
| 3 | ^{DMP}DAB-Pd-MAH | 5 | 7.5 | 1:1.5 | 86 | 95 |
| 4 | Pd₂dba₃ | 5 | 5 | 1:1 | 0 | — |
| 5 | Pd₂dba₃ | 5 | 0 | 1:0 | 0 | — |
| 6 | ^{Ph}DACH-Pd-MAH | 5 | — | 1:1 | 86 | 95 |

L = (S,S)-DACH-Phenyl. THAB = tetrahexylammonium benzoate. Reactions conducted on a 0.3 mmol scale, with 2 equivalents of dibenzyl malonate, 2.5 equivalents of NaH, and 3 equivalents of THAB in CH₂Cl₂ [0.05M].

of NaH (60% in mineral oil, 12 mg, 0.52 mmol, 3.1 equiv. triturated with hexane prior to use to remove the mineral oil) in CH₂Cl₂ (2 ml) was added dibenzyl malonate² (140 μL, 0.5 mmol, 2 equiv) and stirred at room temperature for 5 mins. THAB (225 mg, 0.52 mmol, 3.1 equiv.) in CH₂Cl₂ (0.5 ml) was then added and a suspension formed. In a separate vessel, either Pd source and (S,S)-DACH-Phenyl (in respective mol %) or DACH^{Ph}-Pd-MAH (4.4 mg, 0.05 mmol, 2 mol %) was dissolved in 3.5 mL CH₂Cl₂ and stirred for 30 mins at room temperature after which time, cyclohex-2-en-1-yl methyl carbonate (39 mg, 0.25 mmol, 1 equiv) was added. The suspension of dibenzyl malonate was then added by syringe dropwise at room temperature and the reaction stirred for 24 hours. The reaction was quenched with H₂O (10 ml) and the layers separated, the aqueous phase was extracted with CH₂Cl₂ (3×10 ml), the combined organics were dried over Mg₂SO₄, filtered and the solvent removed under reduced pressure. The residue was purified by silica gel column chromatography eluting with Hexane and EtOAc (100:0 to 95:5) to recover the title compound. ¹H NMR (400 MHz, CDCl₃) δ 7.37-7.28 (10H, m), 5.75 (1H, m), 5.55 (1H, m), 5.20-5.12 (4H, m), 3.40 (1H, d, J9.1), 2.96 (1H, m), 2.01-1.93 (2H, m), 1.82-1.65 (2H, m), 1.61-1.49 (1H, m), 1.39 (1H, m). ¹3C NMR (100 MHz, CDCl₃) δ 168.4, 168.3, 135.6, 135.5, 129.8, 128.7, 128.4, 128.4, 128.3, 128.3, 127.5, 77.5, 77.2, 76.8, 67.1, 57.2, 35.6, 26.7, 25.1, 21.1.

### Example 16

### Desymmetrization of Meso Bis-Acetates

In this example, the desymmetrization of meso-2-en-1,4-diol diester was evaluated using the Pd/^{Ph}DACH system. Under the same standard Pd-AAA conditions, the desymmetrization was sensitive to the nature of the Pd source with (PdClallyl)₂, Pd₂dba₃, and ^{DMP}DAB-Pd-MAH providing variable yields (35-65%) albeit roughly similar ees (95-98%). ^{Ph}DACH-Pd-MAH at 2 mol % provided the desired product and maintained an excellent selectivity (68% yield, 98% ee, Table 11, entry 8), whereas other evaluated catalysts/precatalysts currently used in the art exhibited lower yields and/or selectivity.

Cis-cyclohex-2-ene-1,4-diyl diacetate: To a vial charged with PdOAc₂ (70 mg, 0.3 mmol), LiCl (52 mg, 0.1 mmol), LiOAc (2.15 g, 32.6 mmol), benzoquinone (160 mg, 1.48 mmol, freshly sublimed), and MnO₂ (680 mg, 7.82 mmol), was added acetic acid (5 mL). A solution of Cyclohexa-1,3-diene (500 mg, 6.2 mmol) in Hexane (10 mL) was then added and the mixture vigorously stirred at room temperature for 24 hours. the mixture was filtered through celite and brine (30 mL) was added to the filtrate. The mixture was extracted with hexane (3×30 mL), the combined organics dried over MgSO₄, filtered, and the solvent removed under reduced pressure. The residue was purified by silica gel column chromatography eluting with Hexane and EtOAc (100:0 to 90:10) to recover the title compound (921 mg, 4.65 mmol, 75%) as a colorless oil. ¹H NMR (400 MHz, CDCl₃) δ 5.90 (2H, s), 5.22 (2H, bs), 2.06 (6H, s), 1.81-1.95 (4H, m). ¹³C NMR (100 MHz, CDCl₃) δ 170.7, 130.4, 67.4, 25.0, 21.4.

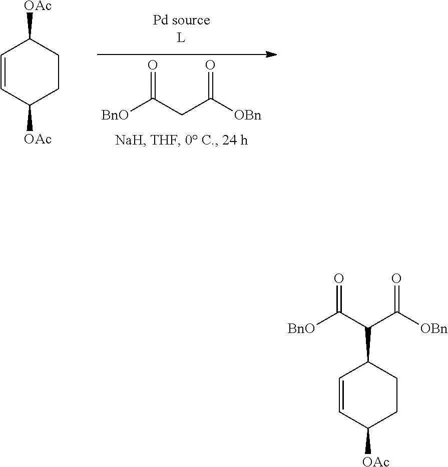

TABLE 11

| | Pd | Pd | L | | Yield | ee |
|---|---|---|---|---|---|---|
| Entry | source | (mol %) | (mol %) | Pd:L | % | % |
| 1 | [PdCl(allyl)]₂ | 5 | 7.5 | 1:1.5 | 56 | 95 |
| 2 | Pd₂dba₃ | 5 | 7.5 | 1:1.5 | 35 | 96 |
| 3 | ^{DMP}DAB-Pd-MAH | 5 | 7.5 | 1:1.5 | 65 | 98 |
| 4 | [PdCl(allyl)]₂ | 2 | 2 | 1:1 | 46 | >99 |
| 5 | Pd₂dba₃ | 2 | 3 | 1:1.5 | 24 | 98 |
| 6 | Pd₂dba₃ | 2 | 2 | 1:1 | 15 | 92 |
| 7 | ^{DMP}DAB-Pd-MAH | 2 | 2 | 1:1 | 30 | 92 |
| 8 | ^{Ph}DACH-Pd-MAH | 2 | — | 1:1 | 68 | 98 |

L = (S,S)-^{Ph}DACH. Reactions run on a 0.3 mmol scale with 1.2 equivalents of malonate, 1.5 equivalents of NaH in THF [0.1M].

Dibenzyl 2-((1S,4R)-4-acetoxycyclohex-2-en-1-yl)malonate: To a solution of Pd source (indicated mol %) and (S,S)-^{Ph}DACH (if required, in indicated mol %) in THE (0.5 ml) having stirred at room temperature for 30 mins, was added cis-2-cyclohexenyl-1,4-diacetate (0.3 mmol, 60 mg, 1 equiv.). In a separate vessel, to a suspention of NaH (13 mg, 0.36 mmol, 1.2 equivs.) in THE (1 mL) was added dibenzyl malonate (105 mg, 0.36 mmol, 1.2 equivs.) and the solution stirred at room temperature for 5 mins. The solution of catalyst was cooled to 0° C. before addition of the malonate solution via syringe. The reaciton was stirred at 0° C. for 24 hours before diluting with water (10 ml) and extracting with CH₂Cl₂. The combined organics were dried over Mg₂SO₄, filtered and the solvent removed under reduced preasure. The residue was purified by silica gel column chromatography eluting with Hexane and EtOAc (100:0 to 95:5) to recover the title compound. ¹H NMR (400 MHz, CDCl₃) δ 7.36-7.27 (10H, m), 5.86-5.77 (2H, m), 5.20-5.12 (5H, m), 3.43 (2H, d, J9.1), 2.90 (2H, m), 2.02 (3H, s), 1.88-1.66 (3H, m), 1.58-1.48 (3H, m). ¹³C NMR (100 MHz, CDCl₃) δ 170.7, 168.0, 167.9, 135.4, 135.4, 133.5, 128.7, 128.5, 128.5, 128.3, 127.1, 67.4, 66.4, 56.4, 35.6, 27.1, 22.4, 21.4.

### Example 17

Decarboxylative Pd-AAA constitute a broad part of the field of Pd-AAA. In this example, Pd sources in the Pd-DAAA of allyl (2-phenyl-cyclohexyl) carbonate were screened. Common Pd(0) sources delivered the allylated cyclohexanone in similar yields (77-86%) and enantioselectivities (81-83%) although a significant drop in reactivity was observed with [Pd(allylCl]2 (Table 12, entries 1-3). A lower catalyst loading with Pd₂dba₃ did not seem to greatly affect the reaction outcome (Table 12, entry 4). The detrimental effect was again the improper Pd to ligand stoichiometry, impeding the activity of the catalyst, significantly reducing the yield from 77% to 39% with Pd₂dba₃ and from 86% to 43% with ^{DMP}DAB-Pd-MAH. This detriment was resolved by the use of the preformed Pd(0) precursor complex, ^{Ph}ANDEN-Pd-MAH, which at 2 mol % provided the product in excellent yield (86%) and up to 81% ee.

2-phenylcyclohexan-1-one: Cyclohexanone (1.38 ml, 13.2 mmol, 1.1 equiv) and bromobenzene (1.35 ml, 12 mmol, 1 equiv) were added to a solution of KO^{t}Bu (2 g, 18 mmol, 1.5 equiv) in THE (10 ml). In a separate vessel, PdOAc₂ (54 mg, 0.24 mmol, 2 mol %) was dissolved in THE (1 ml) and P^{t}Bu₃ (72 mg, 0.36 mmol, 2.5 mol %) was added, the resulting red solution was added to the starting materials and the reaction heated at 60° C. for 3 hours. The reaction was cooled to room temperature and filtered through celite washing with Et₂O (50 ml). The organics were then washed with water, saturated NaHCO₃, and brine, dried over MgSO₄, filtered and the solvent removed under reduced pressure. The crude material was purified by silica gel column chromatography eluting with Hexane and Et₂O (95:5) to recover the title compound (1.2 g, 0.69 mmol, 58%) as a white solid. The product can be further purified if desired by dissolving in the minimum volume of CH₂Cl₂ and adding dropwise to Hexane at 0° C., the precipitate was filtered to obtain the title compound as a white solid. ¹H NMR (400 MHz, CDCl₃) δ 7.34 (1H, m), 7.28-7.23 (1H, m), 7.16-7.12 (2H, m), 3.61 (1H, dd, J12.1, 5.4), 2.58-2.41 (2H, m), 2.32-2.23 (1H, m), 2.20-1.97 (3H, m), 1.90-1.76 (2H, m). ¹3C NMR (100 MHz, CDCl₃) δ 210.4, 138.9, 128.7, 128.5, 127.0, 57.5, 42.3, 35.2, 28.0, 25.5.

Allyl (3,4,5,6-tetrahydro-[1,1′-biphenyl]-2-yl) carbonate: To a solution of 2-phenylcyclohexan-1-one (560 mg, 3.2 mmol, 1 equiv) in THE (1.6 mL) was added NaH (141 mg 3.5 mmol, 1.1 equiv., triturated with hexane prior to use to remove the mineral oil), and TMEDA (0.48 mL). The mixture was heated at reflux for 1 hour before being cooled to 0° C. Allyl chloroformate (0.35 mL, 3.2 mmol, 1.1 equiv) in THE (1.6 mL) was cooled to 0° C. and added to the 2-phenylcyclohexan-1-one solution via cannula at 0° C. The reaction was stirred at 0° C. for 15 mins before being quenched with sat. NH₄Cl (20 mL) and extracted with ether (3×20 mL). The organics were dried over MgSO₄, filtered, and the solvent removed under reduced pressure. The residue was purified by silica gel column chromatography eluting with Hexane and Diethyl Ether (95:5) to recover the title compound (405 mg, 1.6 mmol, 49%) as a colorless oil. Note: the product co-eluted on the column with diallylcarbonate, an impurity that was removed under reduced pressure at 50° C. ¹H NMR (400 MHz, CDCl₃) δ 7.33-7.28 (2H, m), 7.26-7.19 (3H, m), 5.79 (1H, ddt, J 17.2, 10.5, 5.6), 5.22-5.15 (2H, m), 4.51 (2H, dt, J5.6, 1.4), 2.37 (4H, m), 1.88-1.73 (4H, m). ¹³C NMR (100 MHz, CDC₃) δ 153.0, 143.5, 138.9, 131.5, 128.3, 127.8, 127.0, 126.0, 118.7, 68.6, 30.3, 27.2, 23.0, 22.7.

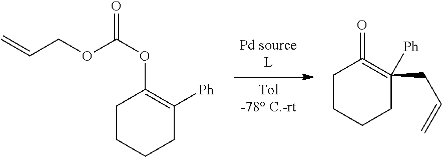

TABLE 12

| | Pd | Pd | L | | Time | Yield | ee |
|---|---|---|---|---|---|---|---|
| Entry | source | (mol %) | (mol %) | Pd:L | (h) | % | % |
| 1 | [PdCl(allyl)]₂ | 5 | 7.5 | 1:1.5 | 24 | 21 | 81 |
| 2 | Pd₂dba₃ | 5 | 7.5 | 1:1.5 | 0.5 | 86 | 83 |
| 3 | ^{DMP}DAB-Pd-MAH | 5 | 7.5 | 1:1.5 | 0.5 | 84 | 81 |
| 4 | Pd₂dba₃ | 2 | 3 | 1:1.5 | 0.5 | 77 | 83 |
| 5 | Pd₂dba₃ | 2 | 2 | 1:1 | 24 | 39 | 82 |
| 6 | ^{DMP}DAB-Pd-MAH | 2 | 3 | 1:1.5 | 1 | 86 | 82 |
| 7 | ^{DMP}DAB-Pd-MAH | 2 | 2 | 1:1 | 24 | 43 | 81 |
| 8 | ^{Ph}ANDEN-Pd-MAH | 2 | — | 1:1 | 1 | 87 | 81 |

L = (S,S)-ANDEN-Phenyl. Reactions run on a 0.2 mmol scale in Tol [0.1M].

(R)-2-allyl-2-phenylcyclohexan-1-one: The palladium source (indicated mol %) and (S,S)-^{Ph}ANDEN ligand (if required at indicated mol %) were dissolved in toluene (1 mL) and stirred at room temperature for 20 mins before being cooled to −78° C. Allyl-(3,4,5,6-tetrahydro-[1,1′-biphenyl]-2-yl) carbonate (52 mg, 0.2 mmol) in toluene (1 mL) was cooled to −78° C. and then added to the catalyst mixture. The reaction was stirred at −78° C. for 10 mins before being removed from the cooling bath and allowed to warm to room temperature over ˜5 mins. Once the starting material had been consumed (indicated time) as judged by TLC, or after 24 hours, the reaction was diluted with ether (20 mL) and washed with brine. The organic layer was dried over MgSO₄, filtered, and the solvent removed under reduced pressure. The residue was purified by silica gel column chromatography eluting with Hexane and Diethyl Ether (95:5) to recover the title compound as a colorless oil. ¹H NMR (400 MHz, CDCl₃) δ 7.34 (2H, m), 7.26-7.22 (1H, m), 7.15 (2H, m), 5.45 (1H, dddd, J17.0, 10.2, 7.9, 6.8), 4.89 (2H, m), 2.71-2.63 (1H, m), 2.54-2.39 (2H, m), 2.39-2.24 (2H, m), 1.98-1.89 (1H, m), 1.82-1.61 (4H, m). ¹³C NMR (100 MHz, CDCl₃) δ 213.2, 140.7, 134.5, 128.9, 127.0, 126.9, 117.8, 76.8, 76.8, 57.0, 45.1, 40.3, 34.8, 28.4, 21.7.

### Example 18

In this example, the reaction of (E)-1,3-diphenylallyl acetate with dimethyl malonate is often considered as the model reaction when developing novel catalysts for Pd-AAA chemistry. Hence, ^{DMP}DAB-Pd-MAH was compared with the typical (PdClallyl)₂ and Pd₂dba₃ catalytic systems in the presence of PHOX-type ligands (Table 13). ^{DMP}DAB-Pd-MAH afforded the product in 81% yield and 97% ee after 24 hours. (PdClallyl)₂ and Pd₂dba₃ combined with PHOX ligands required pre-heating at 50° C. for about 1 hour to effectively form the active complex and if this activation step is not conducted then a detrimental loss of reactivity ensues (Table 13, entries 1, 2, 4, and 5). This highlights a great improvement in practicality of the ^{DMP}DAB-Pd-MAH catalyst, as ligand exchange occurs in less than 5 minutes at room temperature and hence retains activity with or without heating (Table 13, entries 3 and 6). Entry 7 further illustrates the practicality of the precatalyst embodiments of the present disclosure as they can be added as a solid to the reaction mixture without inducing any erosion of either the reactivity or the selectivity. Entries 13 and 14 (Table 13), where a ‘hard’ potassium enolate nucleophile was engaged in THF, fully recovered the reactivity of both ^{Pr}PHOX—Pd-MAH and f^{Bu}PHOX—Pd-MAH, with full conversion after 1 hour and complete recovery of the selectivity in the case of BUPHOX—Pd-MAH.

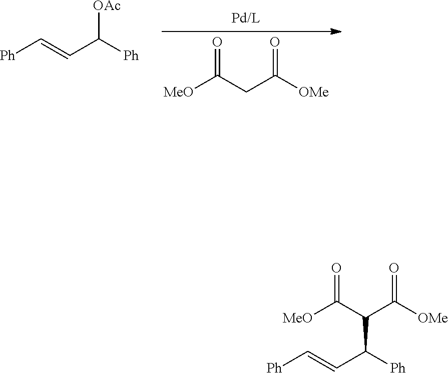 [SMILES: CC(=O)OC(/C=C/c1ccccc1)c1ccccc1 ; COC(=O)CC(=O)OC ; COC(=O)C(C(=O)OC)[C@H](/C=C/c1ccccc1)c1ccccc1]

### Conditions:

A: BSA (3 equiv), KOAc (2 mol %)
CH₂Cl₂ [0.3] M

B: KH (3 equiv)
THF [0.1] M

TABLE 13

| | Pd | Pd | L | | | Time | Yield | ee |
|---|---|---|---|---|---|---|---|---|
| Entry | source | (mol %) | (mol %) | Pd:L | Cond | (h) | % | % |
| 1^{a} | [PdCl(allyl)]₂ | 2 | (R)-^{iPr}PHOX | 1:1.25 | A | 1 | 93 | 95 |
| | | | (2.5) | | | | | |
| 2^{a} | Pd₂dba₃ | 2 | (R)-^{iPr}PHOX | 1:1.25 | A | 1 | 80 | 95 |
| | | | (2.5) | | | | | |
| 3^{a} | ^{DMP}DAB-Pd-MAH | 2 | (R)-^{iPr}PHOX | 1:1.25 | A | 24 | 81 | 97 |
| | | | (2.5) | | | | | |
| 4^{b} | [PdCl(allyl)]₂ | 2 | (R)-^{iPr}PHOX | 1:1.25 | A | 24 | 67 | 94 |
| | | | (2.5) | | | | | |
| 5^{b} | Pd₂dba₃ | 2 | (R)-^{iPr}PHOX | 1:1.25 | A | 24 | 59 | 97 |
| | | | (2.5) | | | | | |
| 6^{b} | ^{DMP}DAB-Pd-MAH | 2 | (R)-^{iPr}PHOX | 1:1.25 | A | 24 | 89 | 97 |
| | | | (2.5) | | | | | |
| 7^{c} | (S)-^{iPr}PHOX-Pd-MAH | 2 | — | 1:1 | A | 24 | 84 | −96 |
| 8^{a} | [PdCl(allyl)]₂ | 2 | (R)-^{tBu}PHOX | 1:1.25 | A | 1 | 89 | 96 |
| | | | (2.5) | | | | | |
| 9^{a} | ^{DMP}DAB-Pd-MAH | 2 | (R)-^{tBu}PHOX | 1:1.25 | A | 24 | 72 | 85 |
| | | | (2.5) | | | | | |
| 10^{c} | (S)-^{tBu}PHOX-Pd-MAH | 2 | — | 1:1 | A | 24 | 80 | −82 |
| 11^{d} | [PdCl(allyl)]₂ | 2 | (R)-^{iPr}PHOX | 1:1.25 | A | 2 | 94 | 96 |
| | | | (2.5) | | | | | |
| 12^{b} | [PdCl(allyl)]₂ | 2 | (R)-^{iPr}PHOX | 1:1.25 | B | 1 | 94 | 96 |
| | | | (2.5) | | | | | |
| 13^{c} | (S)-^{iPr}PHOX-Pd-MAH | 2 | — | 1:1 | B | 1 | 89 | −96 |
| 14^{c} | (S)-^{tBu}PHOX-Pd-MAH | 2 | — | 1:1 | B | 1 | 83 | −93 |

^{a}Pd and L heated at 50° C. for 1 hour prior to addition of SM.
^{b}Pd and L stirred at rt for 1 hour prior to addition of SM.
^{c}Pre-catalyst added as a solid to SM at rt.
^{d}5 mol % MAH added after ligand exchange, and reaction heated for an additional 1 hour before addition of SM at rt.

Condition A: Pd source (2 mol %) and PHOX ligand (2.5 mol % if required) in degassed CH₂Cl₂ (0.3 mL) stirred at room temperature for 2 hours, or was heated at 50° C. for 2 hours, or added as a solid as indicated. The catalyst solution was then allowed to cool to room temperature added to a mixture of (±)-(trans)-1,3-diphenylallyl acetate (76 mg, 0.3 mmol), dimethyl malonate (102 μL, 0.9 mmol), N,O-Bis(trimethylsilyl)acetamide (BSA, 220 μL, 0.9 mmol), and KOAc (1 mg, 0.006 mmol, 2 mol %) in degassed CH₂Cl₂ (0.7 mL). The reaction was stirred at room temperature until complete. The reaction was diluted with diethyl ether (20 mL) and washed with saturated NH₄Cl_{aq}. The organic layer was dried over Mg₂SO₄, filtered and the solvent removed under reduced pressure. The residue was purified by silica gel column chromatography eluting with Hexane and EtOAc (95:5) to recover the title compound.

Condition B: To a suspension of KH (30% in mineral oil, 100 mg, 0.75 mmol, 2.5 equiv, triturated with hexane prior to use to remove the mineral oil) in THE (2 mL) was added dimethyl malonate (102 μL, 0.9 mmol) dropwise. And the reaction stirred for 20 mins at room temperature. (±)-(trans)-1,3-diphenylallyl acetate (76 mg, 0.3 mmol) was then added. Separately Pd source (2 mol %) and PHOX ligand (2.5 mol % if required) in degassed THE (1 mL) stirred at room temperature for 2 hours, or was heated at 50° C. for 2 hours, or added as a solid to the reaction in THE (3 mL) as indicated. The catalyst solution was then added to the reaction at room temperature and stirred for the indicated time. H₂O (20 mL) was added and the mixture extracted with CH₂Cl₂. The combined organics were dried over MgSO₄, filtered, and the solvent removed under reduced pressure. The residue was purified by silica gel column chromatography eluting with Hexane and Diethyl Ether (95:5) to recover the title compound.

dimethyl (S,E)-2-(1,3-diphenylallyl)malonate: ¹H NMR (400 MHz, CDCl₃) δ 7.34-7.18 (10H, m), 6.49 (1H, d, J15.8), 6.34 (1H, dd, J15.7, 8.6), 4.28 (1H, dd, J10.7, 8.7), 3.96 (1H, d, J10.9), 3.71 (3H, s), 3.52 (3H, s). ¹³C NMR (100 MHz, CDCl₃) δ 168.3, 167.9, 140.3, 137.0, 132.0, 129.3, 128.9, 128.6, 128.0, 127.7, 127.3, 126.5, 57.8, 52.7, 52.6, 49.3.

### Example 19

Butadiene Monoxide Opening with Nitrogen Nucleophile

In this example, a Pd-AAA reaction involving a nitrogen-nucleophile was evaluated. The readily scalable amination of the racemic epoxide shown below has been utilized in several total syntheses and proved efficient even at low catalyst loading. In this example, ^{NAP}DACH-Pd-MAH provided the desired product in quantitative yield and a higher selectivity compared to classic systems (Table 14, comparing entry 4 with entries 2 and 3).

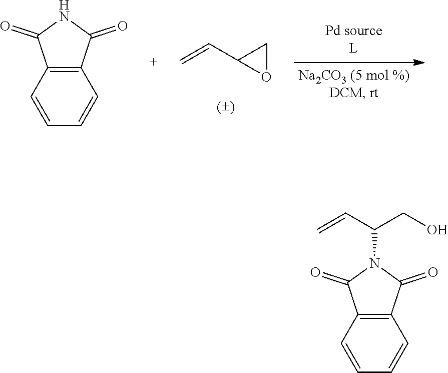

TABLE 14

| | Pd | Pd | L | | Yield | ee |
|---|---|---|---|---|---|---|
| Entry | source | (mol %) | (mol %) | Pd:L | % | % |
| 1 | [PdCl(allyl)]₂ | 0.8 | 1.2 | 1:1.5 | 99 | 72 |
| 2 | Pd₂dba₃ | 0.8 | 1.2 | 1:1.5 | 82 | 64 |
| 3 | ^{DMP}DAB-Pd-MAH | 0.8 | 1.2 | 1:1.5 | 84 | 82 |
| 4 | ^{NAP}DACH-Pd-MAH | 0.8 | — | 1:1 | 99 | 81 |

L = (S,S)-DACH-Napthyl. Reactions run on a 1 mmol scale with respect to the epoxide, with 1.05 equivalents of phthalimide, and 0.05 equivalents Na₂CO₃ in CH₂Cl₂ [0.125M].

2-(1-Hydroxybut-3-en-2-yl)isoindoline-1,3-dione: A solution of Pd source (indicated mol %) and (S,S)-DACH-Napthyl (Indicated mol %), and Na₂CO₃ (5.3 mg, 0.012 mmol) in CH₂Cl₂ (8 mL) was stirred at room temperature for 30 mins, to which was added Phthalimide (147 mg, 1.05 mmol), and 3,4-Epoxy-1-butene (81 μL, 1 mmol). The reaction was stirred at room temperature overnight. The solvent was removed in under reduced pressure, and the residue purified by a short flash column chromatography over silica gel eluting with EtOAc: Hex 1:9 to recover the title compound eluting with Hexane:Et₂O (50:50) to recover the title compound as a colorless solid. ¹H NMR (400 MHz, CDCl₃) δ 7.88-7.82 (2H, m), 7.76-7.70 (2H, m), 6.21-6.11 (1H, m), 5.33-5.24 (2H, m), 4.97-4.89 (1H, m), 4.14 (1H, dt, J11.6, 8.1), 3.97 (1H, dt, J 11.7, 3.9), 2.65 (1H, dd, J8.5, 3.8). ¹³C NMR (100 MHz, CDCl₃) δ 168.6, 134.2, 132.0, 131.8, 123.5, 118.9, 63.0, 56.0.

### Example 20

The enantioselective functionalization of prochiral heterocycles is a vast field with scores of possible methodologies. In Pd-AAA, the three predominant methods involve either the direct allylation of a prochiral heterocycle, the allylation of the corresponding enol silane, or the allylation via a decarboxylative pathway. In this example, the product of each of these methods was improved upon greatly by using chiral precatalyst embodiments according to the present disclosure. The direct Pd-AAA approach and the Pd-DAAA both suffered when lowering the catalyst loading from 10 to 2 mol % in the case of Pd₂dba₃, resulting in extended reaction times and little to no reactivity. Interestingly, running the reaction of the enol silane using 2 mol % of Pd resulted in full conversion and good yields after only 15 minutes, however the reactivity was lost when the Pd:L ratio was reduced to 1:1. This was circumvented by using 2 mol % of ^{Ph}DACH-Pd-MAH under otherwise identical conditions. This 5-fold lower catalyst loading when compared to Pd₂dba₃ has a profound impact on the practicality, the cost, and the sustainability of the process. Results are summarized in Tables 15, 16, and 17.

3-Bromo-2,5-dihydrofuran-2-one: To a solution of 2(5H)-furanone (2.00 mL, 28.2 mmol, 1.00 equiv.) in benzene (30.0 mL), was added bromine (1.70 mL, 31.0 mmol, 1.10 equiv.). The solution was stirred for 24 hours at room temperature and was then cooled to 0° C. Pyridine (6.80 mL, 84.6 mmol, 3.00 equiv.) was added dropwise and the mixture was stirred at 0° C. for 2 hours and then at room temperature for another 2 hours. The reaction mixture was cooled to 0° C., filtered on a silica gel pad to remove the pyridine salts and rinsed with cold diethyl ether. The filtrate was concentrated under vacuum and the crude was eventually purified by flash column chromatography over silica gel to recover the title compound (3.76 g, 23.1 mmol, 82%) as a white/yellowish solid. ¹H NMR (400 MHz, CDCl₃) δ 7.64 (t, J=1.9 Hz, 1H, H₃), 4.86 (d, J=1.9 Hz, 2H, H₂). ¹3C NMR (101 MHz, CDCl₃) δ 169.0, 149.4, 113.1, 71.7.

3-Phenyl-2,5-dihydrofuran-2-one: To a solution of 3-Bromo-2,5-dihydrofuran-2-one (163 mg, 1 mmol, 1.00 equiv.) in benzene (3.00 mL), followed by phenyl boronic acid (182 mg, 1.5 mmol, 1.50 equiv.), Pd(PPh₃)₄ (57 mg, 5 mol %) and a solution of Na₂CO₃ (2.00 equiv.) in distilled H₂O (1.00 mL). The reaction mixture was heated in a sealed vessel at 100° C. for 30 min under microwave irradiation (300 W). Brine was then added and the aqueous phase was extracted with ethyl acetate. The combined organics were dried over MgSO₄, filtered and the solvent removed under reduced pressure. The residue was purified by flash column chromatography over silica gel (n-Hexane/Ethyl acetate, 75:25) to recover the title compound (88.0 mg, 0.55 mmol, 55% yield) as a slightly yellow solid. ¹H NMR (400 MHz, CDCl₃) δ 7.86 (m, 1H, HAr), 7.84 (m, 1H, HAr), 7.65 (t, J=2.0 Hz, 1H, H₃), 7.46-7.36 (m, 3H, HAr), 4.92 (d, J=2.0 Hz, 2H, H₂). ¹³C NMR (101 MHz, CDCl₃) δ 172.3, 144.5, 131.6, 129.5, 129.3, 128.7, 127.0, 69.6.

3-Phenylfuran-2-yl prop-2-en-1-ylcarbonate:¹H NMR (400 MHz, CDCl₃) δ 7.52-7.48 (m, 2H, HAr), 7.42-7.37 (m, 2H, HAr), 7.28 (m, 1H, HAr), 7.18 (d, J=2.3 Hz, 1H, H₂), 6.68 (d, J=2.3 Hz, 1H, H₃), 5.97 (ddt, J=17.3, 10.5, 131 5.8 Hz, 1H, H₇), 5.43 (dq, J=17.3, 1.3 Hz, 1H, H₈), 5.35 (dq, J=10.5, 1.1 Hz, 1H, H₈), 4.77 (dtapp, J=5.9, 1.3 Hz, 2H, H₆). ¹³C NMR (101 MHz, CDCl₃) δ 151.7, 146.3, 136.5, 130.7, 130.4, 128.8, 127.0, 126.4, 120.1, 111.0, 108.1, 70.2.

tert-butyldimethyl((3-phenylfuran-2-yl)oxy)silane: To a solution of 3-Phenyl-2,5-dihydrofuran-2-one (0.25 mmol, 1.00 equiv.) in anhydrous CH₂Cl₂ (2.50 mL), was added triethylamine (3.00 equiv.) and TBSOTf (2.00 equiv.). The reaction mixture was heated at reflux overnight. After cooling to room temperature, the solvent was removed under reduced pressure. The crude residue was eventually purified by suction filtration over silica gel (n-Hexane/triethylamine, 99:1). After evaporation, an internal standard (DME) was used to quantify the effective quantity of the desired protected product. The crude was engaged directly into the next step, due to its instability.

(3S)-3-Phenyl-3-(prop-2-en-1-yl)-2,3-dihydrofuran-2-one: prepared using the various methods outlined below. ¹H NMR (400 MHz, CDCl₃) δ 7.51-7.45 (m, 2H, H₉), 7.40-7.36 (m, 2H, H₁₀), 7.31 (3 m, 1H, H₁₁), 6.93 (d, J=3.5 Hz, 1H, H₂), 5.89 (d, J=3.5 Hz, 1H, H₃), 5.61 (m, 1H, H₆), 5.15 (2 m, 1H, H₇), 5.12 (2 m, 1H, H₇), 2.88-2.76 (m, 2H, H₅). 13C NMR (101 MHz, CDCl₃) δ 178.5 (Cq, C₁), 141.8 (CH, C₂), 137.9 (Cq, C₈), 131.8 (CH, C₆), 128.8 (CH, 2C, C₉), 127.9 (CH, C₁₁), 126.4 (CH, 2C, C₁₀), 119.8 (CH₂, C₇), 113.2 (CH, C₃), 54.5 (Cq, C₄), 43.3 (CH₂, C₅). HPLC: Chiralpak IB column, T=30° C., n-Hexane/i-PrOH=98:2, flow rate=1 mL/min, A=230 nm, tR=7.149 min (minor) and tR=8.001 min (major).

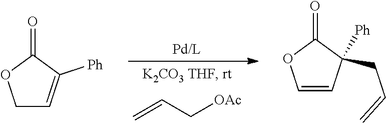

TABLE 15

| | Pd | Pd | L | | | Yield | ee |
|---|---|---|---|---|---|---|---|
| Entry | source | (mol %) | (mol %) | Pd:L | Time | % | % |
| 1 | Pd₂dba₃ | 10 | 12 | 1:1.2 | 24 h | 86 | 83 |
| 2 | Pd₂dba₃ | 2 | 2.5 | 1:1.25 | 3 d | 12 | 78 |
| 3 | Pd₂dba₃ | 2 | 2 | 1:1 | 3 d | 10 | 75 |
| 4 | ^{Ph}DACH-Pd-MAH | 2 | — | 1:1 | 3 d | 65 | 82 |

Either: A mixture of Pd₂dba₃ (indicated mol %) and SS-DACH-Phenyl (indicated mol %) in THF (0.5 mL) was stirred for 30 mins at room temperature, Or: A solution of ^{Ph}DACH-Pd-MAH (3.6 mg, 2 mol %) in THF (0.5 mL) was prepared. The catalyst solution was then added to a mixture of 3-phenylfuran-2(5H)-one (32 mg, 0.2 mmol) and K₂CO₃ (41.4 mg, 0.3 mmol, 1.5 equiv) in THF (1.5 mL), followed by allyl acetate (30 mg, 32 μL, 0.3 mmol, 1.5 equivs). The reaction was stirred at room temperature for the indicated time.

The reaction was filtered through Celite and the solvent removed under reduced pressure. The residue was purified by flash column chromatography over silica gel (n-Hexane/Et₂O, 70:30) to recover (S)-3-allyl-3-phenylfuran-2(3H)-one (indicated yield) as a colorless oil.

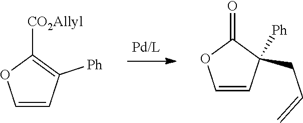

TABLE 16

| | Pd | Pd | L | | | Yield | ee |
|---|---|---|---|---|---|---|---|
| Entry | source | (mol %) | (mol %) | Pd:L | Time | % | % |
| 1 | Pd₂dba₃ | 10 | 12 | 1:1.2 | 1 h | 80 | 82 |
| 2 | Pd₂dba₃ | 2 | 2.5 | 1:1.25 | 3 d | <5 | — |
| 3 | Pd₂dba₃ | 2 | 2 | 1:1 | 3 d | <5 | — |
| 4 | ^{Ph}DACH-Pd-MAH | 2 | — | 1:1 | 4 h | 75 | 82 |

Either: A mixture of Pd₂dba₃ (indicated mol %) and SS-DACH-Phenyl (indicated mol %) in THF (0.5 mL) was stirred for 30 mins at room temperature, Or: A solution of ^{Ph}DACH-Pd-MAH (2.7 mg, 2 mol %) in THF (0.5 mL) was prepared. The catalyst solution was then added to a mixture of allyl 3-phenylfuran-2-carboxylate (35 mg, 0.15 mmol) in THF (1.5 mL). The reaction was stirred at room temperature for the indicated time. The reaction was filtered through Celite and the solvent removed under reduced pressure. The residue was purified by flash column chromatography over silica gel (n-Hexane/Et₂O, 70:30) to recover (S)-3-allyl-3-phenylfuran-2(3H)-one (indicated yield) as a colorless oil.

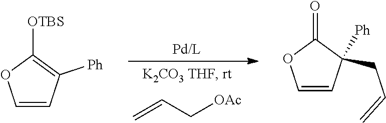

TABLE 17

| | Pd | Pd | L | | | Yield | ee |
|---|---|---|---|---|---|---|---|
| Entry | source | (mol %) | (mol %) | Pd:L | Time | % | % |
| 1 | Pd₂dba₃ | 10 | 12 | 1:1.2 | 1 h | 85 | 75 |
| 2 | Pd₂dba₃ | 2 | 2.5 | 1:1.25 | 15 min | 70 | 78 |
| 3 | Pd₂dba₃ | 2 | 2 | 1:1 | 2 d | <5 | — |
| 4 | ^{Ph}DACH-Pd-MAH | 2 | — | 1:1 | 15 min | 67 | 75 |

Either: A mixture of Pd₂dba₃ (indicated mol %) and SS-DACH-Phenyl (indicated mol %) in THE (0.5 mL) was stirred for 30 mins at room temperature, Or: A solution of ^{Ph}DACH-Pd-MAH (3.6 mg, 2 mol %) in THE (0.5 mL) was prepared. The catalyst solution was then added to a mixture of tert-butyldimethyl((3-phenylfuran-2-yl)oxy)silane (55 mg, 0.2 mmol) and K₂CO₃ (41.4 mg, 0.3 mmol, 1.5 equiv) in THE (1.5 mL), followed by allyl acetate (30 mg, 32 μL, 0.3 mmol, 1.5 equivs). The reaction was stirred at room temperature for the indicated time. The reaction was filtered through Celite and the solvent removed under reduced pressure. The residue was purified by flash column chromatography over silica gel (n-Hexane/Ethyl acetate, 70:30) to recover (S)-3-allyl-3-phenylfuran-2(3H)-one (indicated yield) as a colorless oil.

### Example 21

The precatalyst embodiments of the present disclosure notably evade the need of an additional catalyst formation step. This feature lends to the precatalysts' use in high-throughput experimentation (HTE), a technique which has revolutionized reaction optimization. Indeed, bench stable single-component precatalysts render the procedure operationally simple and eliminate routine requirement of a glovebox.

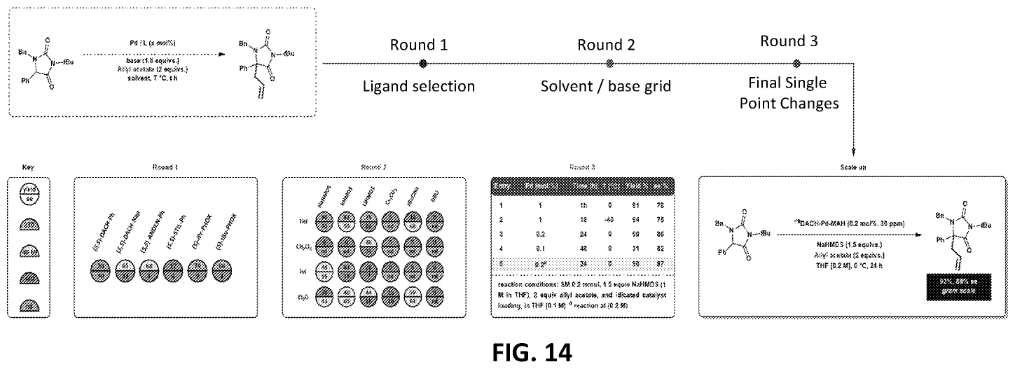

The complexed Pd-MAH catalysts have been showed to be stable in solution in THE under air. In this example, the practicality of disclosed precatalysts in the rapid HTE optimization of the Pd-AAA of hydantoins, a heterocycle among the most common heterocycles in FDA approved drugs with a worrying lack of methods for asymmetric functionalization. The methods used in this example are summarized in FIG. 14 and results provided in Table 18 (Round 1), Table 19 (Round 2), and Table 20 (Round 3).

Round 1 of screening employed ^{DMP}DAB-Pd-MAH (2 mol %) to screen ligands for the process (Pd/L, 1:1.5) in the presence of base and solvent. notably, only DACH-Phenyl gave good selectivity with all other ligands providing very poor selectivity.

Round 2 screening utilized chiral Pd(0) precursor complex ^{Ph}DACH-Pd-MAH (2 mol %) in a solvent base grid to rapidly optimize the conditions. Stock solutions of the catalysts, ligands, and starting materials were all prepared under air. With the single component ^{Ph}DACH-Pd-MAH, the reaction proceeded at an extremely low catalyst loading (0.2 mol % or 30 ppm) with a concomitant increase in enantioselectivity. Here, the concentration could be easily modulated (0.2 M) to get an effective transformation while maintaining a stoichiometry in Pd. In some examples, the following conditions were used in the gram scale alkylation with no loss in activity or selectivity and the product was isolated in 93% yield and 88% ee: ^{Ph}DACH-Pd-MAH 0.2 mol %, NaHMDS 1.5 equiv., allyl acetate 2 equiv., THE [0.2M].

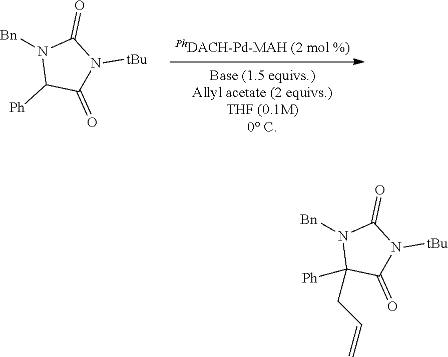 [SMILES: CC(C)(C)N1C(=O)C(c2ccccc2)N(Cc2ccccc2)C1=O ; C=CCC1(c2ccccc2)C(=O)N(C(C)(C)C)C(=O)N1Cc1ccccc1]

1-benzyl-3-(tert-butyl)-5-phenylimidazolidine-2,4-dione. To a solution of methyl 2-benzamido-2-phenylacetate (3.5 g, 13.7 mmol) in THE (30 mL) was added NEt₃ (3.1 ml, 22 mmol, 1.6 equiv.) and tBuNCO (2.03 g, 2.3 mL, 20.5 mmol, 1.5 equiv.). The solution was heated at 65° C. for 18 hours before being allowed to cool to room temperature. The solvent was removed under reduced pressure. The residue was reconstituted in THE (45 mL), and tBuOK (2.3 g, 20.5 mmol, 1.5 equiv.) was added. The reaction was stirred for 1 hour at room temperature. H₂O (100 ml) was added and the mixture extracted with CH₂Cl₂ (3×100 ml). The combined organics were dried over MgSO₄, filtered and the solvent removed under reduced pressure. The residue was purified by flash column chromatography over silica gel eluting with EtOAc: Hex 1:9 to recover the title compound (2.5 g, 7.75 mmol, 57%) as a colorless solid, which could be further purified by recrystallization from hexane (˜5 mL/g). m.p 88-90° C., Rf: 0.5 (3:7 EtOAc:Hex, UV active). ¹H NMR (400 MHz, CDCl₃) δ 7.44-7.37 (3H, m), 7.34-7.28 (3H, m), 7.19-7.14 (2H, m), 7.14-7.09 (2H, m), 5.12 (1H, d, J14.9), 4.49 (1H, s), 3.69 (1H, d, J14.9), 1.65 (9H, s).¹³C NMR (100 MHz, CDCl₃) δ 172.2, 157.5, 135.8, 133.6, 129.4, 129.3, 129.0, 128.6, 128.1, 127.6, 62.5, 58.4, 44.5, 28.9. IR (neat) v max: 3005, 2986, 2938, 1763, 1700, 1411, 1189, 1133, 707. HRMS (ESI+): m/z calcd for C₂₀H₂₂N₂O₂⁺[M⁺H]⁺: requires 323.1760, found 323.1728

All HTE reaction were performed in a Radleys GreenHouse Parallel Synthesiser under N₂. Stock solutions of ^{DMP}DAB-Pd-MAH (5 mg/mL), chiral ligand (concentration set so that 100 μL equates to 3 mol % of the ligand; e.g. (S,S)-^{Ph}DACH was 21 mg/mL, (S,S)-^{NAPH}DACH was 24 mg/mL, (S,S)-^{Ph}STIL was 24 mg/mL, (S,S)-^{Ph}ANDEN was 25 mg/mL, ^{iPr}PHOX was 11 mg/mL, ^{tBu}PHOX was 11 mg/mL), allyl acetate (470 mg/mL) and 1-benzyl-3-(tert-butyl)-5-phenylimidazolidine-2,4-dione (322 mg/mL), and ^{Ph}DACH-Pd-MAH (4.5 mg/mL) were all prepared in THE and were handled under air.

Round 1—The ^{DMP}DAB-Pd-MAH (200 μL, 1 mg, 2 mol %) followed by appropriate ligand solution (100 μL, 3 mol %) and allowed to stir for ˜5 mins before the addition of SM (500 μL, 0.1 mmol). NaHMDS (150 μL, 1 M, 1.5 equivs. commercial solution) was then added, and the mixture stirred for 1 hour. Finally, the solution of allyl acetate (50 μL, 2 equivs.) was added, and the reactions were stirred for 18 hours at room temperature. MeOH (1 mL) was added, and the solvent removed by blow down. Yields were obtained by H¹ NMR using DME as an internal standard. Successful reactions (>10% yield) were purified by a short preparative TLC and ee was determined by chiral HPLC.

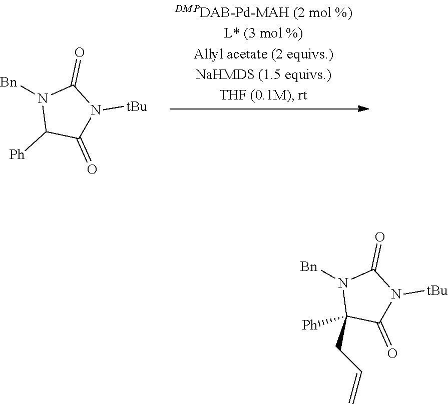

TABLE 18

| Entry | Ligand | Yield % | ee % |
|---|---|---|---|
| 1 | (S,S)-^{Ph}DACH | 70 | 70 |
| 2 | (S,S)-^{NAP}DACH | 65 | 32 |
| 3 | (S,S)-^{Ph}ANDEN | 68 | 4 |
| 4 | (S,S)-^{Ph}STIL | 17 | 9 |
| 5 | (S)-^{iPr}PHOX | 79 | 9 |

Round 2—Stock solutions were made in respective solvents: 1-benzyl-3-(tert-butyl)-5-phenylimidazolidine-2,4-dione (107.2 mg/mL); ^{Ph}DACH-Pd-MAH (4.5 mg/mL); Allyl acetate (1466 μL in 10 mL); base stock solutions were either used as commercial 1 M solutions (LHMDS, NaHMDS, KHMDS) or made up to respective concentrations. If reagent proved insoluble in respective solvent, then a solution was made in a different solvent, the respective amount added to a well and the solvent removed by blowdown then reduced pressure before being reconstituted in the required solvent. Representative procedure: 300 μL of SM stock solution was added to a well followed by 150 μL of base solution (1 M) and stirred at room temperature for 1 hour. ^{Ph}DACH-Pd-MAH stock solution (400 μL) was added followed by allyl acetate stock solution (150 μL). The reactions were stirred for 18 hours at room temperature. MeOH (1 mL) was added, and the solvent removed by blow down. Yields were obtained by H¹ NMR using DME as an internal standard. Successful reactions (>10% yield) were purified by a short preparative TLC and ee was determined by chiral HPLC.

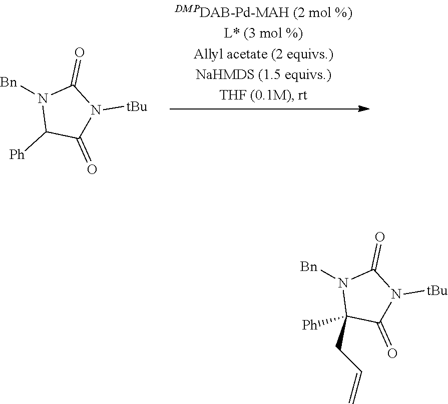

TABLE 19

| Entry | Ligand | Yield % | ee % |
|---|---|---|---|
| 1 | (S,S)-^{Ph}DACH | 70 | 70 |
| 2 | (S,S)-^{NAP}DACH | 65 | 32 |
| 3 | (S,S)-^{Ph}ANDEN | 68 | 4 |
| 4 | (S,S)-^{Ph}STIL | 17 | 9 |
| 5 | (S)-^{iPr}PHOX | 79 | 9 |

Representative procedure for 0.2 mol % Pd: To a solution of 1-benzyl-3-(tert-butyl)-5-phenylimidazolidine-2,4-dione (64 mg, 0.2 mmol, 1 equiv.) in THF (340 μL) at 0° C., was added NaHMDS (1 M in THF, 300 μL, 1.5 equivs) and the solution stirred for 30 min. ^{Ph}DACH-Pd-MAH (1 mg/mL in THF, 360 μL, 0.4 μmol, 0.2 mol %) was then added, followed by allyl acetate (43 μL, 0.4 mmol, 2 equivs.). The reaction was stirred at 0° C. for 24 hours. H₂O (10 mL) was added and then extracted with CH₂Cl₂ (3×10 mL). The combined organics were dried over MgSO₄, filtered and the solvent removed under reduced pressure. The residue was purified by flash column chromatography over silica gel eluting with Hexane and EtOAc (9:1) to recover the title compound (65 mg, 0.18 mmol, 90%) as a colorless oil.

5-allyl-1-benzyl-3-(tert-butyl)-5-phenylimidazolidine-2,4-dione: To a solution of 1-benzyl-3-(tert-butyl)-5-phenylimidazolidine-2,4-dione (1.29 g, 4 mmol, 1 equiv.) in THE (14 mL) at 0° C. was added NaHMDS (1M in THF, 6 mL, 6 mmol, 1.5 equiv.) and the solution stirred for 1 hour. ^{Ph}DACH-Pd-MAH (7.2 mg, 0.008 mmol, 0.2 mol %) was then added as a solid followed by Allyl acetate (860 μL, 8 mmol, 2 equiv.). The reaction was stirred at 0° C. for 24 hours at which time the reaction was deemed complete by TLC. H₂O (50 mL) was added and the mixture extracted with CH₂Cl₂ (3×50 mL). The combined organics were then washed with 10% w/w aqueous Citric acid (2×50 mL) and brine (50 mL). the organic phase was dried over MgSO₄, filtered, and the solvent removed under reduced pressure. The residue was purified by silica gel column chromatography eluting with Hexane and EtOAc (100:0 to 80:20) to recover the title compound (1.35 g, 3.7 mmol, 93%) as a colorless oil. Rf: 0.55 (3:7 EtOAc:Hex, Uv active)¹H NMR (400 MHz, CDCl₃) δ 7.30-7.24 (3H, m), 7.16-7.09 (7H, m), 5.27-5.15 (1H, m), 4.97-4.81 (2H, m), 4.60 (1H, d, J 15.1), 3.82 (1H, d, J15.1), 2.96 (1H, dd, J13.7, 7.7), 2.59 (1H, dd, J13.7, 6.4), 1.56 (9z H, s). ¹3C NMR (100 MHz, CDCl₃) δ 174.7, 158.1, 137.4, 137.0, 130.2, 129.4, 129.2, 128.8, 128.5, 127.6, 126.6, 121.0, 69.6, 58.4, 44.7, 37.1, 28.9. IR (neat) v max: 2973, 2932, 1767, 1704, 1398, 1132, 1034, 761, 696. HRMS (ESI+): m/z calcd for C₂₃H₂₇N₂O₂+[M⁺H]⁺: requires 363.2072, found 363.2080. HPLC: Diacel chiralpac IC, hexane/IPA: 90/10, 1 mL/min, 35° C., A=230 nm, tR(maj) 6.06 min, tR(min) 6.69 min.

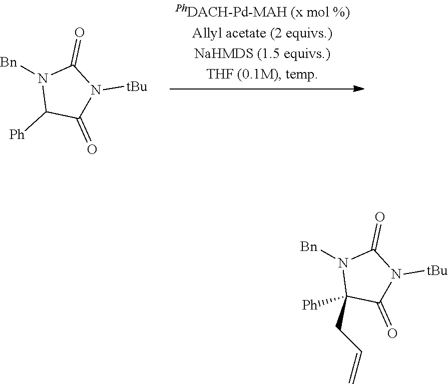

TABLE 20

| Entry | Pd (mol %) | Time (h) | T (° C.) | Yield % | ee % |
|---|---|---|---|---|---|
| 1 | 1 | 1 | 0 | 91 | 76 |
| 2 | 1 | 18 | −40 | 94 | 75 |
| 3 | 0.2 | 24 | 0 | 90 | 86 |
| 4 | 0.1 | 48 | 0 | 31 | 82 |
| 5^{a} | 0.2 | 24 | 0 | 90 | 87 |

Reaction conditions: SM 0.2 mmol, 1.5 equiv NaHMDS (1M in THF), 2 equiv allyl acetate, and indicated catalyst loading and temperature, in THF (0.1M).
^{a}reaction run at 0.2M concentration.

In view of the many possible embodiments to which the principles of the present disclosure may be applied, it should be recognized that the illustrated embodiments are only preferred examples and should not be taken as limiting the scope of the present disclosure. Rather, the scope is defined by the following claims. We therefore claim as our invention all that comes within the scope and spirit of these claims.

1. A chiral precatalyst having a structure according to Formula I 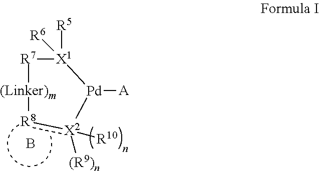
wherein:
the Pd atom has an oxidation state of 0;
the A group is a compound comprising at least one olefin moiety capable of coordinating with Pd(0), provided that the A group is not (1 E,4E)-1,5-diphenylpenta-1,4-dien-3-one or methyl (E)-2-acetoxy-3,5-diphenylpent-4-enoate;
each of X¹ and X² independently are selected from phosphorus or nitrogen, provided that if one of X¹ or X² is nitrogen, then the other of X¹ or X² is phosphorus and is not nitrogen;
each of R⁵ and R⁶ independently is selected from aliphatic, heteroaliphatic, haloaliphatic, haloheteroaliphatic, aromatic, or an organic functional group;
R⁷ is an aromatic group, an aliphatic group, a haloaliphatic group, a haloheteroaliphatic group, or a heteroaliphatic group;
the linker, if present, comprises a chiral heteroaliphatic group having a formula 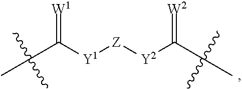
wherein Z comprises an aliphatic group, an aromatic group, or an organic functional group, wherein the aliphatic, aromatic, or organic functional group comprises a chiral substituent and/or an asymmetric center; and each of W¹, W², Y¹, and Y² independently are selected from oxygen, sulfur, or NR wherein R is hydrogen, aliphatic, or aromatic;
each of R⁹ and R¹⁰, if present, independently is selected from aliphatic, heteroaliphatic, haloaliphatic, haloheteroaliphatic, aromatic, or an organic functional group;
each n is 1 or each n is 0;
m is 1 or 0; and
(i) if each n is 1, then R⁸ is an aromatic group, an aliphatic group, a haloaliphatic group, a haloheteroaliphatic group, or a heteroaliphatic group and is bound to X² via a single bond and no ring B is formed; or
(ii) if each n is 0, then R⁸ is a carbon atom that forms the ring B with X² and is bound to X² via a double bond, wherein the ring B is an aromatic or heterocyclic ring comprising a chiral substituent or an asymmetric center; and
provided that for Formula I, the compound is not or is other than

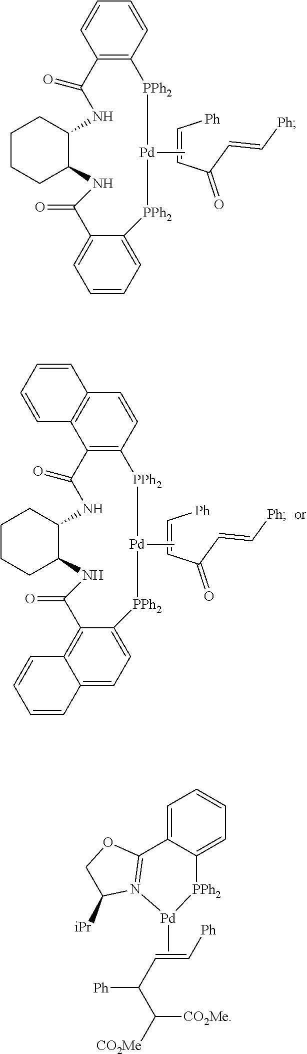

2. The chiral precatalyst of claim 1, wherein the A group has a Formula II 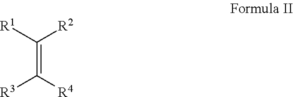
wherein:
each of R¹, R², R³, and R⁴ independently is (i) selected from hydrogen, aliphatic, aromatic, or an electron-withdrawing group provided that at least one of R¹, R², R³, or R⁴ is other than hydrogen; or (ii) R¹ and R³, or R² and R⁴ join together to provide a cyclic group and the remaining R¹ and R³ groups, or R² and R⁴ groups are hydrogen.

3. The chiral precatalyst of claim 1, wherein the A group has a structure selected from

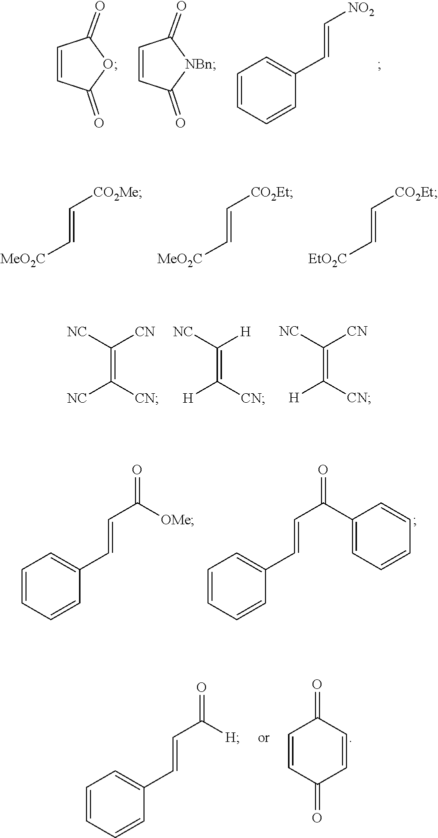

4. The chiral precatalyst of claim 2 having a structure according to Formula III

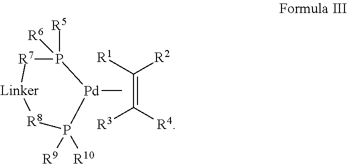

5. The chiral precatalyst of claim 1, wherein Z is selected from a cyclohexyl group, a diarylethane group, or a 9,10-dihydro-9,10-ethanoanthracene group; each of Y¹ and Y² is NH; and each of W¹ and W² is oxygen.

6. The chiral precatalyst of claim 1, wherein each of R⁵, R⁶, R⁹, and R¹⁰ is phenyl.

7. The chiral precatalyst of claim 4, having a structure according to any one of Formulas IIIA-IIIC 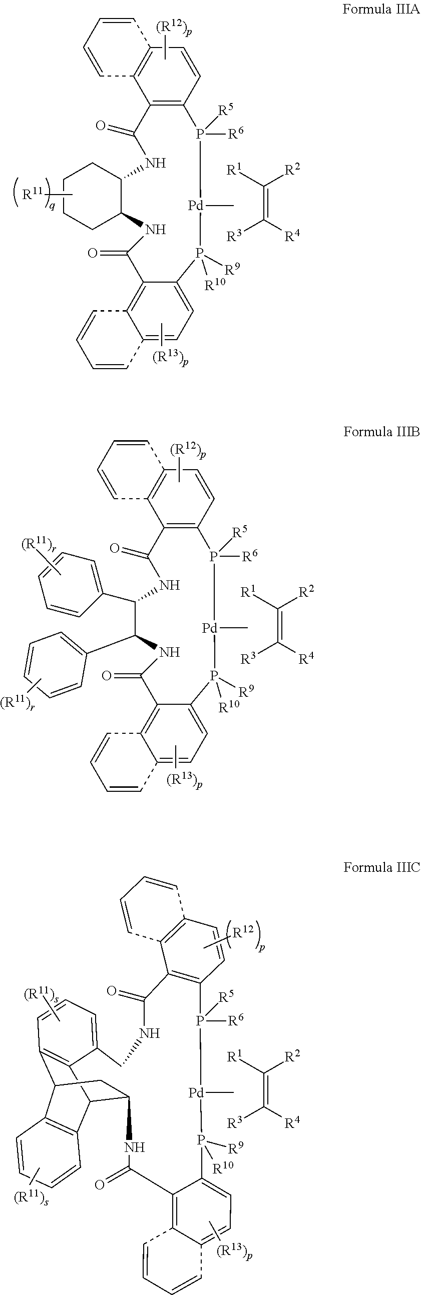
wherein:
each of R¹, R², R³, and R⁴ independently is (i) selected from hydrogen, aliphatic, aromatic, or an electron-withdrawing group provided that at least one of R¹, R², R³, or R⁴ is other than hydrogen; or (ii) R¹ and R³, or R² and R⁴ join together to provide a cyclic group and the remaining R¹ and R³ groups, or R² and R⁴ groups are hydrogen;
each R¹¹ independently is selected from aliphatic, heteroaliphatic, haloaliphatic, aromatic, or an organic functional group;
each of R¹² and R¹³ independently, for each occurrence, is selected from aliphatic, heteroaliphatic, aromatic, haloaliphatic, or an organic functional group;
p is an integer selected from 0 to 6;
q is an integer ranging from 0 to 10;
each r independently is an integer selected from 0 to 5; and
each s independently is an integer selected from 0 to 4.

8. The chiral precatalyst of claim 7, wherein each R¹¹ independently is selected from alkyl, alkenyl, alkynyl, heteroalkyl, heteroalkenyl, heteroalkynyl, aryl, heteroaryl, nitro, hydroxyl, amine, halogen, cyano, thiol, and haloalkyl; and wherein each of R¹² and R¹³ independently, for each occurrence is selected from aliphatic, heteroaliphatic, aromatic, nitro, hydroxyl, amine, halogen, cyano, thiol, and haloalkyl.

9. The chiral precatalyst of claim 1 having a structure according to Formula IV 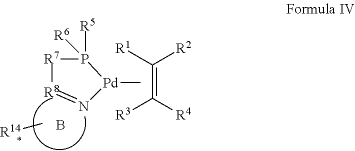
wherein:
each of R¹, R², R³, and R⁴ independently is (i) selected from hydrogen, aliphatic, aromatic, or an electron-withdrawing group provided that at least one of R¹, R², R³, or R⁴ is other than hydrogen; or (ii) R¹ and R³, or R² and R⁴ join together to provide a cyclic group and the remaining R¹ and R³ groups, or R² and R⁴ groups are hydrogen;
R¹⁴ is selected from aliphatic, heteroaliphatic, aromatic, or an organic functional group; and
the * symbol indicates that R¹⁴ is chiral or is attached to an asymmetric carbon atom of ring B.

10. The chiral precatalyst of claim 9, wherein ring B is a five-membered or six-membered heterocycle.

11. The chiral precatalyst of claim 9, wherein R¹⁴ is an alkyl group that is attached to an asymmetric carbon atom of ring B.

12. The chiral precatalyst of claim 1, having a structure according to Formula IVA or IVB 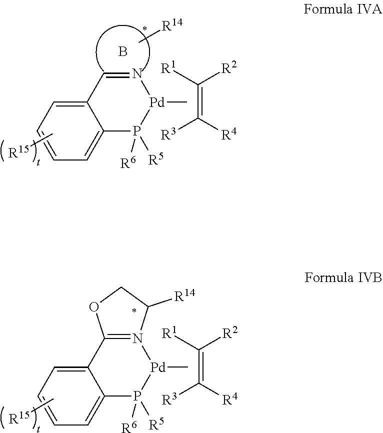
wherein:
each of R¹, R², R³, and R⁴ independently is (i) selected from hydrogen, aliphatic, aromatic, or an electron-withdrawing group provided that at least one of R¹, R², R³, or R⁴ is other than hydrogen; or (ii) R¹ and R³, or R² and R⁴ join together to provide a cyclic group and the remaining R¹ and R³ groups, or R² and R⁴ groups are hydrogen:
R¹⁴ is selected from aliphatic, heteroaliphatic, aromatic, or an organic functional group:
each R¹⁵ independently is selected from aliphatic, heteroaliphatic, haloaliphatic, aromatic, or an organic functional group;
the * symbol indicates that R¹⁴ is chiral or is attached to an asymmetric carbon atom of ring B; and
t is an integer selected from 0 to 4.

13. The chiral precatalyst of claim 12, wherein each R¹⁵ independently is selected from alkyl, alkenyl, alkynyl, heteroalkyl, heteroalkenyl, heteroalkynyl, aryl, heteroaryl, nitro, hydroxyl, amine, halogen, cyano, thiol, and haloalkyl.

14. The chiral precatalyst of claim 1, wherein the chiral precatalyst is selected from:

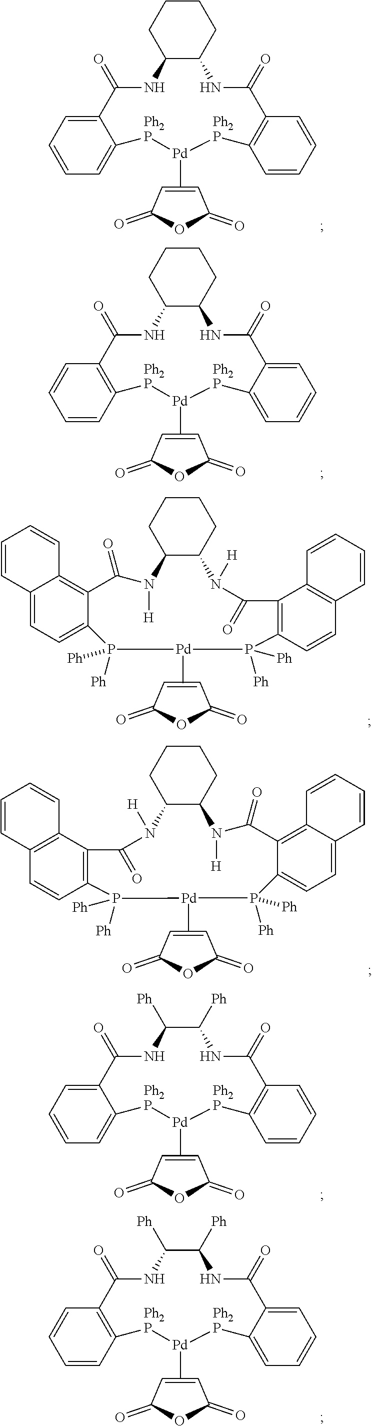

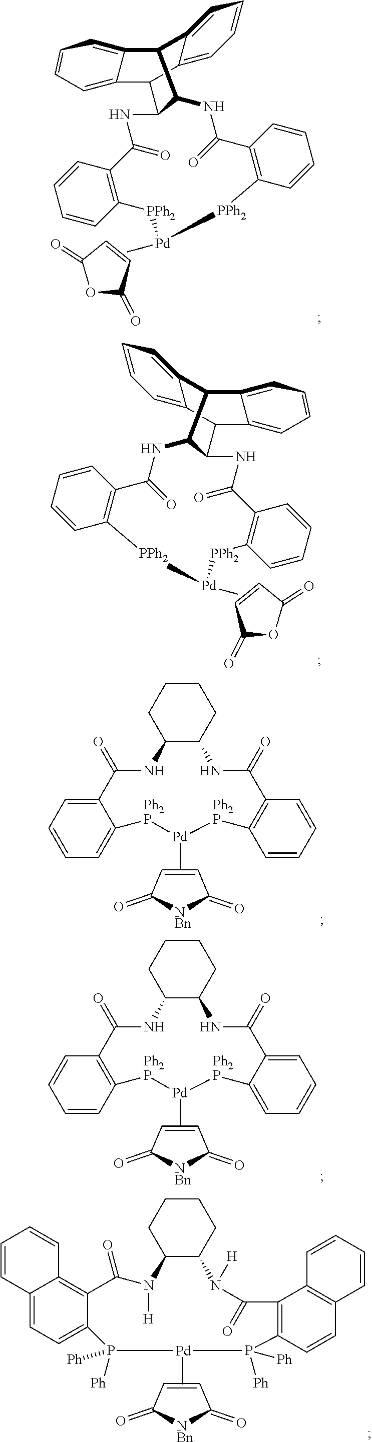

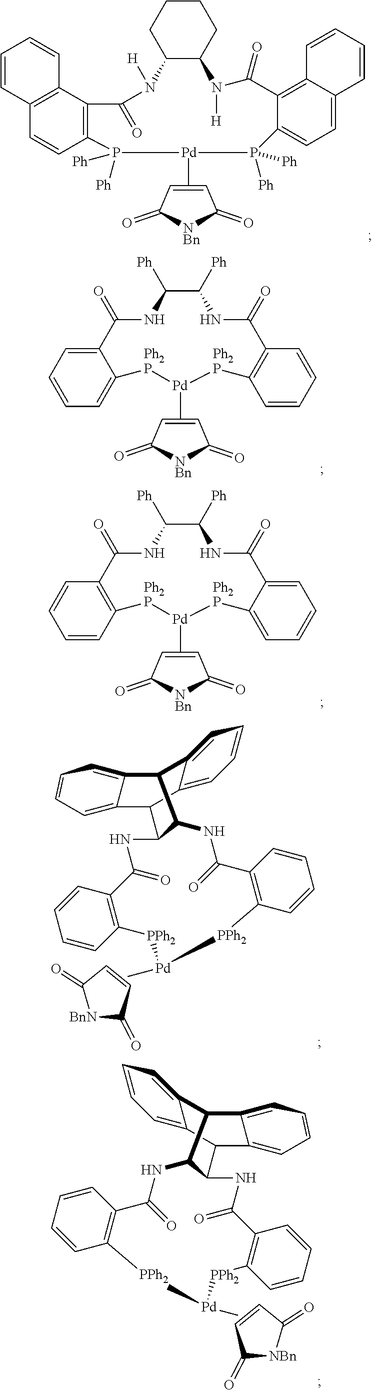

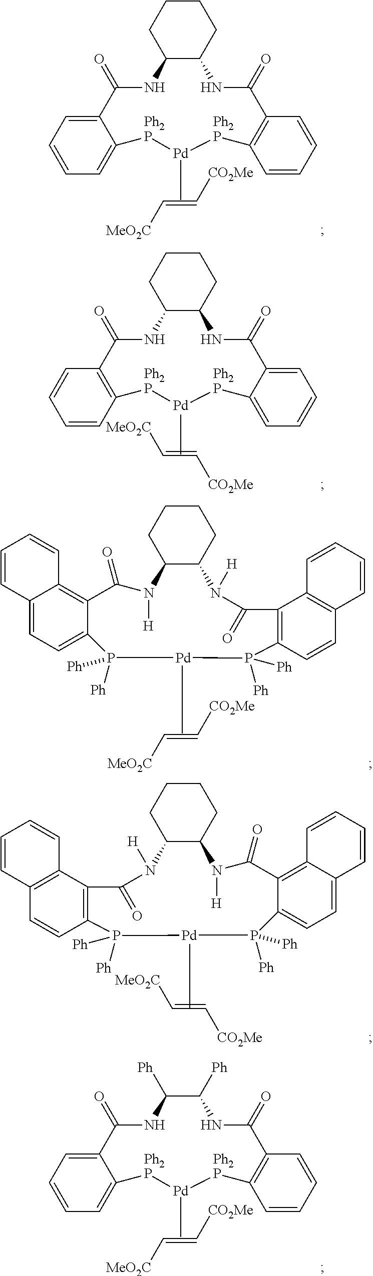

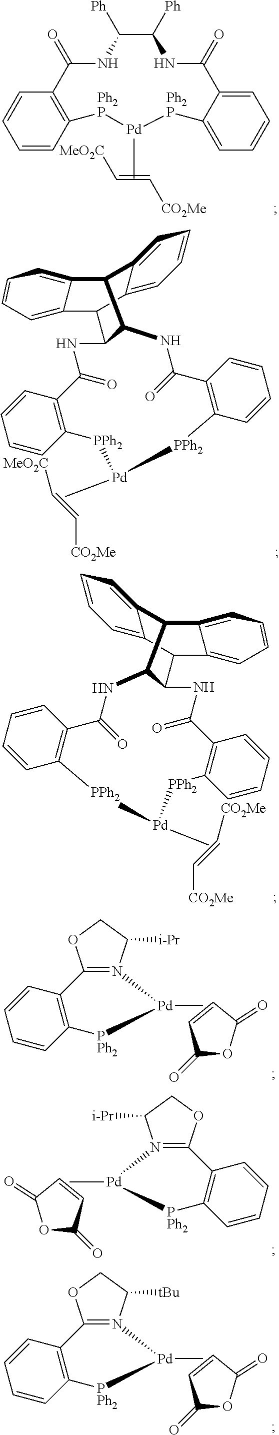

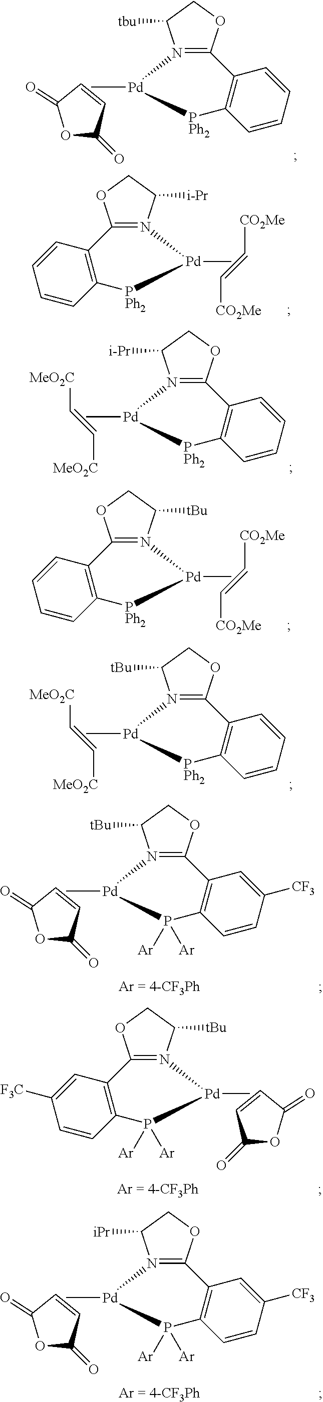

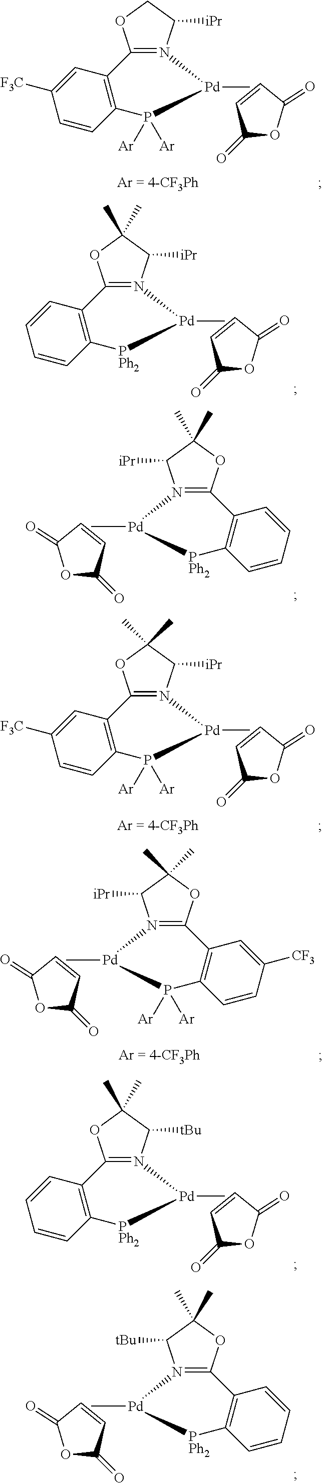

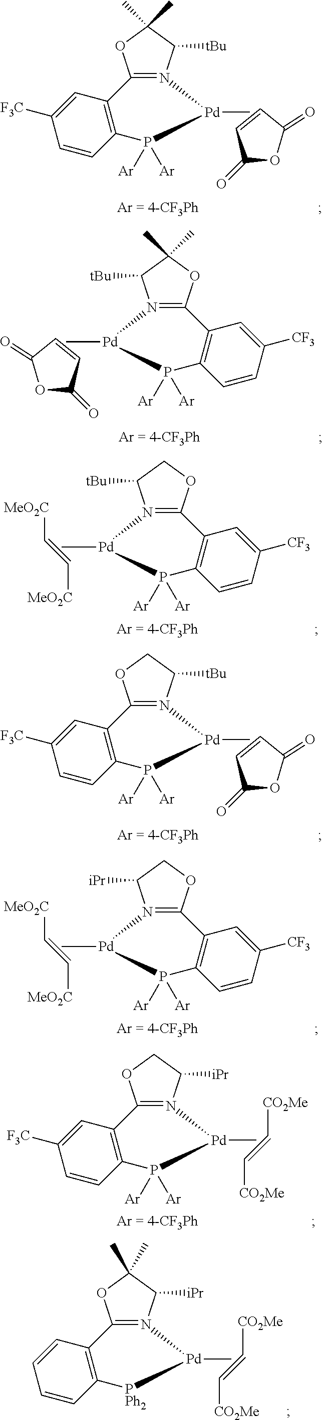

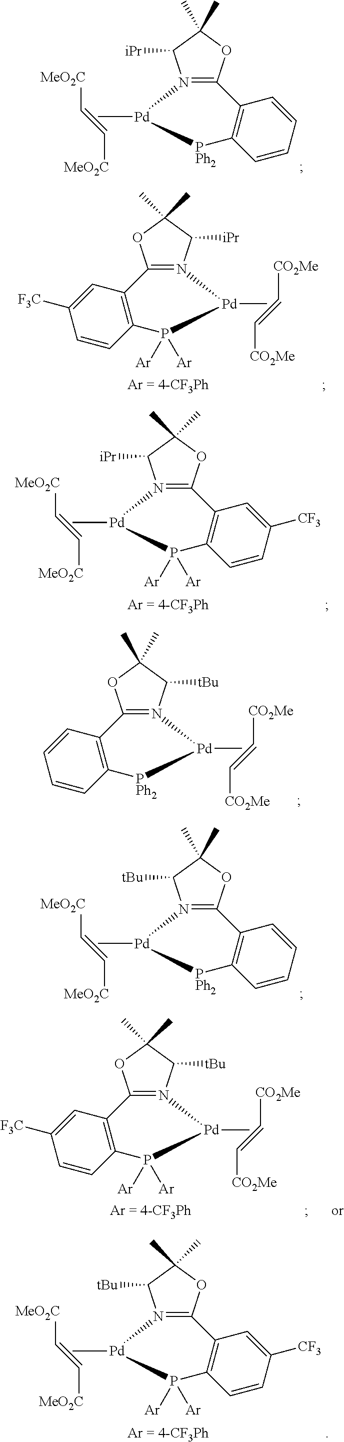

15. A method of making the chiral precatalyst of claim 1, comprising exposing a Pd(0) precursor complex having a structure according to Formula A to a donor atom-containing ligand compound having a structure according to Formula V:
wherein Formula A is 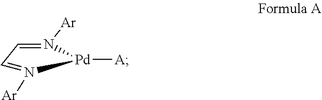
wherein A is as recited in claim 1 and Ar is aromatic; and
Formula V is

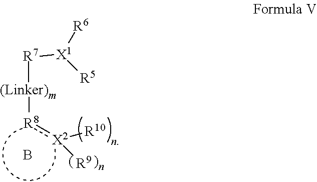

16. The method of claim 15, wherein the Pd(0) precursor complex is

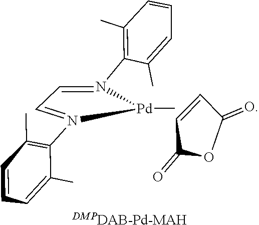

17. The method of claim 15, wherein the donor atom-containing ligand compound has a structure according to Formula VI or Formula VII

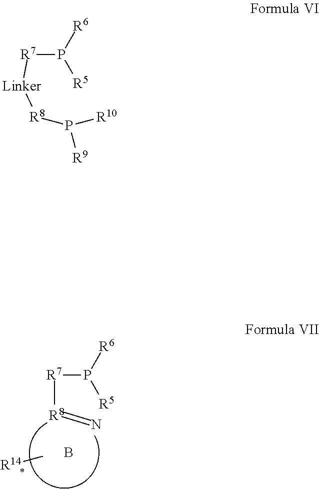

18. The method of claim 15, wherein the donor atom-containing ligand compound has a structure according to Formula VIA, VIB, VIC, or VIIA 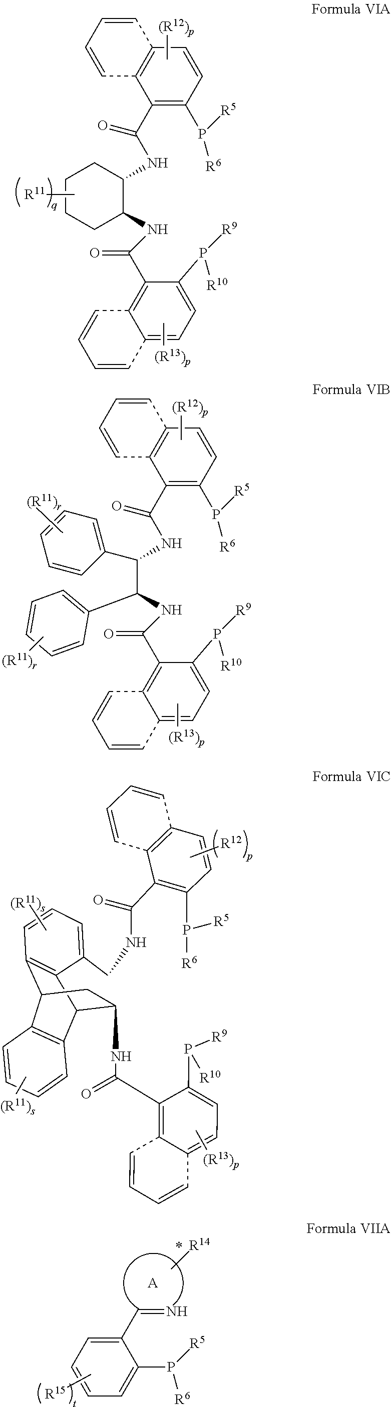
wherein:
each R¹¹ independently is selected from aliphatic, heteroaliphatic, haloaliphatic, aromatic, or an organic functional group;
each of R¹² and R¹³ independently, for each occurrence, is selected from aliphatic, heteroaliphatic, haloaliphatic, aromatic, or an organic functional group;
each R¹⁵ independently is selected from aliphatic, heteroaliphatic, haloaliphatic, aromatic, or an organic functional group;
p is an integer selected from 0 to 6;
q is an integer ranging from 0 to 10;
each r independently is an integer selected from 0 to 5;
each s independently is an integer selected from 0 to 4; and
t is an integer selected from 0 to 4.

19. A method, comprising using the chiral precatalyst according to claim 1 as a catalyst in a palladium-mediated enantioselective chemical reaction.

20. The method of claim 19, wherein the palladium-mediated enantioselective chemical reaction is an asymmetric allylation reaction.

21. The method of claim 20, wherein the asymmetric allylation reaction is an asymmetric malonation reaction, a desymmetrization reaction, or an asymmetric ring-opening allylation reaction.
